SEQUENCE LISTING (91 sequences)

SEQ ID NO: 1 - protein, 12 aa, Artificial Sequence
  Synthetic
GFSITTGGYWWT

SEQ ID NO: 2 - protein, 17 aa, Artificial Sequence
  Synthetic
GYIFSSGNTNYNPSIKS

SEQ ID NO: 3 - protein, 12 aa, Artificial Sequence
  Synthetic
CARAYGKLGFDY

SEQ ID NO: 4 - protein, 12 aa, Artificial Sequence
  Synthetic
TLSSQHSTYTIG

SEQ ID NO: 5 - protein, 11 aa, Artificial Sequence
  Synthetic
VNSDGSHSKGD

SEQ ID NO: 6 - protein, 9 aa, Artificial Sequence
  Synthetic
GSSDSSGYV

SEQ ID NO: 7 - protein, 5 aa, Artificial Sequence
  Synthetic
SDGIS

SEQ ID NO: 8 - protein, 16 aa, Artificial Sequence
  Synthetic
IISSGGNTYYASWAKG

SEQ ID NO: 9 - protein, 8 aa, Artificial Sequence
  Synthetic
VVGGTYSI

SEQ ID NO: 10 - protein, 13 aa, Artificial Sequence
  Synthetic
QSSQSVYNNDFLS

SEQ ID NO: 11 - protein, 7 aa, Artificial Sequence
  Synthetic
YASTLAS

SEQ ID NO: 12 - protein, 13 aa, Artificial Sequence
  Synthetic
TGTYGNSAWYEDA

SEQ ID NO: 13 - protein, 5 aa, Artificial Sequence
  Synthetic
SNAMI

SEQ ID NO: 14 - protein, 16 aa, Artificial Sequence
  Synthetic
AMDSNSRTYYATWAKG

SEQ ID NO: 15 - protein, 11 aa, Artificial Sequence
  Synthetic
GDGGSSDYTEM

SEQ ID NO: 16 - protein, 13 aa, Artificial Sequence
  Synthetic
QSSQSVYGNNELS

SEQ ID NO: 17 - protein, 7 aa, Artificial Sequence
  Synthetic
QASSLAS

SEQ ID NO: 18 - protein, 11 aa, Artificial Sequence
  Synthetic
LGEYSISADNH

SEQ ID NO: 19 - protein, 120 aa, Artificial Sequence
  Synthetic
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSS

SEQ ID NO: 20 - protein, 111 aa, Artificial Sequence
  Synthetic
QPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVL

SEQ ID NO: 21 - protein, 454 aa, Artificial Sequence
  Synthetic
METGLRWLLLVAVLKGVQCQSVEESGGRLVTPGTPLTLTCTVSGFSLSNNAINWVRQAPGKGLEWIGYIWSGGLTYYANWAEGRFTISKTSTTVDLKMTSPTIEDTATYFCARGINNSALWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 22 - protein, 239 aa, Artificial Sequence
  Synthetic
MDMRAPTQLLGLLLLWLPGARCADVVMTQTPASVSAAVGGTVTINCQASESISNYLSWYQQKPGQPPKLLIYRTSTLASGVSSRFKGSGSGTEYTLTISGVQCDDVATYYCQCTSGGKFISDGAAFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 23 - protein, 452 aa, Artificial Sequence
  Synthetic
METGLRWLLLVAVLKGVQCQSVEESGGRLVTPGTPLTLTCTVSGFTISDYDLSWVRQAPGEGLKYIGFIAIDGNPYYATWAKGRFTISKTSTTVDLKITAPTTEDTATYFCARGAGDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 24 - protein, 238 aa, Artificial Sequence
  Synthetic
MDTREPTQLLGLLLLWLPGARCADVVMTQTPASVSAAVGGTVTINCQSSKNVYNNNWLSWFQQKPGQPPKLLIYYASTLASGVPSRFRGSGSGTQFTLTISDVQCDDAATYYCAGDYSSSSDNGFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 25 - protein, 454 aa, Artificial Sequence
  Synthetic
METGLRWLLLVAVLKGVHCQSVEESGGRLVTPGTPLTLTCTASGFSRSSYDMSWVRQAPGKGLEWVGVISTAYNSHYASWAKGRFTISRTSTTVDLKMTSLTTEDTATYFCARGGSWLDLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 26 - protein, 239 aa, Artificial Sequence
  Synthetic
  misc_feature (3)..(3): Xaa can be any naturally occurring amino acid
MDXRAPTQLLGLLLLWLPGARCALVMTQTPASVSAAVGGTVTINCQSSQSVYDNDELSWYQQKPGQPPKLLIYALASKLASGVPSRFKGSGSGTQFALTISGVQCDDAATYYCQATHYSSDWYLTFGGGTEVVVKGFPVAPTVLLFPPSSDEVATGTVTIVCVANKYFPDVTVTWEVDGTTQTTGTENSKTPQNSADCTYNLSSTLTLTSTQYNSHKEYTCKVTQGTTSVVQSFSRKNC

SEQ ID NO: 27 - protein, 456 aa, Artificial Sequence
  Synthetic
METGLRWLLLVAVLKGVQCQSVEESGGRLVTPGTPLTLTCTVSGFSLSSYDMTWVRQAPGKGLEWIGIIYASGTTYYANWAKGRFTISKTSTTVDLKVTSPTIGDTATYFCAREGAGVSMTLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 28 - protein, 236 aa, Artificial Sequence
  Synthetic
MDMRAPTQLLGLLLLWLPGARCAYDMTQTPASVEVAVGGTVTIKCQASQSISTYLDWYQQKPGQPPKLLIYDASDLASGVPSRFKGSGSGTQFTLTISDLECADAATYYCQQGYTHSNVDNVFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 29 - protein, 459 aa, Artificial Sequence
  Synthetic
METGLRWLLLVAVLKGVQCQSVEESGGRLVSPGTPLTLTCTASGFSLSSYDMSWVRQAPGKGLEYIGIISSSGSTYYASWAKGRFTISKTSTTVDLEVTSLTTEDTATYFCSREHAGYSGDTGHLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVGIGPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 30 - protein, 236 aa, Artificial Sequence
  Synthetic
MDMRAPTQLLGLLLLWLPGARCAYDMTQTPASVEVAVGGTVAIKCQASQSVSSYLAWYQQKPGQPPKPLIYEASMLAAGVSSRFKGSGSGTDFTLTISDLECDDAATYYCQQGYSISDIDNAFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 31 - protein, 455 aa, Artificial Sequence
  Synthetic
METGLRWLLLVAVLKGVQCQSVEESGGRLVTPGTPLTLTCTVSGIDLSSDGISWVRQAPGKGLEWIGIISSGGNTYYASWAKGRFTISRTSTTVDLKMTSLTTEDTATYFCARVVGGTYSIWGQGTLVTVSSASTKGPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 32 - protein, 240 aa, Artificial Sequence
  Synthetic
MDTRAPTQLLGLLLLWLPGATFAQVLTQTASPVSAPVGGTVTINCQSSQSVYNNDFLSWYQQKPGQPPKLLIYYASTLASGVPSRFKGSGSGTQFTLTISDLECDDAATYYCTGTYGNSAWYEDAFGGGTEVVVKRTPVAPTVLLFPPSSAELATGTATIVCVANKYFPDGTVTWKVDGITQSSGINNSRTPQNSADCTYNLSSTLTLSSDEYNSHDEYTCQVAQDSGSPVVQSFSRKSC

SEQ ID NO: 33 - protein, 460 aa, Artificial Sequence
  Synthetic
METGLRWLLLVAVLKGVQCQSVEESGGRLVTPGTPLTLTCTVSGIDLSSNAMIWVRQAPREGLEWIGAMDSNSRTYYATWAKGRFTISRTSSITVDLKITSPTTEDTATYFCARGDGGSSDYTEMWGPGTLVTVSSASTKGPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 34 - protein, 239 aa, Artificial Sequence
  Synthetic
MDTRAPTQLLGLLLLWLPGATFAQAVVTQTTSPVSAPVGGTVTINCQSSQSVYGNNELSWYQQKPGQPPKLLIYQASSLASGVPSRFKGSGSGTQFTLTISDLECDDAATYYCLGEYSISADNHFGGGTEVVVKRTPVAPTVLLFPPSSAELATGTATIVCVANKYFPDGTVTWKVDGITQSSGINNSRTPQNSADCTYNLSSTLTLSSDEYNSHDEYTCQVAQDSGSPVVQSFSRKSC

SEQ ID NO: 35 - protein, 221 aa, Artificial Sequence
  Synthetic
QVQLVQSGGAVVQPGRSLRLSCAASGFTFSTYGMHWVRQAPGKGLEWVAAVSYDGSNKYYADFVKGRFTISRDNPKNTLYLQMNSLRADDTAVYYCARRGGLDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAA

SEQ ID NO: 36 - protein, 110 aa, Artificial Sequence
  Synthetic
LTQPPPASGTPGQQRVTISCSGSSSNIGSNTVNWYQQLPGTAPKLLIYYGNDQRPSGVPDRFSASKSGTSASLAISGLQSEDEAHYYCAAWDGSLNGGVIFGGGTKVTLG

SEQ ID NO: 37 - protein, 109 aa, Artificial Sequence
  Synthetic
VTQPPSASGTPGQRVTISCSGSSSNIGTNPVNWYQQLPGTAPKLLIYTTDQRPSGVPDRFSGSKSGTSASLAISGLQSEDEADYYCAAWDDSLSGLYVFGTGTKVTVLG

SEQ ID NO: 38 - protein, 109 aa, Artificial Sequence
  Synthetic
MTHTPLSLSVTPGQPASISCKSSQSLLHSDGKTYLYWYLQRPGQSPQPLIYEVSNRFSGVPDRFSGSGSGTDFTLKISRVQAEDVGVYYCMQSLQLWTFGQGTKVEIKR

SEQ ID NO: 39 - protein, 110 aa, Artificial Sequence
  Synthetic
MTQSPLSLPVTLGQPASISCRSSQSLIHSDGNTYLDWFQQRPGQSPRRLIYKVSNRDSGVPDRFSGSGSGTDFTLRISRVEAEDIGVYYCMQATHWPRTFGQGTKVEIKR

SEQ ID NO: 40 - protein, 110 aa, Artificial Sequence
  Synthetic
MTQSPLSLPVTLGQPASISCRSSQSLVDSAGNTFLHWFHQRPGQSPRRLIYKVSNRDSGVPDRFSGSGSGTDFTLKISRVEAEDVGVYYCMQGTHWPRTFGQGTKVEIKR

SEQ ID NO: 41 - protein, 110 aa, Artificial Sequence
  Synthetic
LTQSPLSLPVTLGQPASISCKSSQSLVDSDGNTYLNWFQQRPGQSPRRLIYKVSNRDSGVPDRFSGSGSGTDFTLKISRVEAEDVGVYYCMQGTHWPRTFGQGTKVEIKR

SEQ ID NO: 42 - protein, 110 aa, Artificial Sequence
  Synthetic
MTQSPLSLPVTLGQPASISCRSSQSLVHSDGNMYLNWFQQRPGQSPRRLIYKVSNRDSGVPDRFSGSGSGTDFTLKISRVEAEDVGVYYCMQATQPTWTFGQGTKLEIKR

SEQ ID NO: 43 - protein, 92 aa, Artificial Sequence
  Synthetic
MTQSPSSLSASVGDRVTITCQASQDISNYLNWYQQKPGKAPKLLIYDASNLETGVPSRFSGSGSGTDFTFTISSATYYCQQTYQGTKLEIKR

SEQ ID NO: 44 - protein, 105 aa, Artificial Sequence
  Synthetic
MTQSPSSLSASVGHPVTITCRASQSLISYLNWYHQKPGKAPKLLIYAASILQSGVPSRFSGSGSGTDFTLTISSLQPENFASYYCQHTDSFPRTFGHGTKVEIKR

SEQ ID NO: 45 - protein, 108 aa, Artificial Sequence
  Synthetic
LTQPPSASGTPGQGVTISCRGSTSNIGNNVVNWYQHVPGSAPKLLIWSNIQRPSGIPDRFSGSKSGTSASLAISGLQSEDQAVYYCAVWDDGLAGWVFGGGTTVTVLS

SEQ ID NO: 46 - protein, 105 aa, Artificial Sequence
  Synthetic
MTQAPVVSVALEQTVRITCQGDSLAIYYDFWYQHKPGQAPVLVIYGKNNRPSGIPHRFSGSSSNTDSLTITGAQAEDEADYYCNSRDSSGNHWVFGGGTNLTVLG

SEQ ID NO: 47 - protein, 111 aa, Artificial Sequence
  Synthetic
LTQSPLSLPVTLGQPASISCKSNQSLVHSDGNTYLNWFQQRPGQSPRRLIYKVSNRDSGVPDRFSGSGSGTDFTLKINRVEAEDVGVYYCMQGTQWPRTFGGQGTKLDIKR

SEQ ID NO: 48 - protein, 119 aa, Artificial Sequence
  Synthetic
QVQLQESGPGLVKPSETLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSS

SEQ ID NO: 49 - protein, 119 aa, Artificial Sequence
  Synthetic
QVQLQESGPGLVKPSQTLSLTCTVSGFSITTGGYWWTWIRQHPGKGLEWIGYIFSSGNTNYNPSIKSLVTISVDTSKNQFSLKLSSVTAADTAVYYCARAYGKLGFDYWGQGTLVTVSS

SEQ ID NO: 50 - protein, 119 aa, Artificial Sequence
  Synthetic
QVQLQESGPGLVKPSQTLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSS

SEQ ID NO: 51 - protein, 119 aa, Artificial Sequence
  Synthetic
QVQLQESGPGLVKPSETLSLTCTVSGFSITTGGYWWTWIRQPPGKGLEWIGYIFSSGNTNYNPSIKSRVTISRDTSKNQFSLKLSSVTAADTAVYYCARAYGKLGFDYWGQGTLVTVSS

SEQ ID NO: 52 - protein, 119 aa, Artificial Sequence
  Synthetic
QVQLQESGPGLVKPSETLSLTCTVSGFSITTGGYWWTWIRQPPGKGLEWIGYIFSSGNTNYNPSIKSRVTISVDTSKNQFSLKLSSVTAADTARYYCARAYGKLGFDYWGQGTLVTVSS

SEQ ID NO: 53 - protein, 120 aa, Artificial Sequence
  Synthetic
QVQLQESGPGLVKPSETLSLTCTVSGFSITTGGYWWTWIRQPPGKGLEWIGYIFSSGNTNYNPSIKSRISITRDTSKNQFFLQLNSVTTEGDTARYYCARAYGKLGFDYWGQGTLVTVSS

SEQ ID NO: 54 - protein, 111 aa, Artificial Sequence
  Synthetic
QLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 55 - protein, 111 aa, Artificial Sequence
  Synthetic
LPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 56 - protein, 15 aa, Artificial Sequence
  Synthetic
GGGGSGGGGSGGGGS

SEQ ID NO: 57 - protein, 247 aa, Artificial Sequence
  Synthetic
QPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVLRAAASSGGGGSGGGGSGGGGSQPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVLRAAA

SEQ ID NO: 58 - protein, 253 aa, Artificial Sequence
  Synthetic
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSSGGGGSGGGGSGGGGSQVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTV

SEQ ID NO: 59 - protein, 245 aa, Artificial Sequence
  Synthetic
QVQLQESGPGLVKPSETLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 60 - protein, 245 aa, Artificial Sequence
  Synthetic
QVQLQESGPGLVKPSQTLSLTCTVSGFSITTGGYWWTWIRQHPGKGLEWIGYIFSSGNTNYNPSIKSLVTISVDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 61 - protein, 245 aa, Artificial Sequence
  Synthetic
QVQLQESGPGLVKPSQTLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 62 - protein, 245 aa, Artificial Sequence
  Synthetic
QVQLQESGPGLVKPSETLSLTCTVSGFSITTGGYWWTWIRQPPGKGLEWIGYIFSSGNTNYNPSIKSRVTISRDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 63 - protein, 245 aa, Artificial Sequence
  Synthetic
QVQLQESGPGLVKPSETLSLTCTVSGFSITTGGYWWTWIRQPPGKGLEWIGYIFSSGNTNYNPSIKSRVTISVDTSKNQFSLKLSSVTAADTARYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 64 - protein, 246 aa, Artificial Sequence
  Synthetic
QVQLQESGPGLVKPSETLSLTCTVSGFSITTGGYWWTWIRQPPGKGLEWIGYIFSSGNTNYNPSIKSRISITRDTSKNQFFLQLNSVTTEGDTARYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 65 - protein, 245 aa, Artificial Sequence
  Synthetic
QVQLQESGPGLVKPSETLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 66 - protein, 245 aa, Artificial Sequence
  Synthetic
QVQLQESGPGLVKPSQTLSLTCTVSGFSITTGGYWWTWIRQHPGKGLEWIGYIFSSGNTNYNPSIKSLVTISVDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 67 - protein, 245 aa, Artificial Sequence
  Synthetic
QVQLQESGPGLVKPSQTLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 68 - protein, 245 aa, Artificial Sequence
  Synthetic
QVQLQESGPGLVKPSETLSLTCTVSGFSITTGGYWWTWIRQPPGKGLEWIGYIFSSGNTNYNPSIKSRVTISRDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 69 - protein, 245 aa, Artificial Sequence
  Synthetic
QVQLQESGPGLVKPSETLSLTCTVSGFSITTGGYWWTWIRQPPGKGLEWIGYIFSSGNTNYNPSIKSRVTISVDTSKNQFSLKLSSVTAADTARYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 70 - protein, 246 aa, Artificial Sequence
  Synthetic
QVQLQESGPGLVKPSETLSLTCTVSGFSITTGGYWWTWIRQPPGKGLEWIGYIFSSGNTNYNPSIKSRISITRDTSKNQFFLQLNSVTTEGDTARYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 71 - protein, 246 aa, Artificial Sequence
  Synthetic
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSSGGGGSGGGGSGGGGSQPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVL

SEQ ID NO: 72 - protein, 5 aa, Artificial Sequence
  Synthetic
SYWMN

SEQ ID NO: 73 - protein, 17 aa, Artificial Sequence
  Synthetic
QIWPGDGDTNYNGKFKG

SEQ ID NO: 74 - protein, 15 aa, Artificial Sequence
  Synthetic
RETTTVGRYYYAMDY

SEQ ID NO: 75 - protein, 15 aa, Artificial Sequence
  Synthetic
KASQSVDYDGDSYLN

SEQ ID NO: 76 - protein, 7 aa, Artificial Sequence
  Synthetic
DASNLVS

SEQ ID NO: 77 - protein, 9 aa, Artificial Sequence
  Synthetic
QQSTEDPWT

SEQ ID NO: 78 - protein, 124 aa, Artificial Sequence
  Synthetic
QVQLQESGAELVRPGSSVKISCKASGYAFSSYWMNWVKQRPGQGLEWIGQIWPGDGDTNYNGKFKGKATLTADESSSTAYMQLSSLRSEDSAVYSCARRETTTVGRYYYAMDYWGQGTTVTVSS

SEQ ID NO: 79 - protein, 112 aa, Artificial Sequence
  Synthetic
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKR

SEQ ID NO: 80 - protein, 223 aa, Artificial Sequence
  Synthetic
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 81 - protein, 220 aa, Artificial Sequence
  Synthetic
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIKRTVAAPSVFIFPPSDEQLKSGTASVVCLLNNFYPREAKVQWKVDNALQSGNSQESVTEQDSGGGGSNYHLENEVARLKKLGGGGSDSTYSLSSTLTLSKADYEKHKVYACEVT

SEQ ID NO: 82 - protein, 223 aa, Artificial Sequence; identical to SEQ ID NO: 80
  Synthetic

SEQ ID NO: 83 - protein, 124 aa, Artificial Sequence
  Synthetic
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 84 - protein, 111 aa, Artificial Sequence
  Synthetic
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 85 - protein, 464 aa, Artificial Sequence
  Synthetic
MEWSWIFLFLLSGTAGVHSEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 86 - protein, 121 aa, Artificial Sequence
  Synthetic
EVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSS

SEQ ID NO: 87 - protein, 239 aa, Artificial Sequence
  Synthetic
MRCLAEFLGLLVLWIPGAIGDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 88 - protein, 115 aa, Artificial Sequence
  Synthetic
DIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRAD

SEQ ID NO: 89 - protein, 116 aa, Artificial Sequence
  Synthetic
DIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRSDP

SEQ ID NO: 90 - protein, 473 aa, Artificial Sequence
  Synthetic
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSSGGGGSGGGGSGGGGSQPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVLRAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 21 aa, Artificial Sequence
  Synthetic
MALPVTALLLPLALLLHAARP

(19) United States
(12) Patent Application Publication
(10) Pub. No.: US 2023/0321239 A1
(43) Pub. Date: Oct. 12, 2023

(54) CHIMERIC ANTIGEN RECEPTOR T CELLS FOR TREATING AUTOIMMUNITY

(71) Applicants: H. LEE MOFFITT CANCER CENTER AND RESEARCH INSTITUTE INC., Tampa, FL (US); REGENTS OF THE UNIVERSITY OF MINNESOTA, Minneapolis, MN (US)

(72) Inventors: Brian BETTS, Minneapolis, MN (US); Marco DAVILA, Tampa, FL (US)

(73) Assignees: H. LEE MOFFITT CANCER CENTER AND RESEARCH INSTITUTE INC., Tampa, FL (US); REGENTS OF THE UNIVERSITY OF MINNESOTA, Minneapolis, MN (US)

(21) Appl. No.: 18/041,542

(22) PCT Filed: Aug. 13, 2021
(86) PCT No.: PCT/US2021/045950

§ 371 Date: Feb. 13, 2023

Related U.S. Application Data
(60) Provisional application No. 63/072,093, filed on Aug. 28, 2020.
(60) Provisional application No. 63/065,841, filed on Aug. 14, 2020.

(51) Int. Cl. A61K 39/00; C07K 16/28; C07K 14/725; A61P 37/06; C12N 5/0783; C07K 14/705

(52) U.S. Cl. CPC A61K 39/4631; A61K 39/4611; A61K 39/4633; A61P 37/06; C07K 14/70503; C07K 14/70507; C07K 14/7051; C07K 14/70521; C07K 14/70532; C07K 14/70578; C07K 16/2803; C12N 5/0636; C07K 2317/622

(57) ABSTRACT

Disclosed are compositions and methods for treating autoimmune diseases such as lupus, including immune cells expressing at least a chimeric antigen receptor (CAR) polypeptides that binds CD83 and uses thereof for suppressing and/or killing autoreactive cells in a subject having an autoimmune disease.

### CROSS REFERENCE TO RELATED APPLICATIONS

This application claims the benefit of the filing dates of U.S. Provisional Application No. 63/072,093, filed Aug. 28, 2020 and U.S. Provisional Application No. 63/065,841, filed Aug. 14, 2020, the entire contents of each of which are incorporated by reference herein.

### BACKGROUND

Many people suffer from autoimmune diseases. For example, systemic lupus erythematosus (SLE) is a heterogeneous autoimmune disease that afflicts many people and is capable of catastrophic organ damage. Mortality among SLE patients is over 5 times greater than age-matched controls (Singh RR, et al. Lupus. 2018 27(10):1577-81). Glucocorticoids are often used in treating SLE, but their efficacy is limited, and such broad immunosuppression increases the risk for opportunistic infections (Fanouriakis A, et al. Annals of the Rheumatic Diseases. 2019 78(6):736-45). Long-term exposure to glucocorticoids is also associated with diabetes, osteoporosis, and avascular necrosis. Attempts to test biologic therapeutics in SLE have largely failed, leaving few alternatives to respond to steroid-refractory disease (Fanouriakis A, et al. Annals of the Rheumatic Diseases. 2019 78(6):736-45).

### SUMMARY

Disclosed herein are compositions and methods of suppressing autoreactive lymphocytes, such as B lymphocytes, in a subject in need thereof, e.g. a subject having an autoimmune disease. Such compositions and methods may involve immune cells genetically modified to express a chimeric antigen receptor (CAR) targeting CD83, and optionally an additional CAR targeting CD19. As disclosed herein, CD83 is differentially expressed on autoreactive lymphocytes such as B cells and T cells. Thus, immune cells expressing an anti-CD83 CAR can target autoreactive lymphocytes, thereby benefiting treatment of autoimmune diseases such as lupus.

Therefore, some aspects of the present disclosure features a method of inhibiting autoreactive lymphocytes in a subject, the method comprising administering to the subject an effective amount of immune cells genetically modified to express an chimeric antigen receptor (CAR) polypeptide comprising a CD83 antigen binding domain, a transmembrane domain, an intracellular signaling domain, and a co-stimulatory signaling region. Such immune cells would be effective in inhibiting autoreactive lymphocytes in the subject.

In some embodiments, the CAR polypeptide targeting CD83 (e.g., human CD83) may be defined by the formula: SP-CD83-HG-TM-CSR-ISD; or SP-CD83-HG-TM-ISD-CSR. “SP” represents a signal peptide. “CD83” represents a CD83 antigen binding region. “HG” represents and optional hinge domain. “TM” represents a transmembrane domain. “CSR” represents a co-stimulatory signaling region. “ISD” represents an intracellular signaling domain. “-” represents an optional bivalent linker. In some instances, two adjacent elements in any of the formulas may be connected with the bivalent linker. In other instances, two adjacent elements in the formula may be connected directly without the bivalent linker.

In other aspects, the present disclosure provides a method of selectively inhibiting autoreactive lymphocytes in a subject, the method comprising administering to the subject an effective amount of immune cells genetically modified to express a first chimeric antigen receptor (CAR) polypeptide comprising a CD83 antigen binding domain and a second CAR polypeptide comprising a CD19 antigen binding domain, thereby selectively inhibiting autoreactive B lymphocytes that express both CD83 and CD19 in the subject.

In some embodiments, the dual CAR T cells disclosed herein can have incomplete endodomains and only function when both CARs bind their target (e.g. CD83 and CD19). For example, one CAR can include only the CD3ζ domain, and the other CAR can include only the co-stimulatory domain(s). In these embodiments, dual CAR T cell activation requires co-expression and activation by binding to both targets.

In some embodiments, the first CAR polypeptide that targets CD83 is defined by the formula SP-CD83-HG-TM-SD, and the second CAR that targets CD19 is defined by the formula SP-CD19-HG-TM-CSR. Alternatively, the first CAR polypeptide that targets CD83 is defined by the formula SP-CD83-HG-TM-CSR, and the second CAR that targets CD19 is defined by the formula SP-CD19-HG-TM-SD. In these formulas, “SP” represents an optional signal peptide; “CD83” represents a CD83 antigen binding region; “CD19” represents a CD19 antigen binding region; “HG” represents an optional hinge domain; “TM” represents a transmembrane domain; “CSR” represents one or more co-stimulatory signaling regions; “SD” represents a signaling domain, and “-” represents an optional peptide bond or linker.

In any of the methods disclosed herein, the subject can be a human patient having an autoimmune disease. In some examples, the autoimmune disease is a B cell-mediated autoimmune disease, such as autoimmune diseases where autoreactive B cells are shown to express CD83. In some examples, the subject has systemic lupus erythematosus (SLE). The method disclosed herein may treat or prevent one or more symptoms of said SLE in the subject In some embodiments, the disclosed CAR systems and methods can be used to treat B cell-mediated autoimmune diseases,

The CAR systems and methods can be used to treat any appropriate autoimmune condition involving autoreactive lymphocytes expressing CD83. Examples include, but are not limited to, Achalasia, Acute disseminated encephalomyelitis (ADEM), Addison’s disease, Adiposis dolorosa, Adult Still’s disease, Agammaglobulinemia, Alopecia areata, Amyloidosis, Ankylosing spondylitis, Anti-GBM/Anti-TBM nephritis, Anti-N-Methyl-D-Aspartate (Anti-NMDA) receptor encephalitis, Antiphospholipid syndrome, Antisynthetase syndrome, Aplastic Anemia, Autoimmune angioedema, Autoimmune dysautonomia, Autoimmune encephalomyelitis, Autoimmune enteropathy, Autoimmune hepatitis, Autoimmune inner ear disease (AIED), Autoimmune lymphoproliferative syndrome, Autoimmune myocarditis, Autoimmune oophoritis, Autoimmune orchitis, Autoimmune pancreatitis, Autoimmune polyendocrine syndrome (APS) type 1, Autoimmune polyendocrine syndrome (APS) type 2, Autoimmune polyendocrine syndrome (APS) type 3, Autoimmune retinopathy, Autoimmune urticaria, Axonal & neuronal neuropathy (AMAN), Baló disease, Behcet’s disease, Benign mucosal pemphigoid, Bickerstaff’s encephalitis, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan’s syndrome, Cold agglutinin disease, Congenital heart block, Coxsackie myocarditis, CREST syndrome, Crohn’s disease, Dermatitis herpetiformis, Dermatomyositis, Devic’s disease (neuromyelitis optica), Discoid lupus, Dressler’s syndrome, Drug-induced lupus, Endometriosis, Enthesitis-related arthritis, Eosinophilic esophagitis (EoE), Eosinophilic fasciitis, Epidermolysis bullosa acquisita, Erythema nodosum, Essential mixed cryoglobulinemia, Evans syndrome, Felty Syndrome, Fibromyalgia, Fibrosing alveolitis, Giant cell arteritis (temporal arteritis), Giant cell myocarditis, Glomerulonephritis, Goodpasture’s syndrome, Granulomatosis with Polyangiitis, Graves’ disease, Guillain-Barre syndrome, Hasimoto’s encephalopathy, Hashimoto’s thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgA Vasculitis, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lupus Nephritis, Lupus Vasculitis, Lyme disease chronic, Meniere’s disease, Microscopic polyangiitis (MPA), Mixed connective tissue disease (MCTD), Mooren’s ulcer, Morphea, Mucha-Habermann disease,Multifocal Motor Neuropathy (MMN) or MMNCB, Multiple sclerosis, Myasthenia gravis, Myositis, Narcolepsy, Neonatal Lupus, Neuromyelitis optica, Neuromyotonia, Neutropenia, Ocular cicatricial pemphigoid, Optic neuritis, Ord’s thyroiditis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), Pityriasis lichenoides et varioliforis acuta, POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cholangitis, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud’s phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Schnitzler syndrome, Scleritis, Scleroderma, Sjögren’s syndrome, Sperm & testicular autoimmunity, Stiff person syndrome (SPS), Subacute bacterial endocarditis (SBE), Susac’s syndrome, Sydenham’s chorea, Sympathetic ophthalmia (SO), Systemic lupus erythematosus (SLE), Takayasu’s arteritis, Temporal arteritis/Giant cell arteritis, Thrombocytopenia, Thrombocytopenic purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Urticarial vasculitis, Uveitis, Vasculitis, Vitiligo, and Vogt-Koyanagi-Harada Disease.

Also disclosed herein are dual-CAR systems to selectively eliminate only autoreactive B cells that express both CD83 and CD19, thereby avoiding off-target effects on CD83+ non-B cell bystander cells or complete B cell aplasia as observed with CD19 CAR T cells alone. The objective is to eliminate the need for broad immunosuppression, like steroids, in treating SLE by using CD83-targeted cell therapy. Such a dual-CAR system may involve a population of immune cells genetically engineered to express both a CAR polypeptide targeting CD83 and a CAR polypeptide targeting CD19 as disclosed herein.

The immune cells disclosed herein, which can be used in any of the methods disclosed herein for suppressing autoreactive lymphocytes, may comprise alpha-beta T cells, gamma-delta T cells, Natural Killer (NK) cells, Natural Killer T (NKT) cells, B cells, innate lymphoid cells (ILCs), cytokine induced killer (CIK) cells, cytotoxic T lymphocytes (CTLs), lymphokine activated killer (LAK) cells, regulatory T cells (Tregs), or a combination thereof. In some embodiments, the immune cells comprise T cells such as CTLs.

In some embodiments, Tregs are able to kill autoreactive or alloreactive B cells expressing CD83, while only suppressing CD83-expressing T cells. In some embodiments, the disclosed method involves treating B-cell mediated autoimmunity, such as SLE, with Treg cells expressing an anti-CD83 CAR or both an anti-CD83 CAR and an anti-CD19 CAR as disclosed herein.

In some embodiments, the anti-CD83 CAR polypeptide or the anti-CD19 CAR polypeptide may comprise an antibody fragment that specifically binds CD83 or CD19, respectively. For example, the antigen-binding domain can be a Fab or a single-chain variable fragment (scFv) of an antibody that specifically binds CD83 or CD19. In some embodiments, the anti-CD83 CAR, the anti-CD19 CAR, or both may comprise a natural ligand of CD83 or CD19, or a variant and/or fragment thereof capable of binding CD83 or CD19.

Any of the anti-CD83 CAR polypeptide may comprise a CD83 antigen binding domain, which optionally can be a single-chain variable fragment (scFv) of an antibody that specifically binds CD83. In some embodiments, the anti-CD83 scFv comprises a variable heavy (V_{H}) domain having heavy chain (HC) CDR1, CDR2 and CDR3 regions; and a variable light (V_{L}) domain having light chain (LC) CDR1, CDR2 and CDR3 regions. The HC CDR1 may comprise the amino acid sequence SEQ ID NO:1, SEQ ID NO:7, or SEQ ID NO:13; the HC CDR2 comprises the amino acid sequence SEQ ID NO:2, SEQ ID NO:8, or SEQ ID NO:14; the HC CDR3 comprises the amino acid sequence SEQ ID NO:3, SEQ ID NO:9, or SEQ ID NO:15; the LC CDR1 comprises the amino acid sequence SEQ ID NO:4, SEQ ID NO:10, or SEQ ID NO:16; the LC CDR2 comprises the amino acid sequence SEQ ID NO:5, SEQ ID NO:11, or SEQ ID NO:17; and/or the LC CDR3 comprises the amino acid sequence SEQ ID NO:6, SEQ ID NO:12, or SEQ ID NO:18.

In some examples, the anti-CD83 scFv comprises:a HC CDR1 comprising the amino acid sequence of SEQ ID NO: 1; a HC CDR2 comprising the amino acid sequence of SEQ ID NO: 2; a HC CDR2 comprising the amino acid sequence of SEQ ID NO: 3; a LC CDR1 comprising the amino acid sequence of SEQ ID NO: 4; a LC CDR2 comprising the amino acid sequence of SEQ ID NO: 5; and a LC CDR3 comprising the amino acid sequence of SEQ ID NO: 6.

In some embodiments, the anti-CD83 scFv V_{H} domain comprises the amino acid sequence of SEQ ID NO:19, SEQ ID NO:48, SEQ ID NO:49, SEQ ID NO:50, SEQ ID NO:51, SEQ ID NO:52, or SEQ ID NO:53; and/or wherein the anti-CD83 scFv V_{L} domain comprises the amino acid sequence of SEQ ID NO:20, SEQ ID NO:54, or SEQ ID NO:55. In some examples, the anti-CD83 scFv comprises a V_{H} comprising the amino acid sequence of SEQ ID NO:19, and a V_{L} comprising the amino acid sequence of SEQ ID NO:20.

In some examples, the anti-CD83 scFv comprises the amino acid sequence of SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, or SEQ ID NO:71. In one specific example, the anti-CD83 scFv comprises the amino acid sequence of SEQ ID NO: 71.

As with other CARs, the anti-CD83 CAR polypeptides, the anti-CD19 CAR polypeptides, or both can also contain a transmembrane domain and an endodomain capable of activating the immune cells disclosed herein. For example, the endodomain can contain an intracellular signaling domain, one or more co-stimulatory signaling regions, or a combination thereof.

In some embodiments, the intracellular signaling domain can be a CD3 zeta (CD3ζ) signaling domain. In some embodiments, the costimulatory signaling region comprises the cytoplasmic domain of CD28, 4-1BB, or a combination thereof. In some cases, the costimulatory signaling region may contain 1, 2, 3, or 4 cytoplasmic domains of one or more intracellular signaling and/or costimulatory molecules. In some embodiments, the co-stimulatory signaling region contains one or more mutations in the cytoplasmic domains of CD28 and/or 4-1BB that enhance signaling.

In some specific examples, the anti-CD83 CAR polypeptide disclosed herein may comprise the amino acid sequence of SEQ ID NO: 90.

In some embodiments, the anti-CD83 CAR polypeptides and/or the anti-CD19 CAR polypeptides contains an incomplete endodomain. For example, the CAR polypeptide can contain only an intracellular signaling domain or a co-stimulatory domain, but not both. In these embodiments, the immune cells expressing such a CAR may not be activated unless they also express a second CAR polypeptide (or an endogenous T-cell receptor) that contains the missing domain for binding their respective antigens.

Therefore, in some embodiments, the genetically engineered immune cells may express an anti-CD83 CAR polypeptide, which contains a CD3 zeta (CD3ζ) signaling domain but does not contain a costimulatory signaling region (CSR) and an anti-CD19 CAR polypeptide, which contains a cytoplasmic domain of CD28, 4-1BB, or a combination thereof, but does not contain a CD3 zeta (CD3ζ) signaling domain (SD). Alternatively, the genetically engineered immune cells may express an anti-CD19 CAR polypeptide, which contains a CD3 zeta (CD3C) signaling domain but does not contain a costimulatory signaling region (CSR) and an anti-CD84 CAR polypeptide, which contains a cytoplasmic domain of CD28, 4-1BB, or a combination thereof, but does not contain a CD3 zeta (CD3ζ) signaling domain (SD).

Also disclosed are isolated nucleic acids comprising sequences encoding any of the CAR polypeptides or any of the dual CAR combinations disclosed herein, vectors comprising these isolated nucleic acids, and immune cells containing these vectors.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B show autoreactive SLE B cells over express CD83. PBMCs from a patient with SLE (black histogram) versus a healthy donor (gray histogram) stimulated with CD3/28 beads (cell:bead ratio 1:30) for 6 hours (FMO unfilled histogram). CD19+ B cells (FIG. 1A) and CD4+ T cells (FIG. 1B) were stained for CD83 expression at baseline and after stimulation. Geometric MFI is shown for each test histogram. A representative experiment is shown.

FIGS. 2A to 2E show human CD83-targeted CAR T construct and functional characteristics. FIG. 2A is a bar graph showing the amount (mean ± SEM) of T cells expressing the eGFP reporter post production. FIGS. 2B and 2C show the amount of IFNγ and IL-2 released by mock transduced or CD83 CAR T cells after stimulation with CD83+ DCs. FIG. 2D shows cytotoxicity of CD83 CAR T cells or mock transduced T cells co-cultured with CD83+ DCs. 1 representative experiment of 2 is shown. NSG mice received 25×10⁶ human PBMCs and were inoculated with low (1×10⁶) or high dose (10×10⁶) CD83 CAR or (1-10×10⁶) mock transduced T cells. FIG. 2E is a graph showing survival. **P=0.001-0.01, ***P=0.0001-0.001, ****P<0.0001.

FIG. 3 shows an embodiment of CD19/CD83 CAR T cells in SLE. Scenario 1: autoreactive B cells mediate SLE pathology, leading to renal, cutaneous, and musculoskeletal tissue damage. Host antiviral activity remains unperturbed. Scenario 2: anti-CD83 CAR-T cells kill autoreactive CD83+ CD19+ B cells, as well as mature CD83+ dendritic cells. Targeting dendritic cells could impair host immunity. Scenario 3: anti-CD19 CAR-T cells kill All B cells, leading to B cell aplasia. Targeting all B cells could cause hypogammaglobulinemia and impair host immunity. Scenario 4: bispecific anti-CD19/anti-CD83 CAR-T cells only kill targets co-expressing CD19 and CD83. Autoreactive B cells are killed, but antiviral B cells and dendritic cells are spared.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in °C, and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Definitions

The term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class from any species, including any of the human classes: IgG, IgM, IgA, IgD, and IgE. In exemplary embodiments, antibodies used with the methods and compositions described herein are derivatives of the IgG class. In addition to intact immunoglobulin molecules, also included in the term “antibodies” are fragments or polymers of those immunoglobulin molecules, and human or humanized versions of immunoglobulin molecules that selectively bind the target antigen.

The term “antibody fragment” refers to any derivative of an antibody which is less than full-length. In exemplary embodiments, the antibody fragment retains at least a significant portion of the full-length antibody’s specific binding ability. Examples of antibody fragments include, but are not limited to, Fab, Fab′, F(ab′)2, scFv, Fv, dsFv diabody, Fc, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

The term “antigen binding site” refers to a region of an antibody that specifically binds an epitope on an antigen.

The term “aptamer” refers to oligonucleic acid or peptide molecules that bind to a specific target molecule. These molecules are generally selected from a random sequence pool. The selected aptamers are capable of adapting unique tertiary structures and recognizing target molecules with high affinity and specificity. A “nucleic acid aptamer” is a DNA or RNA oligonucleic acid that binds to a target molecule via its conformation, and thereby inhibits or suppresses functions of such molecule. A nucleic acid aptamer may be constituted by DNA, RNA, or a combination thereof. A “peptide aptamer” is a combinatorial protein molecule with a variable peptide sequence inserted within a constant scaffold protein. Identification of peptide aptamers is typically performed under stringent yeast dihybrid conditions, which enhances the probability for the selected peptide aptamers to be stably expressed and correctly folded in an intracellular context.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “chimeric antigen receptor (CAR)” refers to an artificial immune cell receptor that is engineered to recognize and bind to an antigen expressed by undesired cells, for example, disease cells such as autoreactive lymphocytes. A T cell that expresses a CAR polypeptide is referred to as a CAR T cell.

The term “engineered antibody” refers to a recombinant molecule that comprises at least an antibody fragment comprising an antigen binding site derived from the variable domain of the heavy chain and/or light chain of an antibody and may optionally comprise the entire or part of the variable and/or constant domains of an antibody from any of the Ig classes (for example IgA, IgD, IgE, IgG, IgM and IgY).

The term “epitope” refers to the region of an antigen to which an antibody binds preferentially and specifically. A monoclonal antibody binds preferentially to a single specific epitope of a molecule that can be molecularly defined. In the present invention, multiple epitopes can be recognized by a multispecific antibody.

The term “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

The term “Fab fragment” refers to a fragment of an antibody comprising an antigen-binding site generated by cleavage of the antibody with the enzyme papain, which cuts at the hinge region N-terminally to the inter-H-chain disulfide bond and generates two Fab fragments from one antibody molecule.

The term “F(ab′)2 fragment” refers to a fragment of an antibody containing two antigen-binding sites, generated by cleavage of the antibody molecule with the enzyme pepsin which cuts at the hinge region C-terminally to the inter-H-chain disulfide bond.

The term “Fc fragment” refers to the fragment of an antibody comprising the constant domain of its heavy chain.

The term “Fv fragment” refers to the fragment of an antibody comprising the variable domains of its heavy chain and light chain.

“Gene construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc), may be transfected into cells, e.g. in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The term “identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance. In some embodiments, the linker may be a peptide linker.

The term “multivalent antibody” refers to an antibody or engineered antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody’s antigen recognition sites all bind the same epitope. A “bispecific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “peptide,” “protein,” and “polypeptide” are used interchangeably to refer to a natural or synthetic molecule comprising two or more amino acids linked by the carboxyl group of one amino acid to the alpha amino group of another.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The terms “polypeptide fragment” or “fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

The term “protein domain” refers to a portion of a protein, portions of a protein, or an entire protein showing structural integrity; this determination may be based on amino acid composition of a portion of a protein, portions of a protein, or the entire protein.

The term “single chain variable fragment or scFv” refers to an Fv fragment in which the heavy chain domain and the light chain domain are linked. One or more scFv fragments may be linked to other antibody fragments (such as the constant domain of a heavy chain or a light chain) to form antibody constructs having one or more antigen recognition sites.

A “spacer” as used herein refers to a peptide that joins the proteins comprising a fusion protein. Generally a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non- target cell or tissue. Nevertheless, specific delivery, may be distinguished as mediated through specific recognition of the target molecule. Typically specific delivery results in a much stronger association between the delivered molecule and cells bearing the target molecule than between the delivered molecule and cells lacking the target molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The terms “transformation” and “transfection” mean the introduction of a nucleic acid, e.g., an expression vector, into a recipient cell including introduction of a nucleic acid to the chromosomal DNA of said cell.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “variant” refers to an amino acid or peptide sequence having conservative amino acid substitutions, non-conservative amino acid subsitutions (i.e. a degenerate variant), substitutions within the wobble position of each codon (i.e. DNA and RNA) encoding an amino acid, amino acids added to the C-terminus of a peptide, or a peptide having 60%, 70%, 80%, 90%, 95%, 96%, 97%, 98%, 99% sequence identity to a reference sequence.

The term “vector” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element).

Disclosed herein are chimeric antigen receptors (CAR) that target CD83 on antigen-presenting cells. Also disclosed are immune cells that are engineered to express these CARs. CAR T cells expressing these CARs can suppress and/or kill alloreactive and/or autoreactive cells, such as B cells. Therefore, also disclosed are methods for treating or preventing autoimmunity in a subject that involves adoptive transfer of the disclosed immune cells engineered to express the disclosed CD83-specific CARs. Also disclosed are methods for treating or preventing autoimmunity in a subject that involves adoptive transfer of the disclosed dual CAR cells engineered to express CD83-specific and CD19-specific CARs wherein the dual CAR cells suppress and/or kill the target cell only when both CARs bind their target antigens.

### Chimeric Antigen Receptors (CAR)

CARs generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3:35-45). Disclosed herein is a CD83-specific chimeric antigen receptor (CAR) that can be that can be expressed in immune cells to suppress alloreactive and/or autoreactive donor cells. In some embodiments, the CARs disclosed herein may comprise a CD83 binding moiety. In some instances, the CD83 binding moiety may be an aptamer capable of binding to CD83.

### Anti-CD83 CAR Polypeptides

In some embodiments, a CAR polypeptide may be generally made up of three domains: an ectodomain, a transmembrane domain, and an endodomain. The ectodomain comprises the CD83-binding region and is responsible for antigen recognition. It also optionally contains a signal peptide (SP) so that the CAR can be glycosylated and anchored in the cell membrane of the immune cell. The transmembrane domain (TD), is as its name suggests, connects the ectodomain to the endodomain and resides within the cell membrane when expressed by a cell. The endodomain is the business end of the CAR that transmits an activation signal to the immune cell after antigen recognition. For example, the endodomain can contain an intracellular signaling domain (ISD) and optionally a co-stimulatory signaling region (CSR).

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41BB, and ICOS, that are able to enhance T-cell activation by T-cell receptors.

In some embodiments, the disclosed CAR is defined by the formula:

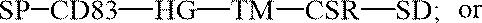

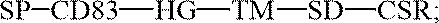

wherein “SP” represents an optional signal peptide,
wherein “CD83” represents a CD83-binding region,
wherein “HG” represents an optional hinge domain,
wherein “TM” represents a transmembrane domain,
wherein “CSR” represents one or more co-stimulatory signaling regions,
wherein “SD” represents a signaling domain, and
wherein “-” represents a peptide bond or linker.

Additional CAR constructs are described, for example, in Fresnak AD, et al. Engineered T cells: the promise and challenges of cancer immunotherapy. Nat Rev Cancer. 2016 Aug 23;16(9):566-81, which is incorporated by reference in its entirety for the teaching of these CAR models.

In some embodiments, the anti-CD83 scFv can comprise a variable heavy (V_{H}) domain having CDR1, CDR2 and CDR3 sequences and a variable light (V_{L}) domain having CDR1, CDR2 and CDR3 sequences. For example, in some embodiments, the CDR1 sequence of the V_{H} domain comprises the amino acid sequence GFSITTGGYWWT (SEQ ID NO:1), SDGIS (SEQ ID NO:7), or SNAMI (SEQ ID NO:13); CDR2 sequence of the V_{H} domain comprises the amino acid sequence GYIFSSGNTNYNPSIKS (SEQ ID NO:2), IISSGGNTYYASWAKG (SEQ ID NO:8), or AMDSNSRTYYATWAKG (SEQ ID NO:14); CDR3 sequence of the V_{H} domain comprises the amino acid sequence CARAYGKLGFDY (SEQ ID NO:3), VVGGTYSI (SEQ ID NO:9), or GDGGSSDYTEM (SEQ ID NO:15); CDR1 sequence of the V_{L} comprises the amino acid sequence TLSSQHSTYTIG (SEQ ID NO:4), QSSQSVYNNDFLS (SEQ ID NO:10), or QSSQSVYGNNELS (SEQ ID NO:16); CDR2 sequence of the V_{L} domain comprises the amino acid sequence VNSDGSHSKGD (SEQ ID NO:5), YASTLAS (SEQ ID NO:11), or QASSLAS (SEQ ID NO:17); and CDR3 sequence of the V_{L} domain comprises the amino acid sequence GSSDSSGYV (SEQ ID NO:6), TGTYGNSAWYEDA (SEQ ID NO:12), or LGEYSISADNH (SEQ ID NO:18).

For example, in some embodiments, the CDR1 sequence of the V_{H} domain comprises the amino acid sequence GFSITTGGYWWT (SEQ ID NO:1), CDR2 sequence of the V_{H} domain comprises the amino acid sequence GYIFSSGNTNYNPSIKS (SEQ ID NO:2), CDR3 sequence of the V_{H} domain comprises the amino acid sequence CARAYGKLGFDY (SEQ ID NO:3), CDR1 sequence of the V_{L} comprises the amino acid sequence TLSSQHSTYTIG (SEQ ID NO:4), CDR2 sequence of the V_{L} domain comprises the amino acid sequence VNSDGSHSKGD (SEQ ID NO:5), and CDR3 sequence of the V_{L} domain comprises the amino acid sequence GSSDSSGYV (SEQ ID NO:6).

For example, in some embodiments, the CDR1 sequence of the V_{H} domain comprises the amino acid sequence SDGIS (SEQ ID NO:7), CDR2 sequence of the V_{H} domain comprises the amino acid sequence IISSGGNTYYASWAKG (SEQ ID NO:8), CDR3 sequence of the V_{H} domain comprises the amino acid sequence VVGGTYSI (SEQ ID NO:9), CDR1 sequence of the V_{L} comprises the amino acid sequence QSSQS VYNNDFLS (SEQ ID NO:10), CDR2 sequence of the V_{L} domain comprises the amino acid sequence YASTLAS (SEQ ID NO:11), and CDR3 sequence of the V_{L} domain comprises the amino acid sequence TGTYGNSAWYEDA (SEQ ID NO:12).

For example, in some embodiments, the CDR1 sequence of the V_{H} domain comprises the amino acid sequence SNAMI (SEQ ID NO:13), CDR2 sequence of the V_{H} domain comprises the amino acid sequence AMDSNSRTYYATWAKG (SEQ ID NO:14), CDR3 sequence of the V_{H} domain comprises the amino acid sequence GDGGSSDYTEM (SEQ ID NO:15), CDR1 sequence of the V_{L} comprises the amino acid sequence QSSQSVYGNNELS (SEQ ID NO:16), CDR2 sequence of the V_{L} domain comprises the amino acid sequence QASSLAS (SEQ ID NO:17), and CDR3 sequence of the V_{L} domain comprises the amino acid sequence LGEYSISADNH (SEQ ID NO:18).

In some embodiments, the anti-CD83 scFv V_{H} domain comprises the amino acid sequence:

QVQLKESGPGLVKPSQSLSLTCSVTGFSITTGGYWWTWIRQFPGQKLEWM
GYIFSSGNTNYNPSIKSRISITRDTSKNQFFLQLNSVTTEGDTARYYCAR
AYGKLGFDYWGQGTLVTVSS (SEQ ID NO:19, VH-GBM00).

In some embodiments, the anti-CD83 scFv V_{L} domain comprises the amino acid sequence:

QPVLTQSPSASASLGNSVKITCTLSSQHSTYTIGWYQQHPDKAPKYVMYV
NSDGSHSKGDGIPDRFSGSSSGAHRYLSISNIQPEDEADYFCGSSDSSGY
VFGSGTQLTVL (SEQ IDNO:20, VL-GBM00).

In some embodiments, the anti-CD83 scFv V_{H} domain comprises the amino acid sequence:

METGLRWLLLVAVLKGVQCQSVEESGGRLVTPGTPLTLTCTVSGFSLSNN
AINWVRQAPGKGLEWIGYIWSGGLTYYANWAEGRPTISKTSTTVDLKMTS
PTIEDTATYFCARGINNSALWGPGTLVTVSSGQPKAPSVFPLAPCCGDTP
SSTVTLGCLVKGYLPEPVTVTWNSGTLTNGVRTFPSVRQSSGLYSLSSVV
SVTSSSQPVTCNVAHPATNTKVDKTVAPSTCSKPTCPPPELLGGPSVFIF
PPKPKDTLMISRTPEVTCVVVDVSQDDPEVQFIWYINNEQVRTARPPLRE
QQFNSTIRVVSTLPIAHQDWLRGKEFKCKVHNKALPAPIEKTISKARGQP
LEPKVYTMGPPREELSSRSVSLTCMINGFYPSDISVEWEKNGKAEDNYKT
TPAVLDSDGSYFLYNKLSVPTSEWQRGDVFTCSVMHEALHNHYTQKSISR
SPGK (SEQ ID NO:21,20D04).

In some embodiments, the anti-CD83 scFv V_{L} domain comprises the amino acid sequence:

MDMRAPTQLLGLLLLWLPGARCADVVMTQTPASVSAAVGGTVTINCQASE
SISNYLSWYQQKPGQPPKLLIYRTSTLASGVSSRFKGSGSGTEYTLTISG
VQCDDVATYYCQCTSGGKFISDGAAFGGGTEVVVKGDPVAPTVLLFPPSS
DEVATGTVTIVCVANKYFPDVTVTWEVDGTTQTTGIENSKTPQNSADCTY
NLSSTLTLTSTQYNSHKEYTCKVTQGTTSWQSFSRKNC (SEQ ID NO:
22, 20D04).

In some embodiments, the anti-CD83 scFv V_{H} domain comprises the amino acid sequence:

METGLRWLLLVAVLKGVQCQSVEESGGRLVTPGTPLTLTCTVSGFTISDY
DLSWVRQAPGEGLKYIGFIAIDGNPYYATWAKGRFTISKTSTTVDLKITA
FITEDTATYFCARGAGDLWGPGTLVTVSSGQPKAPSVFPLAPCCGDTPSS
TVTLGCLVKGYLPEPVTVTWNSGTLTNGVRTFPSVRQSSGLYSLSSWSVT
SSSQPVTCNVABPATNTKVDKTVAPSTCSKPTCPPPELLGGPSVFIFPPK
PKDTLMISRTPEVTCVVVDVSQDDPEVQFTWYINNEQVRTARPPLREQQF
NSTIRVVSTLPIAHQDWLRGKEFKCKVHNKALPAPIEKTISKARGQPLEP
KVYTMGPPREELSSRSVSLTCMINGFYPSDISVEWEKNGKAEDNYKTTPA
VLDSDGSYFLYNKLSVPTSEWQRGDVFTCSVMHEALHNHYTQKSISRSPG
K (SEQ ID NO:23,11G05).

In some embodiments, the anti-CD83 scFv V_{L} domain comprises the amino acid sequence:

MDTREPTQLLGLLLLWLPGARCADWMTQTPASVSAAVGGTVTINCQSSKN
VYNNNWLSWFQQKPGQPPKLLIYYASTLASGVPSRFRGSGSGTQFTLTIS
DVQCDDAATYYCAGDYSSSSDNGFGGGTEVVVKGDPVAPTVLLFPPSSDE
VATGTVTIVCVANKYFPDVTVTWEVDGTTQTTGIENSKTPQNSADCTYNL
SSTLTLTSTQYNSHKEYTCKVTQGTTSWQSFSRKNC (SEQ ID NO:24
, 11G05).

In some embodiments, the anti-CD83 scFv V_{H} domain comprises the amino acid sequence:

METGLRWLLLVAVLKGVHCQSVEESGGRLVTPGTPLTLTCTASGFSRSSY
DMSWVRQAPGKGLEWVGVISTAYNSHYASWAKGRFTISRTSTTVDLKMTS
LTTEDTATYFCARGGSWLDLWGQGTLVTVSSGQPKAPSVFPLAPCCGDTP
SSTVTLGCLVKGYLPEPVTVTWNSGTLTNGVRTFPSVRQSSGLYSLSSVV
SVTSSSQPVTCNVAHPATNTKVDKTVAPSTCSKPTCPPPELLGGPSVFIF
PPKPKDTLMISRTPEVTCVVVDVSQDDPEVQFIWYINNEQVRTARPPLRE
QQFNSTIRVVSTLPIAHQDWLRGKEFKCKVHNKALPAPIEKTISKARGQP
LEPKVYTMGPPREELSSRSVSLTCMINGFYPSDISVEWEKNGKAEDNYKT
TPAVLDSDGSYFLYNKLSVFTSEWQRGDVFTCSVMHEALHNHYTQKSISR
SPGK (SEQ IDNO:25, 14C12).

In some embodiments, the anti-CD83 scFv V_{L} domain comprises the amino acid sequence:

MDXRAPTQLLGLLLLWLPGARCALVMTQTPASVSAAVGGTVTINCQSSQS
VYDNDELSWYQQKPGQPPKLLIYALASKLASGVPSRFKGSGSGTQFALTI
SGVQCDDAATYYCQATHYSSDWYLTFGGGTEVVVKGFPVAPTVLLFPPSS
DEVATGTVTIVCVANKYFPDVTVTWEVDGTTQTTGTENSKTPQNSADCTY
NLSSTLTLTSTQYNSHKEYTCKVTQGTTSVVQSFSRKNC (SEQ ID NO
:26, 14C12).

In some embodiments, the anti-CD83 scFv V_{H} domain comprises the amino acid sequence:

METGLRWLLLVAVLKGVQCQSVEESGGRLVTPGTPLTLTCTVSGFSLSSY
DMTWVRQAPGKGLEWIGIIYASGTTYYANWAKGRFTISKTSTTVDLKVTS
PTIGDTATYFCAREGAGVSMTLWGPGTLVTVSSGQPKAPSVFPLAPCCGD
TPSSTVTLGCLVKGYLPEPVTVTWNSGTLTNGVRTFPSVRQSSGLYSLSS
VVSVTSSSQPVTCNVAHPATNTKVDKTVAPSTCSKPTCPPPELLGGPSVF
IFPPKPKDTLMISRTPEVTCVVVDVSQDDPEVQFTWYINNEQVRTARPPL
REQQFNSTIRVVSTLPIAHQDWLRGKEFKCKVHNKALPAPIEKTISKARG
QPLEPKVYTMGPPREELSSRSVSLTCMINGFYPSDISVEWEKNGKAEDNY
KTTPAVLDSDGSYFLYNKLSVPTSEWQRGDVFTCSVMHEALHNHYTQKSI
SRSPGK (SEQ ID NO:27,020B08).

In some embodiments, the anti-CD83 scFv V_{L} domain comprises the amino acid sequence:

MDMRAPTQLLGLLLLWLPGARCAYDMTQTPASVEVAVGGTVTIKCQASQS
ISTYLDWYQQKPGQPPKLLIYDASDLASGVPSRFKGSGSGTQFTLTISDL
ECADAATYYCQQGYTHSNVDNVFGGGTEVVVKGDPVAPTVLLFPPSSDEV
ATGTVTIVCVANKYFPDVTVTWEVDGTTQTTGIENSKTPQNSADCTYNLS
STLTLTSTQYNSHKEYTCKVTQGTTSVVQSFSRKNC (SEQ ID NO:28
, 020B08)

In some embodiments, the anti-CD83 scFv V_{H} domain comprises the amino acid sequence:

METGLRWLLLVAVLKGVQCQSVEESGGRLVSPGTPLTLTCTASGFSLSSY
DMSWVRQAPGKGLEYIGIISSSGSTYYASWAKGRFTISKTSTTVDLEVTS
LTTEDTATYFCSREHAGYSGDTGHLWGPGTLVTVSSGQPKAPSVFPLAPC
CGDTPSSTVTLGCLVKGYLPEPVTVTWNSGTLTNGVRTFPSVRQSSGLYS
LSSVVSVTSSSQPVTCNVAHPATNTKVDKTVAPSTCSKPTCPPPELLGGP
SVGIGPPKPKDTLMISRTPEVTCVVVDVSQDDPEVQFIWYINNEQVRTAR
PPLREQQFNSTIRVVSTLPIAHQDWLRGKEFKCKVHNKALPAPIEKTISK
ARGQPLEPKVYTMGPPREELSSRSVSLTCMINGFYPSDISVEWEKNGKAE
DNYKTTPAVLDSDGSYFLYNKLSVPTSEWQRGDVFTCSVMHEALHNHYTQ
KSISRSPGK (SEQ IDNO:29, 006G05).

In some embodiments, the anti-CD83 scFv V_{L} domain comprises the amino acid sequence:

MDMRAPTQLLGLLLLWLPGARCAYDMTQTPASVEVAVGGTVAIKCQASQS
VSSYLAWYQQKPGQPPKPLIYEASMLAAGVSSRFKGSGSGTDFTLTISDL
ECDDAATYYCQQGYSISDIDNAFGGGTEVVVKGDPVAPTVLLFPPSSDEV
ATGTVTIVCVANKYFPDVTVTWEVDGTTQTTGIENSKTPQNSADCTYNLS
STLTLTSTQYNSHKEYTCKVTQGTTSVVQSFSRKNC (SEQ ID NO:30
, 006G05)

In some embodiments, the anti-CD83 scFv V_{H} domain comprises the amino acid sequence:

METGLRWLLLVAVLKGVQCQSVEESGGRLVTPGTPLTLTCTVSGIDLSSD
GISWVRQAPGKGLEWIGIISSGGNTYYASWAKGRFTISRTSTTVDLKMTS
LTTEDTATYFCARVVGGTYSIWGQGTLVTVSSASTKGPSVYPLAPGSAAQ
TNSMVTLGCLVKGYFPEPVTVTWNSGSLSSGVHTFPAVLQSDLYTLSSSV
TVPSSTWPSETVTCNVAHPASSTKVDKKIVPRDCGCKPCICTVPEVSSVF
IFPPKPDVLTITLTPKVTCVVVDISKDDPEVQFSWFVDDVEVHTAQTQPR
EEQFNSTFRSVSELPIMHQDWLNGKEFKCRVNSAAFPAPIEKTISKTKGR
PKAPQVYTIPPPKEQMAKDKVSLTCMITDFFPEDITVEWQWNGQPAENYK
NTQPIMDTDGSYFVYSKLNVQKSNWEAGNTFTCSVLHEGLHNHHTEKSLS
HSPGK (SEQ IDNO:31, 96G08).

In some embodiments, the anti-CD83 scFv V_{L} domain comprises the amino acid sequence:

MDTRAPTQLLGLLLLWLPGATFAQVLTQTASPVSAPVGGTVTINCQSSQS
VYNNDFLSWYQQKPGQPPKLLIYYASTLASGVPSRFKGSGSGTQFTLTIS
DLECDDAATYYCTGTYGNSAWYEDAFGGGTEVVVKRTPVAPTVLLFPPSS
AELATGTATIVCVANKYFPDGTVTWKVDGITQSSGINNSRTPQNSADCTY
NLSSTLTLSSDEYNSHDEYTCQVAQDSGSPVVQSFSRKSC (SEQ ID N
O:32, 96G08)

In some embodiments, the anti-CD83 scFv V_{H} domain comprises the amino acid sequence:

METGLRWLLLVAVLKGVQCQSVEESGGRLVTPGTPLTLTCTVSGIDLSSN
AMIWVRQAPREGLEWIGAMDSNSRTYYATWAKGRFTISRTSSITVDLKIT
SPTTEDTATYFCARGDGGSSDYTEMWGPGTLVTVSSASTKGPSVYPLAPG
SAAQTNSMVTLGCLVKGYFPEPVTVTWNSGSLSSGVHTFPAVLQSDLYTL
SSSVTVPSSTWPSETVTCNVAHPASSTKVDKKIVPRDCGCKPCICTVPEV
SSVFIFPPKPKDVLTITLTPKVTCVVVDISKDDPEVQFSWFVDDVEVHTA
QTQPREEQFNSTFRSVSELPIMHQDWLNGKEFKCRVNSAAFPAPIEKTIS
KTKGRPKAPQVYTIPPPKEQMAKDKVSLTCMITDFFPEDITVEWQWNGQP
AENYKNTQPIMDTDGSYFVYSKLNVQKSNWEAGNTFTCSVLHEGLHNHHT
EKSLSHSPGK (SEQID NO:33, 95F04).

In some embodiments, the anti-CD83 scFv V_{L} domain comprises the amino acid sequence:

MDTRAPTQLLGLLLLWLPGATFAQAVVTQTTSPVSAPVGGTVTINCQSSQ
SVYGNNELSWYQQKPGQPPKLLIYQASSLASGVPSRFKGSGSGTQFTLTI
SDLECDDAATYYCLGEYSISADNHFGGGTEVVVKRTPVAPTVLLFPPSSA
ELATGTATIVCVANKYFPDGTVTWKVDGITQSSGINNSRTPQNSADCTYN
LSSTLTLSSDEYNSHDEYTCQVAQDSGSPVVQSFSRKSC (SEQ ID NO
:34, 95F04)

In some embodiments, the anti-CD83 scFv V_{H} domain comprises the amino acid sequence:

QVQLVQSGGAVVQPGRSLRLSCAASGFTFSTYGMHWVRQAPGKGLEWVAA
VSYDGSNKYYADFVKGRFTISRDNPKNTLYLQMNSLRADDTAVYYCARRG
GLDIWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNV
NHKPSNTKVDKKVEPKSCAAA (SEQ IDNO:35).

In some embodiments, the anti-CD83 scFv V_{L} domain comprises the amino acid sequence:

LTQPPPASGTPGQQRVTISCSGSSSNIGSNTVNWYQQLPGTAPKLLIYYG
NDQRPSGVPDRFSASKSGTSASLAISGLQSEDEAHYYCAAWDGSLNGGVI
FGGGTKVTLG (SEQ IDNO:36).

In some embodiments, the anti-CD83 scFv V_{L} domain comprises the amino acid sequence:

VTQPPSASGTPGQRVTISCSGSSSNIGTNPVNWYQQLPGTAPKLLIYTTD
QRPSGVPDRFSGSKSGTSASLAISGLQSEDEADYYCAAWDDSLSGLYVFG
TGTKVTVLG (SEQ IDNO:37).

In some embodiments, the anti-CD83 scFv VL domain comprises the amino acid sequence:

MTHTPLSLSVTPGQPASISCKSSQSLLHSDGKTYLYWYLQRPGQSPQPLI
YEVSNRFSGVPDRFSGSGSGTDFTLKISRVQAEDVGVYYCMQSLQLWTFG
QGTKVEIKR (SEQ IDNO:38).

In some embodiments, the anti-CD83 scFv V_{L} domain comprises the amino acid sequence:

MTQSPLSLPVTLGQPASISCRSSQSLIHSDGNTYLDWFQQRPGQSPRRLI
YKVSNRDSGVPDRFSGSGSGTDFTLRISRVEAEDIGVYYCMQATHWPRTF
GQGTKVEIKR (SEQ IDNO:39).

In some embodiments, the anti-CD83 scFv V_{L} domain comprises the amino acid sequence:

MTQSPLSLPVTLGQPASISCRSSQSLVDSAGNTFLHWFHQRPGQSPRRLI
YKVSNRDSGVPDRFSGSGSGTDFTLKISRVEAEDVGVYYCMQGTHWPRTF
GQGTKVEIKR (SEQ IDNO:40).

In some embodiments, the anti-CD83 scFv V_{L} domain comprises the amino acid sequence:

LTQSPLSLPVTLGQPASISCKSSQSLVDSDGNTYLNWFQQRPGQSPRRLI
YKVSNRDSGVPDRFSGSGSGTDFTLKISRVEAEDVGVYYCMQGTHWPRTF
GQGTKVEIKR (SEQ IDNO:41).

In some embodiments, the anti-CD83 scFv V_{L} domain comprises the amino acid sequence:

MTQSPLSLPVTLGQPASISCRSSQSLVHSDGNMYLNWFQQRPGQSPRRLI
YKVSNRDSGVPDRFSGSGSGTDFTLKISRVEAEDVGVYYCMQATQPTWTF
GQGTKLEIKR (SEQ IDNO:42).

In some embodiments, the anti-CD83 scFv V_{L} domain comprises the amino acid sequence:

MTQSPSSLSASVGDRVTITCQASQDISNYLNWYQQKPGKAPKLLIYDASN
LETGVPSRFSGSGSGTDFTFTISSATYYCQQTYQGTKLEIKR (SEQ ID
NO:43).

In some embodiments, the anti-CD83 scFv V_{L} domain comprises the amino acid sequence:

MTQSPSSLSASVGHPVTITCRASQSLISYLNWYHQKPGKAPKLLIYAASI
LQSGVPSRFSGSGSGTDFTLTISSLQPENFASYYCQHTDSFPRTFGHGTK
VEIKR (SEQ ID NO:44).

In some embodiments, the anti-CD83 scFv V_{L} domain comprises the amino acid sequence:

LTQPPSASGTPGQGVTISCRGSTSNIGNNVVNWYQHVPGSAPKLLIWSNI
QRPSGIPDRFSGSKSGTSASLAISGLQSEDQAVYYCAVWDDGLAGWVFGG
GTTVTVLS (SEQ IDNO:45).

In some embodiments, the anti-CD83 scFv V_{L} domain comprises the amino acid sequence:

MTQAPVVSVALEQTVRITCQGDSLAIYYDFWYQHKPGQAPVLVIYGKNNR
PSGIPHRFSGSSSNTDSLTITGAQAEDEADYYCNSRDSSGNHWVFGGGTN
LTVLG (SEQ IDNO:46).

In some embodiments, the anti-CD83 scFv V_{L} domain comprises the amino acid sequence:

LTQSPLSLPVTLGQPASISCKSNQSLVHSDGNTYLNWFQQRPGQSPRRLI
YKVSNRDSGVPDRFSGSGSGTDFTLKINRVEAEDVGVYYCMQGTQWPRTF
GGQGTKLDIKR(SEQ IDNO:47).

In some embodiments, the anti-CD83 scFv V_{H} domain has been humanized and comprises the amino acid sequence:

QVQLQESGPGLVKPSETLSLTCTVSGFSITTGGYWWTWIRQPPGKGLEWI
GYIFSSGNTNYNPSIKSRVTISVDTSKNQFSLKLSSVTAADTAVYYCARA
YGKLGFDYWGQGTLVTVSS (SEQ ID NO:48, VH-GBM01).

In some embodiments, the anti-CD83 scFv V_{H} domain has been humanized and comprises the amino acid sequence:

QVQLQESGPGLVKPSQTLSLTCTVSGFSITTGGYWWTWIRQHPGKGLEWI
GYIFSSGNTNYNPSIKSLVTISVDTSKNQFSLKLSSVTAADTAVYYCARA
YGKLGFDYWGQGTLVTVSS (SEQ ID NO:49, VH-GBM02).

In some embodiments, the anti-CD83 scFv V_{H} domain has been humanized and comprises the amino acid sequence:

QVQLQESGPGLVKPSQTLSLTCTVSGFSITTGGYWWTWIRQPPGKGLEWI
GYIFSSGNTNYNPSIKSRVTISVDTSKNQFSLKLSSVTAADTAVYYCARA
YGKLGFDYWGQGTLVTVSS (SEQ ID NO:50, VH-GBM03).

In some embodiments, the anti-CD83 scFv V_{H} domain has been humanized and comprises the amino acid sequence:

QVQLQESGPGLVKPSETLSLTCTVSGFSITTGGYWWTWIRQPPGKGLEWI
GYIFSSGNTNYNPSIKSRVTISRDTSKNQFSLKLSSVTAADTAVYYCARA
YGKLGFDYWGQGTLVTVSS (SEQ ID NO:51, VH-GBM04).

In some embodiments, the anti-CD83 scFv V_{H} domain has been humanized and comprises the amino acid sequence:

QVQLQESGPGLVKPSETLSLTCTVSGFSITTGGYWWTWIRQPPGKGLEWI
GYIFSSGNTNYNPSIKSRVTISVDTSKNQFSLKLSSVTAADTARYYCARA
YGKLGFDYWGQGTLVTVSS (SEQ ID NO:52, VH-GBM05).

In some embodiments, the anti-CD83 scFv V_{H} domain has been humanized and comprises the amino acid sequence:

QVQLQESGPGLVKPSETLSLTCTVSGFSITTGGYWWTWIRQPPGKGLEWI
GYIFSSGNTNYNPSIKSRISITRDTSKNQFFLQLNSVTTEGDTARYYCAR
AYGKLGFDYWGQGTLVTVSS (SEQ ID NO:53, VH-GBM06).

In some embodiments, the anti-CD83 scFv V_{L} domain has been humanized and comprises the amino acid sequence:

QLVLTQSPSASASLGASVKLTCTLSSQHSTYTIGWHQQQPEKGPRYLMKV
NSDGSHSKGDGIPDRFSGSSSGAERYLTISSLQSEDEADYYCGSSDSSGY
VFGSGTKVTVL (SEQ IDNO:54, VL-GBM01).

In some embodiments, the anti-CD83 scFv V_{L} domain has been humanized and comprises the amino acid sequence:

LPVLTQPPSASALLGASIKLTCTLSSQHSTYTIGWYQQRPGRSPQYIMKV
NSDGSHSKGDGIPDRFMGSSSGADRYLTFSNLQSDDEAEYHCGSSDSSGY
VFGSGTKVTVL (SEQ IDNO:55, VL-GBM02).

The heavy and light chains are preferably separated by a linker. Suitable linkers for scFv antibodies are known in the art. In some embodiments, the linker comprises the amino acid sequence GGGGSGGGGSGGGGS (SEQ ID NO:56).

In some embodiments, the anti-CD83 scFv comprises an amino acid sequence:

QPVLTQSPSASASLGNSVKITCTLSSQHSTYTIGWYQQHPDKAPKYVMY
VNSDGSHSKGDGIPDRFSGSSSGAHRYLSISNIQPEDEADYFCGSSDSSG
YVFGSGTQLTVLRAAASSGGGGSGGGGSGGGGSQPVLTQSPSASASLGNS
VKITCTLSSQHSTYTIGWYQQHPDKAPKYVMYVNSDGSHSKGDGIPDRFS
GSSSGAHRYLSISNIQPEDEADYFCGSSDSSGYVFGSGTQLTVLRAAA (
SEQ ID NO:57).

In some embodiments, the anti-CD83 scFv comprises an amino acid sequence:

QVQLKESGPGLVKPSQSLSLTCSVTGFSITTGGYWWTWIRQFPGQKLEW
MGYIFSSGNTNYNPSIKSRISITRDTSKNQFFLQLNSVTTEGDTARYYCA
RAYGKLGFDYWGQGTLVTVSSGGGGSGGGGSGGGGSQVQLKESGPGLVKP
SQSLSLTCSVTGFSITTGGYWWTWIRQFPGQKLEWMGYIFSSGNTNYNPS
IKSRISITRDTSKNQFFLQLNSVTTEGDTARYYCARAYGKLGFDYWGQGT
LVTV (SEQ ID NO:58).

In some embodiments, the anti-CD83 scFv comprises an amino acid sequence:

QVQLQESGPGLVKPSETLSLTCTVSGFSITTGGYWWTWIRQPPGKGLEWI
GYIFSSGNTNYNPSIKSRVTISVDTSKNQFSLKLSSVTAADTAVYYCARA
YGKLGFDYWGQGTLVTVSSGGGGSGGGGSGGGGSQLVLTQSPSASASLGA
SVKLTCTLSSQHSTYTIGWHQQQPEKGPRYLMKVNSDGSHSKGDGIPDRF
SGSSSGAERYLTISSLQSEDEADYYCGSSDSSGYVFGSGTKVTVL (SEQ
ID NO:59).

In some embodiments, the anti-CD83 scFv comprises an amino acid sequence:

QVQLQESGPGLVKPSQTLSLTCTVSGFSITTGGYWWTWIRQHPGKGLEWI
GYIFSSGNTNYNPSIKSLVTISVDTSKNQFSLKLSSVTAADTAVYYCARA
YGKLGFDYWGQGTLVTVSSGGGGSGGGGSGGGGSQLVLTQSPSASASLGA
SVKLTCTLSSQHSTYTIGWHQQQPEKGPRYLMKVNSDGSHSKGDGIPDRF
SGSSSGAERYLTISSLQSEDEADYYCGSSDSSGYVFGSGTKVTVL (SEQ
ID NO:60.

In some embodiments, the anti-CD83 scFv comprises an amino acid sequence:

QVQLQESGPGLVKPSQTLSLTCTVSGFSITTGGYWWTWIRQPPGKGLEWI
GYIFSSGNTNYNPSIKSRVTISVDTSKNQFSLKLSSVTAADTAVYYCARA
YGKLGFDYWGQGTLVTVSSGGGGSGGGGSGGGGSQLVLTQSPSASASLGA
SVKLTCTLSSQHSTYTIGWHQQQPEKGPRYLMKVNSDGSHSKGDGIPDRF
SGSSSGAERYLTISSLQSEDEADYYCGSSDSSGYVFGSGTKVTVL (SEQ
ID NO:61).

In some embodiments, the anti-CD83 scFv comprises an amino acid sequence:

QVQLQESGPGLVKPSETLSLTCTVSGFSITTGGYWWTWIRQPPGKGLEWI
GYIFSSGNTNYNPSIKSRVTISRDTSKNQFSLKLSSVTAADTAVYYCARA
YGKLGFDYWGQGTLVTVSSGGGGSGGGGSGGGGSQLVLTQSPSASASLGA
SVKLTCTLSSQHSTYTIGWHQQQPEKGPRYLMKVNSDGSHSKGDGIPDRF
SGSSSGAERYLTISSLQSEDEADYYCGSSDSSGYVFGSGTKVTVL (SEQ
ID NO:62).

In some embodiments, the anti-CD83 scFv comprises an amino acid sequence:

QVQLQESGPGLVKPSETLSLTCTVSGFSITTGGYWWTWIRQPPGKGLEWI
GYIFSSGNTNYNPSIKSRVTISVDTSKNQFSLKLSSVTAADTARYYCARA
YGKLGFDYWGQGTLVTVSSGGGGSGGGGSGGGGSQLVLTQSPSASASLGA
SVKLTCTLSSQHSTYTIGWHQQQPEKGPRYLMKVNSDGSHSKGDGIPDRF
SGSSSGAERYLTISSLQSEDEADYYCGSSDSSGYVFGSGTKVTVL (SEQ
ID NO:63).

In some embodiments, the anti-CD83 scFv comprises an amino acid sequence:

QVQLQESGPGLVKPSETLSLTCTVSGFSITTGGYWWTWIRQPPGKGLEWI
GYIFSSGNTNYNPSIKSRISITRDTSKNQFFLQLNSVTTEGDTARYYCAR
AYGKLGFDYWGQGTLVTVSSGGGGSGGGGSGGGGSQLVLTQSPSASASLG
ASVKLTCTLSSQHSTYTIGWHQQQPEKGPRYLMKVNSDGSHSKGDGIPDR
FSGSSSGAERYLTISSLQSEDEADYYCGSSDSSGYVFGSGTKVTVL (SE
Q ID NO:64).

In some embodiments, the anti-CD83 scFv comprises an amino acid sequence:

QVQLQESGPGLVKPSETLSLTCTVSGFSITTGGYWWTWIRQPPGKGLEWI
GYIFSSGNTNYNPSIKSRVTISVDTSKNQFSLKLSSVTAADTAVYYCARA
YGKLGFDYWGQGTLVTVSSGGGGSGGGGSGGGGSLPVLTQPPSASALLGA
SIKLTCTLSSQHSTYTIGWYQQRPGRSPQYIMKVNSDGSHSKGDGIPDRF
MGSSSGADRYLTFSNLQSDDEAEYHCGSSDSSGYVFGSGTKVTVL (SEQ
ID NO:65).

In some embodiments, the anti-CD83 scFv comprises an amino acid sequence:

QVQLQESGPGLVKPSQTLSLTCTVSGFSITTGGYWWTWIRQHPGKGLEWI
GYIFSSGNTNYNPSIKSLVTISVDTSKNQFSLKLSSVTAADTAVYYCARA
YGKLGFDYWGQGTLVTVSSGGGGSGGGGSGGGGSLPVLTQPPSASALLGA
SIKLTCTLSSQHSTYTIGWYQQRPGRSPQYIMKVNSDGSHSKGDGIPDRF
MGSSSGADRYLTFSNLQSDDEAEYHCGSSDSSGYVFGSGTKVTVL (SEQ
ID NO:66).

In some embodiments, the anti-CD83 scFv comprises an amino acid sequence:

QVQLQESGPGLVKPSQTLSLTCTVSGFSITTGGYWWTWIRQPPGKGLEWI
GYIFSSGNTNYNPSIKSRVTISVDTSKNQFSLKLSSVTAADTAVYYCARA
YGKLGFDYWGQGTLVTVSSGGGGSGGGGSGGGGSLPVLTQPPSASALLGA
SIKLTCTLSSQHSTYTIGWYQQRPGRSPQYIMKVNSDGSHSKGDGIPDRF
MGSSSGADRYLTFSNLQSDDEAEYHCGSSDSSGYVFGSGTKVTVL (SEQ
ID NO:67).

In some embodiments, the anti-CD83 scFv comprises an amino acid sequence:

QVQLQESGPGLVKPSETLSLTCTVSGFSITTGGYWWTWIRQPPGKGLEWI
GYIFSSGNTNYNPSIKSRVTISRDTSKNQFSLKLSSVTAADTAVYYCARA
YGKLGFDYWGQGTLVTVSSGGGGSGGGGSGGGGSLPVLTQPPSASALLGA
SIKLTCTLSSQHSTYTIGWYQQRPGRSPQYIMKVNSDGSHSKGDGIPDRF
MGSSSGADRYLTFSNLQSDDEAEYHCGSSDSSGYVFGSGTKVTVL (SEQ
ID NO:68).

In some embodiments, the anti-CD83 scFv comprises an amino acid sequence:

QVQLQESGPGLVKPSETLSLTCTVSGFSITTGGYWWTWIRQPPGKGLEWI
GYIFSSGNTNYNPSIKSRVTISVDTSKNQFSLKLSSVTAADTARYYCARA
YGKLGFDYWGQGTLVTVSSGGGGSGGGGSGGGGSLPVLTQPPSASALLGA
SIKLTCTLSSQHSTYTIGWYQQRPGRSPQYIMKVNSDGSHSKGDGIPDRF
MGSSSGADRYLTFSNLQSDDEAEYHCGSSDSSGYVFGSGTKVTVL (SEQ
ID NO:69).

In some embodiments, the anti-CD83 scFv comprises an amino acid sequence:

QVQLQESGPGLVKPSETLSLTCTVSGFSITTGGYWWTWIRQPPGKGLEWI
GYIFSSGNTNYNPSIKSRISITRDTSKNQFFLQLNSVTTEGDTARYYCAR
AYGKLGFDYWGQGTLVTVSSGGGGSGGGGSGGGGSLPVLTQPPSASALLG
ASIKLTCTLSSQHSTYTIGWYQQRPGRSPQYIMKVNSDGSHSKGDGIPDR
FMGSSSGADRYLTFSNLQSDDEAEYHCGSSDSSGYVFGSGTKVTVL (SE
Q ID NO:70).

In some embodiments, the anti-CD83 scFv comprises an amino acid sequence:

QVQLKESGPGLVKPSQSLSLTCSVTGFSITTGGYWWTWIRQFPGQKLEWM
GYIFSSGNTNYNPSIKSRISITRDTSKNQFFLQLNSVTTEGDTARYYCAR
AYGKLGFDYWGQGTLVTVSSGGGGSGGGGSGGGGSQPVLTQSPSASASLG
NSVKITCTLSSQHSTYTIGWYQQHPDKAPKYVMYVNSDGSHSKGDGIPDR
FSGSSSGAHRYLSISNIQPEDEADYFCGSSDSSGYVFGSGTQLTVL (SE
Q ID NO:71).

In some embodiments, the anti-CD83 CAR disclosed herein comprises the amino acid sequence:

QVQLKESGPGLVKPSQSLSLTCSVTGFSITTGGYWWTWIRQFPGQKLEWM
GYIFSSGNTNYNPSIKSRISITRDTSKNQFFLQLNSVITEGDTARYYCAR
AYGKLGFDYWGQGTLVTVSSGGGGSGGGGSGGGGSQPVLTQSPSASASLG
NSVKITCTLSSQHSTYTIGWYQQHPDKAPKYVMYVNSDGSHSKGDGIPDR
FSGSSSGAHRYLSISNIQPEDEADYFCGSSDSSGYVFGSGTQLTVLRAAA
TITPAPRPFTPAFFIASQPLSLRPEACRPAAGGAVHTRGLDFACDIYIWA
PLAGTCGVLLLSLVITLYCKRGRKKLLYIFKQPFMRPVQTTQEEDGCSCR
FPEEEEGGCELRVKFSRSADAPAYKQGQNQLYNELNLGRREEYDVLDKRR
GRDPEMGGKPRRKNPQEGLYNELQKDKMAEAYSEIGMKGERRRGKGHDGL
YQGLSTATKDTYDALHMQALPPR (SEQ ID NO: 90).

In some examples, the anti-CD83 CAR may further comprise a signal peptide at the N-terminus. One example include MALPVTALLLPLALLLHAARP (SEQ ID NO:91)

### Dual CAR System Targeting Both CD83 and CD19

Also disclosed herein is a CD19-specific CAR (anti-CD19 CAR) that can be co-expressed with an anti-CD83 CAR in immune cells. In some embodiments, the immune cells expressing both an anti-CD83 CAR and an anti-CD19 CAR may selectively suppress alloreactive or autoreactive lymphocytes. For example, the endodomain of each CAR contains an SD or a CSR, but not both. In these embodiments, an immune cell containing the disclosed CARs is only activated if both the anti-CD19 and anti-CD83 CARs bind their respective antigens. For example, in some embodiments, the disclosed anti-CD83 and anti-CD19 CARs are defined by the formulas:

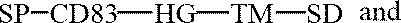

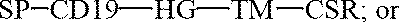

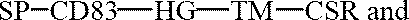

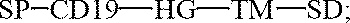

wherein “SP” represents an optional signal peptide,
wherein “CD83” represents a CD83-binding region,
wherein “CD19” represents a CD19-binding region,
wherein “HG” represents an optional hinge domain,
wherein “TM” represents a transmembrane domain,
wherein “CSR” represents one or more co-stimulatory signaling regions,
wherein “SD” represents a signaling domain, and
wherein “-” represents a peptide bond or linker.

In other embodiments, the immune cells expressing both an anti-CD83 CAR and an anti-CD19 CAR may suppress alloreative or autoreactive lymphocytes that expressing either CD83 or CD19. In some examples, each of the anti-CD83 and anti-CD19 CARs can comprise at least an ectodomain specific to CD83 or CD19, a co-stimulatory signaling domain as those disclosed herein, and a cytoplasmic signaling domain such as Cd3□. Each CAR may further comprise a transmembrane domain and optionally a hinge domain.

In some embodiments, the anti-CD19 scFv can comprise a variable heavy (V_{H}) domain having CDR1, CDR2 and CDR3 sequences and a variable light (V_{L}) domain having CDR1, CDR2 and CDR3 sequences.

In some embodiments, the anti-CD19 scFv comprises an scFv described in US20160145337, which is incorporated by reference for the teaching of this antibody. For example, in some embodiments, the CDR1 sequence of the VH domain comprises the amino acid sequence SYWMN (SEQ ID NO:72), CDR2 sequence of the VH domain comprises the amino acid sequence QIWPGDGDTNYNGKFKG (SEQ ID NO:73), CDR3 sequence of the VH domain comprises the amino acid sequence RETTTVGRYYYAMDY (SEQ ID NO:74), CDR1 sequence of the VL comprises the amino acid sequence KASQSVDYDGDSYLN (SEQ ID NO:75), CDR2 sequence of the VL domain comprises the amino acid sequence DASNLVS (SEQ ID NO:76), and CDR3 sequence of the VL domain comprises the amino acid sequence QQSTEDPWT (SEQ ID NO:77). In some embodiments, the anti-CD19 scFv V_{H} domain has been humanized and comprises the amino acid sequence: QVQLQESGAELVRPGSSVKISCKASGYAFSSYWMNWVKQRPGQGLEWIGQIWPGDG DTNYNGKFKGKATLTADESSSTAYMQLSSLRSEDSAVYSCARRETTTVGRYYYAMD YWGQGTTVTVSS (SEQ ID NO:78). In some embodiments, the anti-CD19 scFv V_{L} domain has been humanized and comprises the amino acid sequence:

DIQLTQSPASLAVSLGQRATISCKASQSVDYDGDSYLNWYQQIPGQPPKI
LLIYDASNLVSGIPPRFSGSGSGTDFTLNIHPVEKVDAATYHCQQSTEDP
WTFGGGTKLEIKR (SEQ IDNO:79).

In some embodiments, the anti-CD19 scFv comprises an scFv described in WO2016168773, which is incorporated by reference for the teaching of this antibody. In some embodiments, the anti-CD19 scFv V_{H} domain has been humanized and comprises the amino acid sequence:

EVKLQESGPGLVAPSQSLSVTCTVSGVSLPDYGVSWIRQPPRKGLEWLGV
IWGSETTYYNSALKSRLTIIKDNSKSQVFLKMNSLQTDDTAIYYCAKHYY
YGGSYAMDYWGQGTSVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSC (SEQ ID

NO:80). In some embodiments, the anti-CD19 scFv V_{L} domain has been humanized and comprises the amino acid sequence:

DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEIKRTVAAPSVFIFPPSDEQLKSGTASVVCLLNNFYPREAKVQWKV
DNALQSGNSQESVTEQDSGGGGSNYHLENEVARLKKLGGGGSDSTYSLSS
TLTLSKADYEKHKVYACEVT (SEQ ID NO:81). In

some embodiments, the anti-CD19 scFv V_{L} domain has been humanized and comprises the amino acid sequence:

EVKLQESGPGLVAPSQSLSVTCTVSGVSLPDYGVSWIRQPPRKGLEWLGV
IWGSETTYYNSALKSRLTIIKDNSKSQVFLKMNSLQTDDTAIYYCAKHYY
YGGSYAMDYWGQGTSVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSC (SEQ IDNO:82).

In some embodiments, the anti-CD19 scFv comprises an scFv described in US20160039942, which is incorporated by reference for the teaching of this antibody. In some embodiments, the anti-CD19 scFv V_{H} domain has been humanized and comprises the amino acid sequence: QVQLQQSGAELVRPGSSVKISCKASGYAFSSYWMNWVKQRPGQGLEWIGQIWPGDG DTNYNGKFKGKATLTADESSSTAYMQLSSLASEDSAVYFCARRETTTVGRYYYAMD YWGQGTTVTVSS (SEQ ID NO:83). In some embodiments, the anti-CD19 scFv V_{L} domain has been humanized and comprises the amino acid sequence:

DIQLTQSPASLAVSLGQRATISCKASQSVDYDGDSYLNWYQQIPGQPPKI
LLIYDASNLVSGIPPRFSGSGSGTDFTLNIHPVEKVDAATYHCQQSTEDP
WTFGGGTKLEIK (SEQ IDNO:84).

In some embodiments, the anti-CD19 scFv comprises an scFv described in WO2014184143, which is incorporated by reference for the teaching of this antibody. In some embodiments, the anti-CD19 scFv V_{H} domain has been humanized and comprises the amino acid sequence:

MEWSWIFLFLLSGTAGVHSEVQLQQSGPELIKPGASVKMSCKASGYTFTS
YVMHWVKQKPGQGLEWIGYINPYNDGTKYNEKFKGKATLTSDKSSSTAYM
ELSSLTSEDSAVYYCARGTYYYGSRVFDYWGQGTTLTVSSAKTTPPSVYP
LAPGSAAQTNSMVTLGCLVKGYFPEPVTVTWNSGSLSSGVHTFPAVLQSD
LYTLSSSVTVPSSTWPSETVTCNVAHPASSTKVDKKIVPRDCGCKPCICT
VPEVSSVFIFPPKPKDVLTITLTPKVTCVVVDISKDDPEVQFSWFVDDVE
VHTAQTQPREEQFNSTFRSVSELPIMHQDWLNGKEFKCRVNSAAFPAPIE
KTISKTKGRPKAPQVYTIPPPKEQMAKDKVSLTCMITDFFPEDITVEWQW
NGQPAENYKNTQPIMDTDGSYFVYSKLNVQKSNWEAGNTFTCSVLHEGLH
NHHTEKSLSHSPGK

(SEQ ID NO:85). In some embodiments, the anti-CD19 scFv V_{H} domain has been humanized and comprises the amino acid sequence: EVQLQQSGPELIKPGASVKMSCKASGYTFTSYVMHWVKQKPGQGLEWIGYINPYNDG TKYNEKFKGKATLTSDKSSSTAYMELSSLTSEDSAVYYCARGTYYYGSRVFDYWGQG TTLTVSS (SEQ ID NO:86). In some embodiments, the anti-CD19 scFv V_{L} domain has been humanized and comprises the amino acid sequence: MRCLAEFLGLLVLWIPGAIGDIVMTQAAPSIPVTPGESVSISCRSSKSLLNSNGNTYLY WFLQRPGQSPQLLIYRMSNLASGVPDRFSGSGSGTAFTLRISRVEAEDVGVYYCMQHL EYPFTFGAGTKLELKRADAAPTVSIFPPSSEQLTSGGASVVCFLNNFYPKDINVKWKID GSERQNGVLNSWTDQDSKDSTYSMSSTLTLTKDEYERHNSYTCEATHKTSTSPIVKSF NRNEC (SEQ ID NO:87). In some embodiments, the anti-CD19 scFv V_{L} domain has been humanized and comprises the amino acid sequence:

DIVMTQAAPSIPVTPGESVSISCRSSKSLLNSNGNTYLYWFLQRPGQSPQ
LLIYRMSNLASGVPDRFSGSGSGTAFTLRISRVEAEDVGVYYCMQHLEYP
FTFGAGTKLELKRAD

(SEQ ID NO:88). In some embodiments, the anti-CD19 scFv V_{L} domain has been humanized and comprises the amino acid sequence:

DIVMTQAAPSIPVTPGESVSISCRSSKSLLNSNGNTYLYWFLQRPGQSPQ
LLIYRMSNLASGVPDRFSGSGSGTAFTLRISRVEAEDVGVYYCMQHLEYP
FTFGAGTKLELKRSDP(SEQ ID NO:89).

In some embodiments, the anti-CD19 scFv comprises an antigen binding domain of clone FMC63, described in Hohmann, A.W. Mol. Immunol. 34(16-17):1157-1165, which is incorporated by reference for the teaching of this antibody.

In some embodiments, the CAR is a CAR described in Nicholson et al., Molecular Immunology, 34(16-17): 1157-1165 (1997), which is incorporated by reference for the teaching of CARs containing FMC63 anti-CD19 scFvs.

In some embodiments, the anti-CD19 scFv comprises the FVS191 or FVS192 scFv describe in Bejcek BE, et al. Cancer Res. 1995 55(11):2346-51, which is incorporated by reference for the teaching of this antibody.

In some embodiments, the anti-CD19 scFv comprises an antigen binding domain of clone SJ25C1, B43, B4, AB1, BU12, F974A2, HD37, SJ25-C1, HIB19, or 4G7.

In some embodiments, any of the CARs disclosed herein can be a TRUCK, Universal CAR, Self-driving CAR, Armored CAR, Self-destruct CAR, Conditional CAR, Marked CAR, TenCAR, Dual CAR, or sCAR.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

A tandem CAR (TanCAR) T cell expresses a single CAR consisting of two linked single-chain variable fragments (scFvs) that have different affinities fused to intracellular co-stimulatory domain(s) and a CD3ζ domain. TanCAR T cell activation is achieved only when target cells co-express both targets.

A dual CAR T cell expresses two separate CARs with different ligand binding targets; one CAR includes only the CD3□ domain and the other CAR includes only the co-stimulatory domain(s). Dual CAR T cell activation requires co-expression of both targets.

A safety CAR (sCAR) consists of an extracellular scPv fused to an intracellular inhibitory domain. sCAR T cells co-expressing a standard CAR become activated only when encountering target cells that possess the standard CAR target but lack the sCAR target.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune cell, activating at least one of the normal effector functions of the immune cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (Fc gamma RIIa), DAP10, DAP12, CD79a, CD79b, FcγRIγ, FcγRIIIγ, FcεRIβ (FCERIB), and FcεRIγ (FCERIG).

In particular embodiments, the intracellular signaling domain is derived from CD3 zeta (CD3ζ) (TCR zeta, GenBank accno. BAG36664.1). T-cell surface glycoprotein CD3 zeta (CD3ζ) chain, also known as T-cell receptor T3 zeta chain or CD247 (Cluster of Differentiation 247), is a protein that in humans is encoded by the CD247 gene.

First-generation CARs typically had the intracellular domain from the CD3ζ chain, which is the primary transmitter of signals from endogenous TCRs. Second-generation CARs add intracellular signaling domains from various costimulatory protein receptors (e.g., CD28, 41BB, ICOS) to the endodomain of the CAR to provide additional signals to the T cell. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD3ζ signaling domain by itself or combined with any other desired cytoplasmic domain(s) useful in the context of the CAR of the invention. For example, the cytoplasmic domain of the CAR can comprise a CD3ζ chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4–1BB (CD137), OX40, CD30, CD40, ICOS, lymphocyte function-associated antigen-1 (LFA-1), CD2, CD7, LIGHT, NKG2C, B7-H3, and a ligand that specifically binds with CD123, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat Rev. Immunol., 4(2): 89-99 (2004)). The hinge sequence may be positioned between the antigen recognition moiety (e.g., anti-CD83 scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

Tables 1, 2, and 3 below provide some example combinations of CD83-binding region, co-stimulatory signaling regions, and intracellular signaling domain that can occur in the disclosed CARs.

TABLE 1

First Generation CARs

| | |
|---|---|
| ScFv | Signal Domain |
| CD83 | CD8 |
| CD83 | CD3ζ |
| CD83 | CD3δ |
| CD83 | CD3γ |
| CD83 | CD3ε |
| CD83 | FcγRI-γ |
| CD83 | FcγRIII-γ |
| CD83 | FcεRIβ |
| CD83 | FcεRIγ |
| CD83 | DAP10 |
| CD83 | DAP12 |
| CD83 | CD32 |
| CD83 | CD79a |

TABLE 2

Second Generation CARs

| | | | | | |
|---|---|---|---|---|---|
| ScFv | Co-stimulatory Signal | Signal Domain | ScFv | Co-stimulatory Signal | Signal Domain |
| CD83 | CD28 | CD8 | CD83 | CD80 | FcεRIβ |
| CD83 | CD28 | CD3ζ | CD83 | CD80 | FcεRIγ |
| CD83 | CD28 | CD3δ | CD83 | CD80 | DAP10 |
| CD83 | CD28 | CD3γ | CD83 | CD80 | DAP12 |
| CD83 | CD28 | CD3ε | CD83 | CD80 | CD32 |
| CD83 | CD28 | FcγRI-γ | CD83 | CD80 | CD79a |
| CD83 | CD28 | FeγRIII-γ | CD83 | CD80 | CD79b |
| CD83 | CD28 | FcεRIβ | CD83 | CD86 | CD8 |
| CD83 | CD28 | FcεRIγ | CD83 | CD86 | CD3ζ |
| CD83 | CD28 | DAP10 | CD83 | CD86 | CD3δ |
| CD83 | CD28 | DAP12 | CD83 | CD86 | CD3γ |
| CD83 | CD28 | CD32 | CD83 | CD86 | CD3ε |
| CD83 | CD28 | CD79a | CD83 | CD86 | FcγRI-γ |
| CD83 | CD28 | CD79b | CD83 | CD86 | FcγRIII-γ |
| CD83 | CD8 | CD8 | CD83 | CD86 | FcεRIβ |
| CD83 | CD8 | CD3ζ | CD83 | CD86 | FcεRIγ |
| CD83 | CD8 | CD3δ | CD83 | CD86 | DAP10 |
| CD83 | CD8 | CD3γ | CD83 | CD86 | DAP12 |
| CD83 | CD8 | CD3ε | CD83 | CD86 | CD32 |
| CD83 | CD8 | FcγRI-γ | CD83 | CD86 | CD79a |
| CD83 | CD8 | FeγRIII-γ | CD83 | CD86 | CD79b |
| CD83 | CD8 | FcεRIβ | CD83 | OX40 | CD8 |
| CD83 | CD8 | FcεRIγ | CD83 | OX40 | CD3ζ |
| CD83 | CD8 | DAP10 | CD83 | OX40 | CD3δ |
| CD83 | CD8 | DAP12 | CD83 | OX40 | CD3γ |
| CD83 | CD8 | CD32 | CD83 | OX40 | CD3ε |
| CD83 | CD8 | CD79a | CD83 | OX40 | FcγRI-γ |
| CD83 | CD8 | CD79b | CD83 | OX40 | FeγRIII-γ |
| CD83 | CD4 | CD8 | CD83 | OX40 | FcεRIβ |
| CD83 | CD4 | CD3ζ | CD83 | OX40 | FcεRIγ |
| CD83 | CD4 | CD3δ | CD83 | OX40 | DAP10 |
| CD83 | CD4 | CD3γ | CD83 | OX40 | DAP12 |
| CD83 | CD4 | CD3ε | CD83 | OX40 | CD32 |
| CD83 | CD4 | FcγRI-γ | CD83 | OX40 | CD79a |
| CD83 | CD4 | FeγRIII-γ | CD83 | OX40 | CD79b |
| CD83 | CD4 | FcεRIβ | CD83 | DAP10 | CD8 |
| CD83 | CD4 | FcεRIγ | CD83 | DAP10 | CD3ζ |
| CD83 | CD4 | DAP10 | CD83 | DAP10 | CD3δ |
| CD83 | CD4 | DAP12 | CD83 | DAP10 | CD3γ |
| CD83 | CD4 | CD32 | CD83 | DAP10 | CD3ε |
| CD83 | CD4 | CD79a | CD83 | DAP10 | FcγRI-γ |
| CD83 | CD4 | CD79b | CD83 | DAP10 | FcγRIII-γ |
| CD83 | b2c | CD8 | CD83 | DAP10 | FcεRIβ |
| CD83 | b2c | CD3ζ | CD83 | DAP10 | FcεRIγ |
| CD83 | b2c | CD3δ | CD83 | DAP10 | DAP10 |
| CD83 | b2c | CD3γ | CD83 | DAP10 | DAP12 |
| CD83 | b2c | CD3ε | CD83 | DAP10 | CD32 |
| CD83 | b2c | FcγRI-γ | CD83 | DAP10 | CD79a |
| CD83 | b2c | FcγRIII-γ | CD83 | DAP10 | CD79b |
| CD83 | b2c | FcεRIβ | CD83 | DAP12 | CD8 |
| CD83 | b2c | FcεRIγ | CD83 | DAP12 | CD3ζ |
| CD83 | b2c | DAP10 | CD83 | DAP12 | CD3δ |
| CD83 | b2c | DAP12 | CD83 | DAP12 | CD3γ |
| CD83 | b2c | CD32 | CD83 | DAP12 | CD3ε |
| CD83 | b2c | CD79a | CD83 | DAP12 | FcγRI-γ |
| CD83 | b2c | CD79b | CD83 | DAP12 | FcγRIII-γ |
| CD83 | CD137/41BB | CD8 | CD83 | DAP12 | FcεRIβ |
| CD83 | CD137/41BB | CD3ζ | CD83 | DAP12 | FcεRIγ |
| CD83 | CD137/41BB | CD3δ | CD83 | DAP12 | DAP10 |
| CD83 | CD137/41BB | CD3γ | CD83 | DAP12 | DAP12 |
| CD83 | CD137/41BB | CD3ε | CD83 | DAP12 | CD32 |
| CD83 | CD137/41BB | FcγRI-γ | CD83 | DAP12 | CD79a |
| CD83 | CD137/41BB | FcγRIII-γ | CD83 | DAP12 | CD79b |
| CD83 | CD137/41BB | FcεRIβ | CD83 | MyD88 | CD8 |
| CD83 | CD137/41BB | FcεRIγ | CD83 | MyD88 | CD3ζ |
| CD83 | CD137/41BB | DAP10 | CD83 | MyD88 | CD3δ |
| CD83 | CD137/41BB | DAP12 | CD83 | MyD88 | CD3γ |
| CD83 | CD137/41BB | CD32 | CD83 | MyD88 | CD3ε |
| CD83 | CD137/41BB | CD79a | CD83 | MyD88 | FcγRI-γ |
| CD83 | CD137/41BB | CD79b | CD83 | MyD88 | FcγRIII-γ |
| CD83 | ICOS | CD8 | CD83 | MyD88 | FcεRIβ |
| CD83 | ICOS | CD3ζ | CD83 | MyD88 | FcεRIγ |
| CD83 | ICOS | CD3δ | CD83 | MyD88 | DAP10 |
| CD83 | ICOS | CD3γ | CD83 | MyD88 | DAP12 |
| CD83 | ICOS | CD3ε | CD83 | MyD88 | CD32 |
| CD83 | ICOS | FcγRI-γ | CD83 | MyD88 | CD79a |
| CD83 | ICOS | FcγRIII-γ | CD83 | MyD88 | CD79b |
| CD83 | ICOS | FcεRIβ | CD83 | CD7 | CD8 |
| CD83 | ICOS | FcεRIγ | CD83 | CD7 | CD3ζ |
| CD83 | ICOS | DAP10 | CD83 | CD7 | CD3δ |
| CD83 | ICOS | DAP12 | CD83 | CD7 | CD3γ |
| CD83 | ICOS | CD32 | CD83 | CD7 | CD3ε |
| CD83 | ICOS | CD79a | CD83 | CD7 | FcγRI-γ |
| CD83 | ICOS | CD79b | CD83 | CD7 | FcγRIII-γ |
| CD83 | CD27 | CD8 | CD83 | CD7 | FcεRIβ |
| CD83 | CD27 | CD3ζ | CD83 | CD7 | FcεRIγ |
| CD83 | CD27 | CD3δ | CD83 | CD7 | DAP10 |
| CD83 | CD27 | CD3γ | CD83 | CD7 | DAP12 |
| CD83 | CD27 | CD3ε | CD83 | CD7 | CD32 |
| CD83 | CD27 | FcγRI-γ | CD83 | CD7 | CD79a |
| CD83 | CD27 | FcγRIII-γ | CD83 | CD7 | CD79b |
| CD83 | CD27 | FcεRIβ | CD83 | BTNL3 | CD8 |
| CD83 | CD27 | FcεRIγ | CD83 | BTNL3 | CD3ζ |
| CD83 | CD27 | DAP10 | CD83 | BTNL3 | CD3δ |
| CD83 | CD27 | DAP12 | CD83 | BTNL3 | CD3γ |
| CD83 | CD27 | CD32 | CD83 | BTNL3 | CD3ε |
| CD83 | CD27 | CD79a | CD83 | BTNL3 | FcγRI-γ |
| CD83 | CD27 | CD79b | CD83 | BTNL3 | FcγRIII-γ |
| CD83 | CD28δ | CD8 | CD83 | BTNL3 | FcεRIβ |
| CD83 | CD28δ | CD3ζ | CD83 | BTNL3 | FcεRIγ |
| CD83 | CD28δ | CD3δ | CD83 | BTNL3 | DAP10 |
| CD83 | CD28δ | CD3γ | CD83 | BTNL3 | DAP12 |
| CD83 | CD28δ | CD3ε | CD83 | BTNL3 | CD32 |
| CD83 | CD28δ | FcγRI-γ | CD83 | BTNL3 | CD79a |
| CD83 | CD28δ | FcγRIII-γ | CD83 | BTNL3 | CD79b |
| CD83 | CD28δ | FcεRIβ | CD83 | NKG2D | CD8 |
| CD83 | CD28δ | FcεRIγ | CD83 | NKG2D | CD3ζ |
| CD83 | CD28δ | DAP10 | CD83 | NKG2D | CD3δ |
| CD83 | CD28δ | DAP12 | CD83 | NKG2D | CD3γ |
| CD83 | CD28δ | CD32 | CD83 | NKG2D | CD3ε |
| CD83 | CD28δ | CD79a | CD83 | NKG2D | FcγRI-γ |
| CD83 | CD28δ | CD79b | CD83 | NKG2D | FcγRIII-γ |
| CD83 | CD80 | CD8 | CD83 | NKG2D | FcεRIβ |
| CD83 | CD80 | CD3ζ | CD83 | NKG2D | FcεRIγ |
| CD83 | CD80 | CD3δ | CD83 | NKG2D | DAP10 |
| CD83 | CD80 | CD3γ | CD83 | NKG2D | DAP12 |
| CD83 | CD80 | CD3ε | CD83 | NKG2D | CD32 |
| CD83 | CD80 | FcγRI-γ | CD83 | NKG2D | CD79a |
| CD83 | CD80 | FcγRIII-γ | CD83 | NKG2D | CD79b |

TABLE 3

Third Generation CARs

| | | | |
|---|---|---|---|
| ScFv | Co-stimulatory Signal | Co-stimulatory Signal | Signal Domain |
| CD83 | CD28 | CD28 | CD8 |
| CD83 | CD28 | CD28 | CD3ζ |
| CD83 | CD28 | CD28 | CD3δ |
| CD83 | CD28 | CD28 | CD3γ |
| CD83 | CD28 | CD28 | CD3ε |
| CD83 | CD28 | CD28 | FcγRI-γ |
| CD83 | CD28 | CD28 | FcγRIII-γ |
| CD83 | CD28 | CD28 | FcεRIβ |
| CD83 | CD28 | CD28 | FcεRIγ |
| CD83 | CD28 | CD28 | DAP10 |
| CD83 | CD28 | CD28 | DAP12 |
| CD83 | CD28 | CD28 | CD32 |
| CD83 | CD28 | CD28 | CD79a |
| CD83 | CD28 | CD28 | CD79b |
| CD83 | CD28 | CD8 | CD8 |
| CD83 | CD28 | CD8 | CD3ζ |
| CD83 | CD28 | CD8 | CD3δ |
| CD83 | CD28 | CD8 | CD3γ |
| CD83 | CD28 | CD8 | CD3ε |
| CD83 | CD28 | CD8 | FcγRI-γ |
| CD83 | CD28 | CD8 | FcγRIII-γ |
| CD83 | CD28 | CD8 | FcεRIβ |
| CD83 | CD28 | CD8 | FcεRIγ |
| CD83 | CD28 | CD8 | DAP10 |
| CD83 | CD28 | CD8 | DAP12 |
| CD83 | CD28 | CD8 | CD32 |
| CD83 | CD28 | CD8 | CD79a |
| CD83 | CD28 | CD8 | CD79b |
| CD83 | CD28 | CD4 | CD8 |
| CD83 | CD28 | CD4 | CD3ζ |
| CD83 | CD28 | CD4 | CD3δ |
| CD83 | CD28 | CD4 | CD3γ |
| CD83 | CD28 | CD4 | CD3ε |
| CD83 | CD28 | CD4 | FcγRI-γ |
| CD83 | CD28 | CD4 | FcγRIII-γ |
| CD83 | CD28 | CD4 | FcεRIβ |
| CD83 | CD28 | CD4 | FcεRIγ |
| CD83 | CD28 | CD4 | DAP10 |
| CD83 | CD28 | CD4 | DAP12 |
| CD83 | CD28 | CD4 | CD32 |
| CD83 | CD28 | CD4 | CD79a |
| CD83 | CD28 | CD4 | CD79b |
| CD83 | CD28 | b2c | CD8 |
| CD83 | CD28 | b2c | CD3ζ |
| CD83 | CD28 | b2c | CD3δ |
| CD83 | CD28 | b2c | CD3γ |
| CD83 | CD28 | b2c | CD3ε |
| CD83 | CD28 | b2c | FcγRI-γ |
| CD83 | CD28 | b2c | FcγRIII-γ |
| CD83 | CD28 | b2c | FcεRIβ |
| CD83 | CD28 | b2c | FcεRIγ |
| CD83 | CD28 | b2c | DAP10 |
| CD83 | CD28 | b2c | DAP12 |
| CD83 | CD28 | b2c | CD32 |
| CD83 | CD28 | b2c | CD79a |
| CD83 | CD28 | b2c | CD79b |
| CD83 | CD28 | CD137/41BB | CD8 |
| CD83 | CD28 | CD137/41BB | CD3ζ |
| CD83 | CD28 | CD137/41BB | CD3δ |
| CD83 | CD28 | CD137/41BB | CD3γ |
| CD83 | CD28 | CD137/41BB | CD3ε |
| CD83 | CD28 | CD137/41BB | FcγRI-γ |
| CD83 | CD28 | CD137/41BB | FcγRIII-γ |
| CD83 | CD28 | CD137/41BB | FcεRIβ |
| CD83 | CD28 | CD137/41BB | FcεRIγ |
| CD83 | CD28 | CD137/41BB | DAP10 |
| CD83 | CD28 | CD137/41BB | DAP12 |
| CD83 | CD28 | CD137/41BB | CD32 |
| CD83 | CD28 | CD137/41BB | CD79a |
| CD83 | CD28 | CD137/41BB | CD79b |
| CD83 | CD28 | ICOS | CD8 |
| CD83 | CD28 | ICOS | CD3ζ |
| CD83 | CD28 | ICOS | CD3δ |
| CD83 | CD28 | ICOS | CD3γ |
| CD83 | CD28 | ICOS | CD3ε |
| CD83 | CD28 | ICOS | FcγRI-γ |
| CD83 | CD28 | ICOS | FcγRIII-γ |
| CD83 | CD28 | ICOS | FcεRIβ |
| CD83 | CD28 | ICOS | FcεRIγ |
| CD83 | CD28 | ICOS | DAP10 |
| CD83 | CD28 | ICOS | DAP12 |
| CD83 | CD28 | ICOS | CD32 |
| CD83 | CD28 | ICOS | CD79a |
| CD83 | CD28 | ICOS | CD79b |
| CD83 | CD28 | CD27 | CD8 |
| CD83 | CD28 | CD27 | CD3ζ |
| CD83 | CD28 | CD27 | CD3δ |
| CD83 | CD28 | CD27 | CD3γ |
| CD83 | CD28 | CD27 | CD3ε |
| CD83 | CD28 | CD27 | FcγRI-γ |
| CD83 | CD28 | CD27 | FcγRIII-γ |
| CD83 | CD28 | CD27 | FcεRIβ |
| CD83 | CD28 | CD27 | FcεRIγ |
| CD83 | CD28 | CD27 | DAP10 |
| CD83 | CD28 | CD27 | DAP12 |
| CD83 | CD28 | CD27 | CD32 |
| CD83 | CD28 | CD27 | CD79a |
| CD83 | CD28 | CD27 | CD79b |
| CD83 | CD28 | CD28δ | CD8 |
| CD83 | CD28 | CD28δ | CD3ζ |
| CD83 | CD28 | CD28δ | CD3δ |
| CD83 | CD28 | CD28δ | CD3γ |
| CD83 | CD28 | CD28δ | CD3ε |
| CD83 | CD28 | CD28δ | FcγRI-γ |
| CD83 | CD28 | CD28δ | FcγRIII-γ |
| CD83 | CD28 | CD28δ | FcεRIβ |
| CD83 | CD28 | CD28δ | FcεRIγ |
| CD83 | CD28 | CD28δ | DAP10 |
| CD83 | CD28 | CD28δ | DAP12 |
| CD83 | CD28 | CD28δ | CD32 |
| CD83 | CD28 | CD28δ | CD79a |
| CD83 | CD28 | CD28δ | CD79b |
| CD83 | CD28 | CD80 | CD8 |
| CD83 | CD28 | CD80 | CD3ζ |
| CD83 | CD28 | CD80 | CD3δ |
| CD83 | CD28 | CD80 | CD3γ |
| CD83 | CD28 | CD80 | CD3ε |
| CD83 | CD28 | CD80 | FcγRI-γ |
| CD83 | CD28 | CD80 | FcγRIII-γ |
| CD83 | CD28 | CD80 | FcεRIβ |
| CD83 | CD28 | CD80 | FcεRIγ |
| CD83 | CD28 | CD80 | DAP10 |
| CD83 | CD28 | CD80 | DAP12 |
| CD83 | CD28 | CD80 | CD32 |
| CD83 | CD28 | CD80 | CD79a |
| CD83 | CD28 | CD80 | CD79b |
| CD83 | CD28 | CD86 | CD8 |
| CD83 | CD28 | CD86 | CD3ζ |
| CD83 | CD28 | CD86 | CD3δ |
| CD83 | CD28 | CD86 | CD3γ |
| CD83 | CD28 | CD86 | CD3ε |
| CD83 | CD28 | CD86 | FcγRI-γ |
| CD83 | CD28 | CD86 | FcγRIII-γ |
| CD83 | CD28 | CD86 | FcεRIβ |
| CD83 | CD28 | CD86 | FcεRIγ |
| CD83 | CD28 | CD86 | DAP10 |
| CD83 | CD28 | CD86 | DAP12 |
| CD83 | CD28 | CD86 | CD32 |
| CD83 | CD28 | CD86 | CD79a |
| CD83 | CD28 | CD86 | CD79b |
| CD83 | CD28 | OX40 | CD8 |
| CD83 | CD28 | OX40 | CD3ζ |
| CD83 | CD28 | OX40 | CD3δ |
| CD83 | CD28 | OX40 | CD3γ |
| CD83 | CD28 | OX40 | CD3ε |
| CD83 | CD28 | OX40 | FcγRI-γ |
| CD83 | CD28 | OX40 | FcγRIII-γ |
| CD83 | CD28 | OX40 | FcεRIβ |
| CD83 | CD28 | OX40 | FcεRIγ |
| CD83 | CD28 | OX40 | DAP10 |
| CD83 | CD28 | OX40 | DAP12 |
| CD83 | CD28 | OX40 | CD32 |
| CD83 | CD28 | OX40 | CD79a |
| CD83 | CD28 | OX40 | CD79b |
| CD83 | CD28 | DAP10 | CD8 |
| CD83 | CD28 | DAP10 | CD3ζ |
| CD83 | CD28 | DAP10 | CD3δ |
| CD83 | CD28 | DAP10 | CD3γ |
| CD83 | CD28 | DAP10 | CD3ε |
| CD83 | CD28 | DAP10 | FcγRI-γ |
| CD83 | CD28 | DAP10 | FcγRIII-γ |
| CD83 | CD28 | DAP10 | FcεRIβ |
| CD83 | CD28 | DAP10 | FcεRIγ |
| CD83 | CD28 | DAP10 | DAP10 |
| CD83 | CD28 | DAP10 | DAP12 |
| CD83 | CD28 | DAP10 | CD32 |
| CD83 | CD28 | DAP10 | CD79a |
| CD83 | CD28 | DAP10 | CD79b |
| CD83 | CD28 | DAP12 | CD8 |
| CD83 | CD28 | DAP12 | CD3ζ |
| CD83 | CD28 | DAP12 | CD3δ |
| CD83 | CD28 | DAP12 | CD3γ |
| CD83 | CD28 | DAP12 | CD3ε |
| CD83 | CD28 | DAP12 | FcγRI-γ |
| CD83 | CD28 | DAP12 | FcγRIII-γ |
| CD83 | CD28 | DAP12 | FcεRIβ |
| CD83 | CD28 | DAP12 | FcεRIγ |
| CD83 | CD28 | DAP12 | DAP10 |
| CD83 | CD28 | DAP12 | DAP12 |
| CD83 | CD28 | DAP12 | CD32 |
| CD83 | CD28 | DAP12 | CD79a |
| CD83 | CD28 | DAP12 | CD79b |
| CD83 | CD28 | MyD88 | CD8 |
| CD83 | CD28 | MyD88 | CD3ζ |
| CD83 | CD28 | MyD88 | CD3δ |
| CD83 | CD28 | MyD88 | CD3γ |
| CD83 | CD28 | MyD88 | CD3ε |
| CD83 | CD28 | MyD88 | FcγRI-γ |
| CD83 | CD28 | MyD88 | FcγRIII-γ |
| CD83 | CD28 | MyD88 | FcεRIβ |
| CD83 | CD28 | MyD88 | FcεRIγ |
| CD83 | CD28 | MyD88 | DAP10 |
| CD83 | CD28 | MyD88 | DAP12 |
| CD83 | CD28 | MyD88 | CD32 |
| CD83 | CD28 | MyD88 | CD79a |
| CD83 | CD28 | MyD88 | CD79b |
| CD83 | CD28 | CD7 | CD8 |
| CD83 | CD28 | CD7 | CD3ζ |
| CD83 | CD28 | CD7 | CD3δ |
| CD83 | CD28 | CD7 | CD3γ |
| CD83 | CD28 | CD7 | CD3ε |
| CD83 | CD28 | CD7 | FcγRI-γ |
| CD83 | CD28 | CD7 | FcγRIII-γ |
| CD83 | CD28 | CD7 | FcεRIβ |
| CD83 | CD28 | CD7 | FcεRIγ |
| CD83 | CD28 | CD7 | DAP10 |
| CD83 | CD28 | CD7 | DAP12 |
| CD83 | CD28 | CD7 | CD32 |
| CD83 | CD28 | CD7 | CD79a |
| CD83 | CD28 | CD7 | CD79b |
| CD83 | CD28 | BTNL3 | CD8 |
| CD83 | CD28 | BTNL3 | CD3ζ |
| CD83 | CD28 | BTNL3 | CD3δ |
| CD83 | CD28 | BTNL3 | CD3γ |
| CD83 | CD28 | BTNL3 | CD3ε |
| CD83 | CD28 | BTNL3 | FcγRI-γ |
| CD83 | CD28 | BTNL3 | FcγRIII-γ |
| CD83 | CD28 | BTNL3 | FcεRIβ |
| CD83 | CD28 | BTNL3 | FcεRIγ |
| CD83 | CD28 | BTNL3 | DAP10 |
| CD83 | CD28 | BTNL3 | DAP12 |
| CD83 | CD28 | BTNL3 | CD32 |
| CD83 | CD28 | BTNL3 | CD79a |
| CD83 | CD28 | BTNL3 | CD79b |
| CD83 | CD28 | NKG2D | CD8 |
| CD83 | CD28 | NKG2D | CD3ζ |
| CD83 | CD28 | NKG2D | CD3δ |
| CD83 | CD28 | NKG2D | CD3γ |
| CD83 | CD28 | NKG2D | CD3ε |
| CD83 | CD28 | NKG2D | FcγRI-γ |
| CD83 | CD28 | NKG2D | FcγRIII-γ |
| CD83 | CD28 | NKG2D | FcεRIβ |
| CD83 | CD28 | NKG2D | FcεRIγ |
| CD83 | CD28 | NKG2D | DAP10 |
| CD83 | CD28 | NKG2D | DAP12 |
| CD83 | CD28 | NKG2D | CD32 |
| CD83 | CD28 | NKG2D | CD79a |
| CD83 | CD28 | NKG2D | CD79b |
| CD83 | CD8 | CD28 | CD8 |
| CD83 | CD8 | CD28 | CD3ζ |
| CD83 | CD8 | CD28 | CD3δ |
| CD83 | CD8 | CD28 | CD3γ |
| CD83 | CD8 | CD28 | CD3ε |
| CD83 | CD8 | CD28 | FcγRI-γ |
| CD83 | CD8 | CD28 | FcγRIII-γ |
| CD83 | CD8 | CD28 | FcεRIβ |
| CD83 | CD8 | CD28 | FcεRIγ |
| CD83 | CD8 | CD28 | DAP10 |
| CD83 | CD8 | CD28 | DAP12 |
| CD83 | CD8 | CD28 | CD32 |
| CD83 | CD8 | CD28 | CD79a |
| CD83 | CD8 | CD28 | CD79b |
| CD83 | CD8 | CD8 | CD8 |
| CD83 | CD8 | CD8 | CD3ζ |
| CD83 | CD8 | CD8 | CD3δ |
| CD83 | CD8 | CD8 | CD3γ |
| CD83 | CD8 | CD8 | CD3ε |
| CD83 | CD8 | CD8 | FcγRI-γ |
| CD83 | CD8 | CD8 | FcγRIII-γ |
| CD83 | CD8 | CD8 | FcεRIβ |
| CD83 | CD8 | CD8 | FcεRIγ |
| CD83 | CD8 | CD8 | DAP10 |
| CD83 | CD8 | CD8 | DAP12 |
| CD83 | CD8 | CD8 | CD32 |
| CD83 | CD8 | CD8 | CD79a |
| CD83 | CD8 | CD8 | CD79b |
| CD83 | CD8 | CD4 | CD8 |
| CD83 | CD8 | CD4 | CD3ζ |
| CD83 | CD8 | CD4 | CD3δ |
| CD83 | CD8 | CD4 | CD3γ |
| CD83 | CD8 | CD4 | CD3ε |
| CD83 | CD8 | CD4 | FcγRI-γ |
| CD83 | CD8 | CD4 | FcγRIII-γ |
| CD83 | CD8 | CD4 | FcεRIβ |
| CD83 | CD8 | CD4 | FcεRIγ |
| CD83 | CD8 | CD4 | DAP10 |
| CD83 | CD8 | CD4 | DAP12 |
| CD83 | CD8 | CD4 | CD32 |
| CD83 | CD8 | CD4 | CD79a |
| CD83 | CD8 | CD4 | CD79b |
| CD83 | CD8 | b2c | CD8 |
| CD83 | CD8 | b2c | CD3ζ |
| CD83 | CD8 | b2c | CD3δ |
| CD83 | CD8 | b2c | CD3γ |
| CD83 | CD8 | b2c | CD3ε |
| CD83 | CD8 | b2c | FcγRI-γ |
| CD83 | CD8 | b2c | FcγRIII-γ |
| CD83 | CD8 | b2c | FcεRIβ |
| CD83 | CD8 | b2c | FcεRIγ |
| CD83 | CD8 | b2c | DAP10 |
| CD83 | CD8 | b2c | DAP12 |
| CD83 | CD8 | b2c | CD32 |
| CD83 | CD8 | b2c | CD79a |
| CD83 | CD8 | b2c | CD79b |
| CD83 | CD8 | CD137/41BB | CD8 |
| CD83 | CD8 | CD137/41BB | CD3ζ |
| CD83 | CD8 | CD137/41BB | CD3δ |
| CD83 | CD8 | CD137/41BB | CD3γ |
| CD83 | CD8 | CD137/41BB | CD3ε |
| CD83 | CD8 | CD137/41BB | FcγRI-γ |
| CD83 | CD8 | CD137/41BB | FcγRIII-γ |
| CD83 | CD8 | CD137/41BB | FcεRIβ |
| CD83 | CD8 | CD137/41BB | FcεRIγ |
| CD83 | CD8 | CD137/41BB | DAP10 |
| CD83 | CD8 | CD137/41BB | DAP12 |
| CD83 | CD8 | CD137/41BB | CD32 |
| CD83 | CD8 | CD137/41BB | CD79a |
| CD83 | CD8 | CD137/41BB | CD79b |
| CD83 | CD8 | ICOS | CD8 |
| CD83 | CD8 | ICOS | CD3ζ |
| CD83 | CD8 | ICOS | CD3δ |
| CD83 | CD8 | ICOS | CD3γ |
| CD83 | CD8 | ICOS | CD3ε |
| CD83 | CD8 | ICOS | FcγRI-γ |
| CD83 | CD8 | ICOS | FcγRIII-γ |
| CD83 | CD8 | ICOS | FcεRIβ |
| CD83 | CD8 | ICOS | FcεRIγ |
| CD83 | CD8 | ICOS | DAP10 |
| CD83 | CD8 | ICOS | DAP12 |
| CD83 | CD8 | ICOS | CD32 |
| CD83 | CD8 | ICOS | CD79a |
| CD83 | CD8 | ICOS | CD79b |
| CD83 | CD8 | CD27 | CD8 |
| CD83 | CD8 | CD27 | CD3ζ |
| CD83 | CD8 | CD27 | CD3δ |
| CD83 | CD8 | CD27 | CD3γ |
| CD83 | CD8 | CD27 | CD3ε |
| CD83 | CD8 | CD27 | FcγRI-γ |
| CD83 | CD8 | CD27 | FcγRIII-γ |
| CD83 | CD8 | CD27 | FcεRIβ |
| CD83 | CD8 | CD27 | FcεRIγ |
| CD83 | CD8 | CD27 | DAP10 |
| CD83 | CD8 | CD27 | DAP12 |
| CD83 | CD8 | CD27 | CD32 |
| CD83 | CD8 | CD27 | CD79a |
| CD83 | CD8 | CD27 | CD79b |
| CD83 | CD8 | CD28δ | CD8 |
| CD83 | CD8 | CD28δ | CD3ζ |
| CD83 | CD8 | CD28δ | CD3δ |
| CD83 | CD8 | CD28δ | CD3γ |
| CD83 | CD8 | CD28δ | CD3ε |
| CD83 | CD8 | CD28δ | FcγRI-γ |
| CD83 | CD8 | CD28δ | FcγRIII-γ |
| CD83 | CD8 | CD28δ | FcεRIβ |
| CD83 | CD8 | CD28δ | FcεRIγ |
| CD83 | CD8 | CD28δ | DAP10 |
| CD83 | CD8 | CD28δ | DAP12 |
| CD83 | CD8 | CD28δ | CD32 |
| CD83 | CD8 | CD28δ | CD79a |
| CD83 | CD8 | CD28δ | CD79b |
| CD83 | CD8 | CD80 | CD8 |
| CD83 | CD8 | CD80 | CD3ζ |
| CD83 | CD8 | CD80 | CD3δ |
| CD83 | CD8 | CD80 | CD3γ |
| CD83 | CD8 | CD80 | CD3ε |
| CD83 | CD8 | CD80 | FcγRI-γ |
| CD83 | CD8 | CD80 | FcγRIII-γ |
| CD83 | CD8 | CD80 | FcεRIβ |
| CD83 | CD8 | CD80 | FcεRIγ |
| CD83 | CD8 | CD80 | DAP10 |
| CD83 | CD8 | CD80 | DAP12 |
| CD83 | CD8 | CD80 | CD32 |
| CD83 | CD8 | CD80 | CD79a |
| CD83 | CD8 | CD80 | CD79b |
| CD83 | CD8 | CD86 | CD8 |
| CD83 | CD8 | CD86 | CD3ζ |
| CD83 | CD8 | CD86 | CD3δ |
| CD83 | CD8 | CD86 | CD3γ |
| CD83 | CD8 | CD86 | CD3ε |
| CD83 | CD8 | CD86 | FcγRI-γ |
| CD83 | CD8 | CD86 | FcγRIII-γ |
| CD83 | CD8 | CD86 | FcεRIβ |
| CD83 | CD8 | CD86 | FcεRIγ |
| CD83 | CD8 | CD86 | DAP10 |
| CD83 | CD8 | CD86 | DAP12 |
| CD83 | CD8 | CD86 | CD32 |
| CD83 | CD8 | CD86 | CD79a |
| CD83 | CD8 | CD86 | CD79b |
| CD83 | CD8 | OX40 | CD8 |
| CD83 | CD8 | OX40 | CD3ζ |
| CD83 | CD8 | OX40 | CD3δ |
| CD83 | CD8 | OX40 | CD3γ |
| CD83 | CD8 | OX40 | CD3ε |
| CD83 | CD8 | OX40 | FcγRI-γ |
| CD83 | CD8 | OX40 | FcγRIII-γ |
| CD83 | CD8 | OX40 | FcεRIβ |
| CD83 | CD8 | OX40 | FcεRIγ |
| CD83 | CD8 | OX40 | DAP10 |
| CD83 | CD8 | OX40 | DAP12 |
| CD83 | CD8 | OX40 | CD32 |
| CD83 | CD8 | OX40 | CD79a |
| CD83 | CD8 | OX40 | CD79b |
| CD83 | CD8 | DAP10 | CD8 |
| CD83 | CD8 | DAP10 | CD3ζ |
| CD83 | CD8 | DAP10 | CD3δ |
| CD83 | CD8 | DAP10 | CD3γ |
| CD83 | CD8 | DAP10 | CD3ε |
| CD83 | CD8 | DAP10 | FcγRI-γ |
| CD83 | CD8 | DAP10 | FcγRIII-γ |
| CD83 | CD8 | DAP10 | FcεRIβ |
| CD83 | CD8 | DAP10 | FcεRIγ |
| CD83 | CD8 | DAP10 | DAP10 |
| CD83 | CD8 | DAP10 | DAP12 |
| CD83 | CD8 | DAP10 | CD32 |
| CD83 | CD8 | DAP10 | CD79a |
| CD83 | CD8 | DAP10 | CD79b |
| CD83 | CD8 | DAP12 | CD8 |
| CD83 | CD8 | DAP12 | CD3ζ |
| CD83 | CD8 | DAP12 | CD3δ |
| CD83 | CD8 | DAP12 | CD3γ |
| CD83 | CD8 | DAP12 | CD3ε |
| CD83 | CD8 | DAP12 | FcγRI-γ |
| CD83 | CD8 | DAP12 | FcγRIII-γ |
| CD83 | CD8 | DAP12 | FcεRIβ |
| CD83 | CD8 | DAP12 | FcεRIγ |
| CD83 | CD8 | DAP12 | DAP10 |
| CD83 | CD8 | DAP12 | DAP12 |
| CD83 | CD8 | DAP12 | CD32 |
| CD83 | CD8 | DAP12 | CD79a |
| CD83 | CD8 | DAP12 | CD79b |
| CD83 | CD8 | MyD88 | CD8 |
| CD83 | CD8 | MyD88 | CD3ζ |
| CD83 | CD8 | MyD88 | CD3δ |
| CD83 | CD8 | MyD88 | CD3γ |
| CD83 | CD8 | MyD88 | CD3ε |
| CD83 | CD8 | MyD88 | FcγRI-γ |
| CD83 | CD8 | MyD88 | FcγRIII-γ |
| CD83 | CD8 | MyD88 | FcεRIβ |
| CD83 | CD8 | MyD88 | FcεRIγ |
| CD83 | CD8 | MyD88 | DAP10 |
| CD83 | CD8 | MyD88 | DAP12 |
| CD83 | CD8 | MyD88 | CD32 |
| CD83 | CD8 | MyD88 | CD79a |
| CD83 | CD8 | MyD88 | CD79b |
| CD83 | CD8 | CD7 | CD8 |
| CD83 | CD8 | CD7 | CD3ζ |
| CD83 | CD8 | CD7 | CD3δ |
| CD83 | CD8 | CD7 | CD3γ |
| CD83 | CD8 | CD7 | CD3ε |
| CD83 | CD8 | CD7 | FcγRI-γ |
| CD83 | CD8 | CD7 | FcγRIII-γ |
| CD83 | CD8 | CD7 | FcεRIβ |
| CD83 | CD8 | CD7 | FcεRIγ |
| CD83 | CD8 | CD7 | DAP10 |
| CD83 | CD8 | CD7 | DAP12 |
| CD83 | CD8 | CD7 | CD32 |
| CD83 | CD8 | CD7 | CD79a |
| CD83 | CD8 | CD7 | CD79b |
| CD83 | CD8 | BTNL3 | CD8 |
| CD83 | CD8 | BTNL3 | CD3ζ |
| CD83 | CD8 | BTNL3 | CD3δ |
| CD83 | CD8 | BTNL3 | CD3γ |
| CD83 | CD8 | BTNL3 | CD3ε |
| CD83 | CD8 | BTNL3 | FcγRI-γ |
| CD83 | CD8 | BTNL3 | FcγRIII-γ |
| CD83 | CD8 | BTNL3 | FcεRIβ |
| CD83 | CD8 | BTNL3 | FcεRIγ |
| CD83 | CD8 | BTNL3 | DAP10 |
| CD83 | CD8 | BTNL3 | DAP12 |
| CD83 | CD8 | BTNL3 | CD32 |
| CD83 | CD8 | BTNL3 | CD79a |
| CD83 | CD8 | BTNL3 | CD79b |
| CD83 | CD8 | NKG2D | CD8 |
| CD83 | CD8 | NKG2D | CD3ζ |
| CD83 | CD8 | NKG2D | CD3δ |
| CD83 | CD8 | NKG2D | CD3γ |
| CD83 | CD8 | NKG2D | CD3ε |
| CD83 | CD8 | NKG2D | FcγRI-γ |
| CD83 | CD8 | NKG2D | FcγRIII-γ |
| CD83 | CD8 | NKG2D | FcεRIβ |
| CD83 | CD8 | NKG2D | FcεRIγ |
| CD83 | CD8 | NKG2D | DAP10 |
| CD83 | CD8 | NKG2D | DAP12 |
| CD83 | CD8 | NKG2D | CD32 |
| CD83 | CD8 | NKG2D | CD79a |
| CD83 | CD8 | NKG2D | CD79b |
| CD83 | CD4 | CD28 | CD8 |
| CD83 | CD4 | CD28 | CD3ζ |
| CD83 | CD4 | CD28 | CD3δ |
| CD83 | CD4 | CD28 | CD3γ |
| CD83 | CD4 | CD28 | CD3ε |
| CD83 | CD4 | CD28 | FcγRI-γ |
| CD83 | CD4 | CD28 | FcγRIII-γ |
| CD83 | CD4 | CD28 | FcεRIβ |
| CD83 | CD4 | CD28 | FcεRIγ |
| CD83 | CD4 | CD28 | DAP10 |
| CD83 | CD4 | CD28 | DAP12 |
| CD83 | CD4 | CD28 | CD32 |
| CD83 | CD4 | CD28 | CD79a |
| CD83 | CD4 | CD28 | CD79b |
| CD83 | CD4 | CD8 | CD8 |
| CD83 | CD4 | CD8 | CD3ζ |
| CD83 | CD4 | CD8 | CD3δ |
| CD83 | CD4 | CD8 | CD3γ |
| CD83 | CD4 | CD8 | CD3ε |
| CD83 | CD4 | CD8 | FcγRI-γ |
| CD83 | CD4 | CD8 | FcγRIII-γ |
| CD83 | CD4 | CD8 | FcεRIβ |
| CD83 | CD4 | CD8 | FcεRIγ |
| CD83 | CD4 | CD8 | DAP10 |
| CD83 | CD4 | CD8 | DAP12 |
| CD83 | CD4 | CD8 | CD32 |
| CD83 | CD4 | CD8 | CD79a |
| CD83 | CD4 | CD8 | CD79b |
| CD83 | CD4 | CD4 | CD8 |
| CD83 | CD4 | CD4 | CD3ζ |
| CD83 | CD4 | CD4 | CD3δ |
| CD83 | CD4 | CD4 | CD3γ |
| CD83 | CD4 | CD4 | CD3ε |
| CD83 | CD4 | CD4 | FcγRI-γ |
| CD83 | CD4 | CD4 | FcγRIII-γ |
| CD83 | CD4 | CD4 | FcεRIβ |
| CD83 | CD4 | CD4 | FcεRIγ |
| CD83 | CD4 | CD4 | DAP10 |
| CD83 | CD4 | CD4 | DAP12 |
| CD83 | CD4 | CD4 | CD32 |
| CD83 | CD4 | CD4 | CD79a |
| CD83 | CD4 | CD4 | CD79b |
| CD83 | CD4 | b2c | CD8 |
| CD83 | CD4 | b2c | CD3ζ |
| CD83 | CD4 | b2c | CD3δ |
| CD83 | CD4 | b2c | CD3γ |
| CD83 | CD4 | b2c | CD3ε |
| CD83 | CD4 | b2c | FcγRI-γ |
| CD83 | CD4 | b2c | FcγRIII-γ |
| CD83 | CD4 | b2c | FcεRIβ |
| CD83 | CD4 | b2c | FcεRIγ |
| CD83 | CD4 | b2c | DAP10 |
| CD83 | CD4 | b2c | DAP12 |
| CD83 | CD4 | b2c | CD32 |
| CD83 | CD4 | b2c | CD79a |
| CD83 | CD4 | b2c | CD79b |
| CD83 | CD4 | CD137/41BB | CD8 |
| CD83 | CD4 | CD137/41BB | CD3ζ |
| CD83 | CD4 | CD137/41BB | CD3δ |
| CD83 | CD4 | CD137/41BB | CD3γ |
| CD83 | CD4 | CD137/41BB | CD3ε |
| CD83 | CD4 | CD137/41BB | FcγRI-γ |
| CD83 | CD4 | CD137/41BB | FcγRIII-γ |
| CD83 | CD4 | CD137/41BB | FcεRIβ |
| CD83 | CD4 | CD137/41BB | FcεRIγ |
| CD83 | CD4 | CD137/41BB | DAP10 |
| CD83 | CD4 | CD137/41BB | DAP12 |
| CD83 | CD4 | CD137/41BB | CD32 |
| CD83 | CD4 | CD137/41BB | CD79a |
| CD83 | CD4 | CD137/41BB | CD79b |
| CD83 | CD4 | ICOS | CD8 |
| CD83 | CD4 | ICOS | CD3ζ |
| CD83 | CD4 | ICOS | CD3δ |
| CD83 | CD4 | ICOS | CD3γ |
| CD83 | CD4 | ICOS | CD3ε |
| CD83 | CD4 | ICOS | FcγRI-γ |
| CD83 | CD4 | ICOS | FcγRIII-γ |
| CD83 | CD4 | ICOS | FcεRIβ |
| CD83 | CD4 | ICOS | FcεRIγ |
| CD83 | CD4 | ICOS | DAP10 |
| CD83 | CD4 | ICOS | DAP12 |
| CD83 | CD4 | ICOS | CD32 |
| CD83 | CD4 | ICOS | CD79a |
| CD83 | CD4 | ICOS | CD79b |
| CD83 | CD4 | CD27 | CD8 |
| CD83 | CD4 | CD27 | CD3ζ |
| CD83 | CD4 | CD27 | CD3δ |
| CD83 | CD4 | CD27 | CD3γ |
| CD83 | CD4 | CD27 | CD3ε |
| CD83 | CD4 | CD27 | FcγRI-γ |
| CD83 | CD4 | CD27 | FcγRIII-γ |
| CD83 | CD4 | CD27 | FcεRIβ |
| CD83 | CD4 | CD27 | FcεRIγ |
| CD83 | CD4 | CD27 | DAP10 |
| CD83 | CD4 | CD27 | DAP12 |
| CD83 | CD4 | CD27 | CD32 |
| CD83 | CD4 | CD27 | CD79a |
| CD83 | CD4 | CD27 | CD79b |
| CD83 | CD4 | CD28δ | CD8 |
| CD83 | CD4 | CD28δ | CD3ζ |
| CD83 | CD4 | CD28δ | CD3δ |
| CD83 | CD4 | CD28δ | CD3γ |
| CD83 | CD4 | CD28δ | CD3ε |
| CD83 | CD4 | CD28δ | FcγRI-γ |
| CD83 | CD4 | CD28δ | FcγRIII-γ |
| CD83 | CD4 | CD28δ | FcεRIβ |
| CD83 | CD4 | CD28δ | FcεRIγ |
| CD83 | CD4 | CD28δ | DAP10 |
| CD83 | CD4 | CD28δ | DAP12 |
| CD83 | CD4 | CD28δ | CD32 |
| CD83 | CD4 | CD28δ | CD79a |
| CD83 | CD4 | CD28δ | CD79b |
| CD83 | CD4 | CD80 | CD8 |
| CD83 | CD4 | CD80 | CD3ζ |
| CD83 | CD4 | CD80 | CD3δ |
| CD83 | CD4 | CD80 | CD3γ |
| CD83 | CD4 | CD80 | CD3ε |
| CD83 | CD4 | CD80 | FcγRI-γ |
| CD83 | CD4 | CD80 | FcγRIII-γ |
| CD83 | CD4 | CD80 | FcεRIβ |
| CD83 | CD4 | CD80 | FcεRIγ |
| CD83 | CD4 | CD80 | DAP10 |
| CD83 | CD4 | CD80 | DAP12 |
| CD83 | CD4 | CD80 | CD32 |
| CD83 | CD4 | CD80 | CD79a |
| CD83 | CD4 | CD80 | CD79b |
| CD83 | CD4 | CD86 | CD8 |
| CD83 | CD4 | CD86 | CD3ζ |
| CD83 | CD4 | CD86 | CD3δ |
| CD83 | CD4 | CD86 | CD3γ |
| CD83 | CD4 | CD86 | CD3ε |
| CD83 | CD4 | CD86 | FcγRI-γ |
| CD83 | CD4 | CD86 | FcγRIII-γ |
| CD83 | CD4 | CD86 | FcεRIβ |
| CD83 | CD4 | CD86 | FcεRIγ |
| CD83 | CD4 | CD86 | DAP10 |
| CD83 | CD4 | CD86 | DAP12 |
| CD83 | CD4 | CD86 | CD32 |
| CD83 | CD4 | CD86 | CD79a |
| CD83 | CD4 | CD86 | CD79b |
| CD83 | CD4 | OX40 | CD8 |
| CD83 | CD4 | OX40 | CD3ζ |
| CD83 | CD4 | OX40 | CD3δ |
| CD83 | CD4 | OX40 | CD3γ |
| CD83 | CD4 | OX40 | CD3ε |
| CD83 | CD4 | OX40 | FcγRI-γ |
| CD83 | CD4 | OX40 | FcγRIII-γ |
| CD83 | CD4 | OX40 | FcεRIβ |
| CD83 | CD4 | OX40 | FcεRIγ |
| CD83 | CD4 | OX40 | DAP10 |
| CD83 | CD4 | OX40 | DAP12 |
| CD83 | CD4 | OX40 | CD32 |
| CD83 | CD4 | OX40 | CD79a |
| CD83 | CD4 | OX40 | CD79b |
| CD83 | CD4 | DAP10 | CD8 |
| CD83 | CD4 | DAP10 | CD3ζ |
| CD83 | CD4 | DAP10 | CD3δ |
| CD83 | CD4 | DAP10 | CD3γ |
| CD83 | CD4 | DAP10 | CD3ε |
| CD83 | CD4 | DAP10 | FcγRI-γ |
| CD83 | CD4 | DAP10 | FcγRIII-γ |
| CD83 | CD4 | DAP10 | FcεRIβ |
| CD83 | CD4 | DAP10 | FcεRIγ |
| CD83 | CD4 | DAP10 | DAP10 |
| CD83 | CD4 | DAP10 | DAP12 |
| CD83 | CD4 | DAP10 | CD32 |
| CD83 | CD4 | DAP10 | CD79a |
| CD83 | CD4 | DAP10 | CD79b |
| CD83 | CD4 | DAP12 | CD8 |
| CD83 | CD4 | DAP12 | CD3ζ |
| CD83 | CD4 | DAP12 | CD3δ |
| CD83 | CD4 | DAP12 | CD3γ |
| CD83 | CD4 | DAP12 | CD3ε |
| CD83 | CD4 | DAP12 | FcγRI-γ |
| CD83 | CD4 | DAP12 | FcγRIII-γ |
| CD83 | CD4 | DAP12 | FcεRIβ |
| CD83 | CD4 | DAP12 | FcεRIγ |
| CD83 | CD4 | DAP12 | DAP10 |
| CD83 | CD4 | DAP12 | DAP12 |
| CD83 | CD4 | DAP12 | CD32 |
| CD83 | CD4 | DAP12 | CD79a |
| CD83 | CD4 | DAP12 | CD79b |
| CD83 | CD4 | MyD88 | CD8 |
| CD83 | CD4 | MyD88 | CD3ζ |
| CD83 | CD4 | MyD88 | CD3δ |
| CD83 | CD4 | MyD88 | CD3γ |
| CD83 | CD4 | MyD88 | CD3ε |
| CD83 | CD4 | MyD88 | FcγRI-γ |
| CD83 | CD4 | MyD88 | FcγRIII-γ |
| CD83 | CD4 | MyD88 | FcεRIβ |
| CD83 | CD4 | MyD88 | FcεRIγ |
| CD83 | CD4 | MyD88 | DAP10 |
| CD83 | CD4 | MyD88 | DAP12 |
| CD83 | CD4 | MyD88 | CD32 |
| CD83 | CD4 | MyD88 | CD79a |
| CD83 | CD4 | MyD88 | CD79b |
| CD83 | CD4 | CD7 | CD8 |
| CD83 | CD4 | CD7 | CD3ζ |
| CD83 | CD4 | CD7 | CD3δ |
| CD83 | CD4 | CD7 | CD3γ |
| CD83 | CD4 | CD7 | CD3ε |
| CD83 | CD4 | CD7 | FcγRI-γ |
| CD83 | CD4 | CD7 | FcγRIII-γ |
| CD83 | CD4 | CD7 | FcεRIβ |
| CD83 | CD4 | CD7 | FcεRIγ |
| CD83 | CD4 | CD7 | DAP10 |
| CD83 | CD4 | CD7 | DAP12 |
| CD83 | CD4 | CD7 | CD32 |
| CD83 | CD4 | CD7 | CD79a |
| CD83 | CD4 | CD7 | CD79b |
| CD83 | CD4 | BTNL3 | CD8 |
| CD83 | CD4 | BTNL3 | CD3ζ |
| CD83 | CD4 | BTNL3 | CD3δ |
| CD83 | CD4 | BTNL3 | CD3γ |
| CD83 | CD4 | BTNL3 | CD3ε |
| CD83 | CD4 | BTNL3 | FcγRI-γ |
| CD83 | CD4 | BTNL3 | FcγRIII-γ |
| CD83 | CD4 | BTNL3 | FcεRIβ |
| CD83 | CD4 | BTNL3 | FcεRIγ |
| CD83 | CD4 | BTNL3 | DAP10 |
| CD83 | CD4 | BTNL3 | DAP12 |
| CD83 | CD4 | BTNL3 | CD32 |
| CD83 | CD4 | BTNL3 | CD79a |
| CD83 | CD4 | BTNL3 | CD79b |
| CD83 | CD4 | NKG2D | CD8 |
| CD83 | CD4 | NKG2D | CD3ζ |
| CD83 | CD4 | NKG2D | CD3δ |
| CD83 | CD4 | NKG2D | CD3γ |
| CD83 | CD4 | NKG2D | CD3ε |
| CD83 | CD4 | NKG2D | FcγRI-γ |
| CD83 | CD4 | NKG2D | FcγRIII-γ |
| CD83 | CD4 | NKG2D | FcεRIβ |
| CD83 | CD4 | NKG2D | FcεRIγ |
| CD83 | CD4 | NKG2D | DAP10 |
| CD83 | CD4 | NKG2D | DAP12 |
| CD83 | CD4 | NKG2D | CD32 |
| CD83 | CD4 | NKG2D | CD79a |
| CD83 | CD4 | NKG2D | CD79b |
| CD83 | b2c | CD28 | CD8 |
| CD83 | b2c | CD28 | CD3ζ |
| CD83 | b2c | CD28 | CD3δ |
| CD83 | b2c | CD28 | CD3γ |
| CD83 | b2c | CD28 | CD3ε |
| CD83 | b2c | CD28 | FcγRI-γ |
| CD83 | b2c | CD28 | FcγRIII-γ |
| CD83 | b2c | CD28 | FcεRIβ |
| CD83 | b2c | CD28 | FcεRIγ |
| CD83 | b2c | CD28 | DAP10 |
| CD83 | b2c | CD28 | DAP12 |
| CD83 | b2c | CD28 | CD32 |
| CD83 | b2c | CD28 | CD79a |
| CD83 | b2c | CD28 | CD79b |
| CD83 | b2c | CD8 | CD8 |
| CD83 | b2c | CD8 | CD3ζ |
| CD83 | b2c | CD8 | CD3δ |
| CD83 | b2c | CD8 | CD3γ |
| CD83 | b2c | CD8 | CD3ε |
| CD83 | b2c | CD8 | FcγRI-γ |
| CD83 | b2c | CD8 | FcγRIII-γ |
| CD83 | b2c | CD8 | FcεRIβ |
| CD83 | b2c | CD8 | FcεRIγ |
| CD83 | b2c | CD8 | DAP10 |
| CD83 | b2c | CD8 | DAP12 |
| CD83 | b2c | CD8 | CD32 |
| CD83 | b2c | CD8 | CD79a |
| CD83 | b2c | CD8 | CD79b |
| CD83 | b2c | CD4 | CD8 |
| CD83 | b2c | CD4 | CD3ζ |
| CD83 | b2c | CD4 | CD3δ |
| CD83 | b2c | CD4 | CD3γ |
| CD83 | b2c | CD4 | CD3ε |
| CD83 | b2c | CD4 | FcγRI-γ |
| CD83 | b2c | CD4 | FcγRIII-γ |
| CD83 | b2c | CD4 | FcεRIβ |
| CD83 | b2c | CD4 | FcεRIγ |
| CD83 | b2c | CD4 | DAP10 |
| CD83 | b2c | CD4 | DAP12 |
| CD83 | b2c | CD4 | CD32 |
| CD83 | b2c | CD4 | CD79a |
| CD83 | b2c | CD4 | CD79b |
| CD83 | b2c | b2c | CD8 |
| CD83 | b2c | b2c | CD3ζ |
| CD83 | b2c | b2c | CD3δ |
| CD83 | b2c | b2c | CD3γ |
| CD83 | b2c | b2c | CD3ε |
| CD83 | b2c | b2c | FcγRI-γ |
| CD83 | b2c | b2c | FcγRIII-γ |
| CD83 | b2c | b2c | FcεRIβ |
| CD83 | b2c | b2c | FcεRIγ |
| CD83 | b2c | b2c | DAP10 |
| CD83 | b2c | b2c | DAP12 |
| CD83 | b2c | b2c | CD32 |
| CD83 | b2c | b2c | CD79a |
| CD83 | b2c | b2c | CD79b |
| CD83 | b2c | CD137/41BB | CD8 |
| CD83 | b2c | CD137/41BB | CD3ζ |
| CD83 | b2c | CD137/41BB | CD3δ |
| CD83 | b2c | CD137/41BB | CD3γ |
| CD83 | b2c | CD137/41BB | CD3ε |
| CD83 | b2c | CD137/41BB | FcγRI-γ |
| CD83 | b2c | CD137/41BB | FcγRIII-γ |
| CD83 | b2c | CD137/41BB | FcεRIβ |
| CD83 | b2c | CD137/41BB | FcεRIγ |
| CD83 | b2c | CD137/41BB | DAP10 |
| CD83 | b2c | CD137/41BB | DAP12 |
| CD83 | b2c | CD137/41BB | CD32 |
| CD83 | b2c | CD137/41BB | CD79a |
| CD83 | b2c | CD137/41BB | CD79b |
| CD83 | b2c | ICOS | CD8 |
| CD83 | b2c | ICOS | CD3ζ |
| CD83 | b2c | ICOS | CD3δ |
| CD83 | b2c | ICOS | CD3γ |
| CD83 | b2c | ICOS | CD3ε |
| CD83 | b2c | ICOS | FcγRI-γ |
| CD83 | b2c | ICOS | FcγRIII-γ |
| CD83 | b2c | ICOS | FcεRIβ |
| CD83 | b2c | ICOS | FcεRIγ |
| CD83 | b2c | ICOS | DAP10 |
| CD83 | b2c | ICOS | DAP12 |
| CD83 | b2c | ICOS | CD32 |
| CD83 | b2c | ICOS | CD79a |
| CD83 | b2c | ICOS | CD79b |
| CD83 | b2c | CD27 | CD8 |
| CD83 | b2c | CD27 | CD3ζ |
| CD83 | b2c | CD27 | CD3δ |
| CD83 | b2c | CD27 | CD3γ |
| CD83 | b2c | CD27 | CD3ε |
| CD83 | b2c | CD27 | FcγRI-γ |
| CD83 | b2c | CD27 | FcγRIII-γ |
| CD83 | b2c | CD27 | FcεRIβ |
| CD83 | b2c | CD27 | FcεRIγ |
| CD83 | b2c | CD27 | DAP10 |
| CD83 | b2c | CD27 | DAP12 |
| CD83 | b2c | CD27 | CD32 |
| CD83 | b2c | CD27 | CD79a |
| CD83 | b2c | CD27 | CD79b |
| CD83 | b2c | CD28δ | CD8 |
| CD83 | b2c | CD28δ | CD3ζ |
| CD83 | b2c | CD28δ | CD3δ |
| CD83 | b2c | CD28δ | CD3γ |
| CD83 | b2c | CD28δ | CD3ε |
| CD83 | b2c | CD28δ | FcγRI-γ |
| CD83 | b2c | CD28δ | FcγRIII-γ |
| CD83 | b2c | CD28δ | FcεRIβ |
| CD83 | b2c | CD28δ | FcεRIγ |
| CD83 | b2c | CD28δ | DAP10 |
| CD83 | b2c | CD28δ | DAP12 |
| CD83 | b2c | CD28δ | CD32 |
| CD83 | b2c | CD28δ | CD79a |
| CD83 | b2c | CD28δ | CD79b |
| CD83 | b2c | CD80 | CD8 |
| CD83 | b2c | CD80 | CD3ζ |
| CD83 | b2c | CD80 | CD3δ |
| CD83 | b2c | CD80 | CD3γ |
| CD83 | b2c | CD80 | CD3ε |
| CD83 | b2c | CD80 | FcγRI-γ |
| CD83 | b2c | CD80 | FcγRIII-γ |
| CD83 | b2c | CD80 | FcεRIβ |
| CD83 | b2c | CD80 | FcεRIγ |
| CD83 | b2c | CD80 | DAP10 |
| CD83 | b2c | CD80 | DAP12 |
| CD83 | b2c | CD80 | CD32 |
| CD83 | b2c | CD80 | CD79a |
| CD83 | b2c | CD80 | CD79b |
| CD83 | b2c | CD86 | CD8 |
| CD83 | b2c | CD86 | CD3ζ |
| CD83 | b2c | CD86 | CD3δ |
| CD83 | b2c | CD86 | CD3γ |
| CD83 | b2c | CD86 | CD3ε |
| CD83 | b2c | CD86 | FcγRI-γ |
| CD83 | b2c | CD86 | FcγRIII-γ |
| CD83 | b2c | CD86 | FcεRIβ |
| CD83 | b2c | CD86 | FcεRIγ |
| CD83 | b2c | CD86 | DAP10 |
| CD83 | b2c | CD86 | DAP12 |
| CD83 | b2c | CD86 | CD32 |
| CD83 | b2c | CD86 | CD79a |
| CD83 | b2c | CD86 | CD79b |
| CD83 | b2c | OX40 | CD8 |
| CD83 | b2c | OX40 | CD3ζ |
| CD83 | b2c | OX40 | CD3δ |
| CD83 | b2c | OX40 | CD3γ |
| CD83 | b2c | OX40 | CD3ε |
| CD83 | b2c | OX40 | FcγRI-γ |
| CD83 | b2c | OX40 | FcγRIII-γ |
| CD83 | b2c | OX40 | FcεRIβ |
| CD83 | b2c | OX40 | FcεRIγ |
| CD83 | b2c | OX40 | DAP10 |
| CD83 | b2c | OX40 | DAP12 |
| CD83 | b2c | OX40 | CD32 |
| CD83 | b2c | OX40 | CD79a |
| CD83 | b2c | OX40 | CD79b |
| CD83 | b2c | DAP10 | CD8 |
| CD83 | b2c | DAP10 | CD3ζ |
| CD83 | b2c | DAP10 | CD3δ |
| CD83 | b2c | DAP10 | CD3γ |
| CD83 | b2c | DAP10 | CD3ε |
| CD83 | b2c | DAP10 | FcγRI-γ |
| CD83 | b2c | DAP10 | FcγRIII-γ |
| CD83 | b2c | DAP10 | FcεRIβ |
| CD83 | b2c | DAP10 | FcεRIγ |
| CD83 | b2c | DAP10 | DAP10 |
| CD83 | b2c | DAP10 | DAP12 |
| CD83 | b2c | DAP10 | CD32 |
| CD83 | b2c | DAP10 | CD79a |
| CD83 | b2c | DAP10 | CD79b |
| CD83 | b2c | DAP12 | CD8 |
| CD83 | b2c | DAP12 | CD3ζ |
| CD83 | b2c | DAP12 | CD3δ |
| CD83 | b2c | DAP12 | CD3γ |
| CD83 | b2c | DAP12 | CD3ε |
| CD83 | b2c | DAP12 | FcγRI-γ |
| CD83 | b2c | DAP12 | FcγRIII-γ |
| CD83 | b2c | DAP12 | FcεRIβ |
| CD83 | b2c | DAP12 | FcεRIγ |
| CD83 | b2c | DAP12 | DAP10 |
| CD83 | b2c | DAP12 | DAP12 |
| CD83 | b2c | DAP12 | CD32 |
| CD83 | b2c | DAP12 | CD79a |
| CD83 | b2c | DAP12 | CD79b |
| CD83 | b2c | MyD88 | CD8 |
| CD83 | b2c | MyD88 | CD3ζ |
| CD83 | b2c | MyD88 | CD3δ |
| CD83 | b2c | MyD88 | CD3γ |
| CD83 | b2c | MyD88 | CD3ε |
| CD83 | b2c | MyD88 | FcγRI-γ |
| CD83 | b2c | MyD88 | FcγRIII-γ |
| CD83 | b2c | MyD88 | FcεRIβ |
| CD83 | b2c | MyD88 | FcεRIγ |
| CD83 | b2c | MyD88 | DAP10 |
| CD83 | b2c | MyD88 | DAP12 |
| CD83 | b2c | MyD88 | CD32 |
| CD83 | b2c | MyD88 | CD79a |
| CD83 | b2c | MyD88 | CD79b |
| CD83 | b2c | CD7 | CD8 |
| CD83 | b2c | CD7 | CD3ζ |
| CD83 | b2c | CD7 | CD3δ |
| CD83 | b2c | CD7 | CD3γ |
| CD83 | b2c | CD7 | CD3ε |
| CD83 | b2c | CD7 | FcγRI-γ |
| CD83 | b2c | CD7 | FcγRIII-γ |
| CD83 | b2c | CD7 | FcεRIβ |
| CD83 | b2c | CD7 | FcεRIγ |
| CD83 | b2c | CD7 | DAP10 |
| CD83 | b2c | CD7 | DAP12 |
| CD83 | b2c | CD7 | CD32 |
| CD83 | b2c | CD7 | CD79a |
| CD83 | b2c | CD7 | CD79b |
| CD83 | b2c | BTNL3 | CD8 |
| CD83 | b2c | BTNL3 | CD3ζ |
| CD83 | b2c | BTNL3 | CD3δ |
| CD83 | b2c | BTNL3 | CD3γ |
| CD83 | b2c | BTNL3 | CD3ε |
| CD83 | b2c | BTNL3 | FcγRI-γ |
| CD83 | b2c | BTNL3 | FcγRIII-γ |
| CD83 | b2c | BTNL3 | FcεRIβ |
| CD83 | b2c | BTNL3 | FcεRIγ |
| CD83 | b2c | BTNL3 | DAP10 |
| CD83 | b2c | BTNL3 | DAP12 |
| CD83 | b2c | BTNL3 | CD32 |
| CD83 | b2c | BTNL3 | CD79a |
| CD83 | b2c | BTNL3 | CD79b |
| CD83 | b2c | NKG2D | CD8 |
| CD83 | b2c | NKG2D | CD3ζ |
| CD83 | b2c | NKG2D | CD3δ |
| CD83 | b2c | NKG2D | CD3γ |
| CD83 | b2c | NKG2D | CD3ε |
| CD83 | b2c | NKG2D | FcγRI-γ |
| CD83 | b2c | NKG2D | FcγRIII-γ |
| CD83 | b2c | NKG2D | FcεRIβ |
| CD83 | b2c | NKG2D | FcεRIγ |
| CD83 | b2c | NKG2D | DAP10 |
| CD83 | b2c | NKG2D | DAP12 |
| CD83 | b2c | NKG2D | CD32 |
| CD83 | b2c | NKG2D | CD79a |
| CD83 | b2c | NKG2D | CD79b |
| CD83 | CD137/41BB | CD28 | CD8 |
| CD83 | CD137/41BB | CD28 | CD3ζ |
| CD83 | CD137/41BB | CD28 | CD3δ |
| CD83 | CD137/41BB | CD28 | CD3γ |
| CD83 | CD137/41BB | CD28 | CD3ε |
| CD83 | CD137/41BB | CD28 | FcγRI-γ |
| CD83 | CD137/41BB | CD28 | FcγRIII-γ |
| CD83 | CD137/41BB | CD28 | FcεRIβ |
| CD83 | CD137/41BB | CD28 | FcεRIγ |
| CD83 | CD137/41BB | CD28 | DAP10 |
| CD83 | CD137/41BB | CD28 | DAP12 |
| CD83 | CD137/41BB | CD28 | CD32 |
| CD83 | CD137/41BB | CD28 | CD79a |
| CD83 | CD137/41BB | CD28 | CD79b |
| CD83 | CD137/41BB | CD8 | CD8 |
| CD83 | CD137/41BB | CD8 | CD3ζ |
| CD83 | CD137/41BB | CD8 | CD3δ |
| CD83 | CD137/41BB | CD8 | CD3γ |
| CD83 | CD137/41BB | CD8 | CD3ε |
| CD83 | CD137/41BB | CD8 | FcγRI-γ |
| CD83 | CD137/41BB | CD8 | FcγRIII-γ |
| CD83 | CD137/41BB | CD8 | FcεRIβ |
| CD83 | CD137/41BB | CD8 | FcεRIγ |
| CD83 | CD137/41BB | CD8 | DAP10 |
| CD83 | CD137/41BB | CD8 | DAP12 |
| CD83 | CD137/41BB | CD8 | CD32 |
| CD83 | CD137/41BB | CD8 | CD79a |
| CD83 | CD137/41BB | CD8 | CD79b |
| CD83 | CD137/41BB | CD4 | CD8 |
| CD83 | CD137/41BB | CD4 | CD3ζ |
| CD83 | CD137/41BB | CD4 | CD3δ |
| CD83 | CD137/41BB | CD4 | CD3γ |
| CD83 | CD137/41BB | CD4 | CD3ε |
| CD83 | CD137/41BB | CD4 | FcγRI-γ |
| CD83 | CD137/41BB | CD4 | FcγRIII-γ |
| CD83 | CD137/41BB | CD4 | FcεRIβ |
| CD83 | CD137/41BB | CD4 | FcεRIγ |
| CD83 | CD137/41BB | CD4 | DAP10 |
| CD83 | CD137/41BB | CD4 | DAP12 |
| CD83 | CD137/41BB | CD4 | CD32 |
| CD83 | CD137/41BB | CD4 | CD79a |
| CD83 | CD137/41BB | CD4 | CD79b |
| CD83 | CD137/41BB | b2c | CD8 |
| CD83 | CD137/41BB | b2c | CD3ζ |
| CD83 | CD137/41BB | b2c | CD3δ |
| CD83 | CD137/41BB | b2c | CD3γ |
| CD83 | CD137/41BB | b2c | CD3ε |
| CD83 | CD137/41BB | b2c | FcγRI-γ |
| CD83 | CD137/41BB | b2c | FcγRIII-γ |
| CD83 | CD137/41BB | b2c | FcεRIβ |
| CD83 | CD137/41BB | b2c | FcεRIγ |
| CD83 | CD137/41BB | b2c | DAP10 |
| CD83 | CD137/41BB | b2c | DAP12 |
| CD83 | CD137/41BB | b2c | CD32 |
| CD83 | CD137/41BB | b2c | CD79a |
| CD83 | CD137/41BB | b2c | CD79b |
| CD83 | CD137/41BB | CD137/41BB | CD8 |
| CD83 | CD137/41BB | CD137/41BB | CD3ζ |
| CD83 | CD137/41BB | CD137/41BB | CD3δ |
| CD83 | CD137/41BB | CD137/41BB | CD3γ |
| CD83 | CD137/41BB | CD137/41BB | CD3ε |
| CD83 | CD137/41BB | CD137/41BB | FcγRI-γ |
| CD83 | CD137/41BB | CD137/41BB | FcγRIII-γ |
| CD83 | CD137/41BB | CD137/41BB | FcεRIβ |
| CD83 | CD137/41BB | CD137/41BB | FcεRIγ |
| CD83 | CD137/41BB | CD137/41BB | DAP10 |
| CD83 | CD137/41BB | CD137/41BB | DAP12 |
| CD83 | CD137/41BB | CD137/41BB | CD32 |
| CD83 | CD137/41BB | CD137/41BB | CD79a |
| CD83 | CD137/41BB | CD137/41BB | CD79b |
| CD83 | CD137/41BB | ICOS | CD8 |
| CD83 | CD137/41BB | ICOS | CD3ζ |
| CD83 | CD137/41BB | ICOS | CD3δ |
| CD83 | CD137/41BB | ICOS | CD3γ |
| CD83 | CD137/41BB | ICOS | CD3ε |
| CD83 | CD137/41BB | ICOS | FcγRI-γ |
| CD83 | CD137/41BB | ICOS | FcγRIII-γ |
| CD83 | CD137/41BB | ICOS | FcεRIβ |
| CD83 | CD137/41BB | ICOS | FcεRIγ |
| CD83 | CD137/41BB | ICOS | DAP10 |
| CD83 | CD137/41BB | ICOS | DAP12 |
| CD83 | CD137/41BB | ICOS | CD32 |
| CD83 | CD137/41BB | ICOS | CD79a |
| CD83 | CD137/41BB | ICOS | CD79b |
| CD83 | CD137/41BB | CD27 | CD8 |
| CD83 | CD137/41BB | CD27 | CD3ζ |
| CD83 | CD137/41BB | CD27 | CD3δ |
| CD83 | CD137/41BB | CD27 | CD3γ |
| CD83 | CD137/41BB | CD27 | CD3ε |
| CD83 | CD137/41BB | CD27 | FcγRI-γ |
| CD83 | CD137/41BB | CD27 | FcγRIII-γ |
| CD83 | CD137/41BB | CD27 | FcεRIβ |
| CD83 | CD137/41BB | CD27 | FcεRIγ |
| CD83 | CD137/41BB | CD27 | DAP10 |
| CD83 | CD137/41BB | CD27 | DAP12 |
| CD83 | CD137/41BB | CD27 | CD32 |
| CD83 | CD137/41BB | CD27 | CD79a |
| CD83 | CD137/41BB | CD27 | CD79b |
| CD83 | CD137/41BB | CD28δ | CD8 |
| CD83 | CD137/41BB | CD28δ | CD3ζ |
| CD83 | CD137/41BB | CD28δ | CD3δ |
| CD83 | CD137/41BB | CD28δ | CD3γ |
| CD83 | CD137/41BB | CD28δ | CD3ε |
| CD83 | CD137/41BB | CD28δ | FcγRI-γ |
| CD83 | CD137/41BB | CD28δ | FcγRIII-γ |
| CD83 | CD137/41BB | CD28δ | FcεRIβ |
| CD83 | CD137/41BB | CD28δ | FcεRIγ |
| CD83 | CD137/41BB | CD28δ | DAP10 |
| CD83 | CD137/41BB | CD28δ | DAP12 |
| CD83 | CD137/41BB | CD28δ | CD32 |
| CD83 | CD137/41BB | CD28δ | CD79a |
| CD83 | CD137/41BB | CD28δ | CD79b |
| CD83 | CD137/41BB | CD80 | CD8 |
| CD83 | CD137/41BB | CD80 | CD3ζ |
| CD83 | CD137/41BB | CD80 | CD3δ |
| CD83 | CD137/41BB | CD80 | CD3γ |
| CD83 | CD137/41BB | CD80 | CD3ε |
| CD83 | CD137/41BB | CD80 | FcγRI-γ |
| CD83 | CD137/41BB | CD80 | FcγRIII-γ |
| CD83 | CD137/41BB | CD80 | FcεRIβ |
| CD83 | CD137/41BB | CD80 | FcεRIγ |
| CD83 | CD137/41BB | CD80 | DAP10 |
| CD83 | CD137/41BB | CD80 | DAP12 |
| CD83 | CD137/41BB | CD80 | CD32 |
| CD83 | CD137/41BB | CD80 | CD79a |
| CD83 | CD137/41BB | CD80 | CD79b |
| CD83 | CD137/41BB | CD86 | CD8 |
| CD83 | CD137/41BB | CD86 | CD3ζ |
| CD83 | CD137/41BB | CD86 | CD3δ |
| CD83 | CD137/41BB | CD86 | CD3γ |
| CD83 | CD137/41BB | CD86 | CD3ε |
| CD83 | CD137/41BB | CD86 | FcγRI-γ |
| CD83 | CD137/41BB | CD86 | FcγRIII-γ |
| CD83 | CD137/41BB | CD86 | FcεRIβ |
| CD83 | CD137/41BB | CD86 | FcεRIγ |
| CD83 | CD137/41BB | CD86 | DAP10 |
| CD83 | CD137/41BB | CD86 | DAP12 |
| CD83 | CD137/41BB | CD86 | CD32 |
| CD83 | CD137/41BB | CD86 | CD79a |
| CD83 | CD137/41BB | CD86 | CD79b |
| CD83 | CD137/41BB | OX40 | CD8 |
| CD83 | CD137/41BB | OX40 | CD3ζ |
| CD83 | CD137/41BB | OX40 | CD3δ |
| CD83 | CD137/41BB | OX40 | CD3γ |
| CD83 | CD137/41BB | OX40 | CD3ε |
| CD83 | CD137/41BB | OX40 | FcγRI-γ |
| CD83 | CD137/41BB | OX40 | FcγRIII-γ |
| CD83 | CD137/41BB | OX40 | FcεRIβ |
| CD83 | CD137/41BB | OX40 | FcεRIγ |
| CD83 | CD137/41BB | OX40 | DAP10 |
| CD83 | CD137/41BB | OX40 | DAP12 |
| CD83 | CD137/41BB | OX40 | CD32 |
| CD83 | CD137/41BB | OX40 | CD79a |
| CD83 | CD137/41BB | OX40 | CD79b |
| CD83 | CD137/41BB | DAP10 | CD8 |
| CD83 | CD137/41BB | DAP10 | CD3ζ |
| CD83 | CD137/41BB | DAP10 | CD3δ |
| CD83 | CD137/41BB | DAP10 | CD3γ |
| CD83 | CD137/41BB | DAP10 | CD3ε |
| CD83 | CD137/41BB | DAP10 | FcγRI-γ |
| CD83 | CD137/41BB | DAP10 | FcγRIII-γ |
| CD83 | CD137/41BB | DAP10 | FcεRIβ |
| CD83 | CD137/41BB | DAP10 | FcεRIγ |
| CD83 | CD137/41BB | DAP10 | DAP10 |
| CD83 | CD137/41BB | DAP10 | DAP12 |
| CD83 | CD137/41BB | DAP10 | CD32 |
| CD83 | CD137/41BB | DAP10 | CD79a |
| CD83 | CD137/41BB | DAP10 | CD79b |
| CD83 | CD137/41BB | DAP12 | CD8 |
| CD83 | CD137/41BB | DAP12 | CD3ζ |
| CD83 | CD137/41BB | DAP12 | CD3δ |
| CD83 | CD137/41BB | DAP12 | CD3γ |
| CD83 | CD137/41BB | DAP12 | CD3ε |
| CD83 | CD137/41BB | DAP12 | FcγRI-γ |
| CD83 | CD137/41BB | DAP12 | FcγRIII-γ |
| CD83 | CD137/41BB | DAP12 | FcεRIβ |
| CD83 | CD137/41BB | DAP12 | FcεRIγ |
| CD83 | CD137/41BB | DAP12 | DAP10 |
| CD83 | CD137/41BB | DAP12 | DAP12 |
| CD83 | CD137/41BB | DAP12 | CD32 |
| CD83 | CD137/41BB | DAP12 | CD79a |
| CD83 | CD137/41BB | DAP12 | CD79b |
| CD83 | CD137/41BB | MyD88 | CD8 |
| CD83 | CD137/41BB | MyD88 | CD3ζ |
| CD83 | CD137/41BB | MyD88 | CD3δ |
| CD83 | CD137/41BB | MyD88 | CD3γ |
| CD83 | CD137/41BB | MyD88 | CD3ε |
| CD83 | CD137/41BB | MyD88 | FcγRI-γ |
| CD83 | CD137/41BB | MyD88 | FcγRIII-γ |
| CD83 | CD137/41BB | MyD88 | FcεRIβ |
| CD83 | CD137/41BB | MyD88 | FcεRIγ |
| CD83 | CD137/41BB | MyD88 | DAP10 |
| CD83 | CD137/41BB | MyD88 | DAP12 |
| CD83 | CD137/41BB | MyD88 | CD32 |
| CD83 | CD137/41BB | MyD88 | CD79a |
| CD83 | CD137/41BB | MyD88 | CD79b |
| CD83 | CD137/41BB | CD7 | CD8 |
| CD83 | CD137/41BB | CD7 | CD3ζ |
| CD83 | CD137/41BB | CD7 | CD3δ |
| CD83 | CD137/41BB | CD7 | CD3γ |
| CD83 | CD137/41BB | CD7 | CD3ε |
| CD83 | CD137/41BB | CD7 | FcγRI-γ |
| CD83 | CD137/41BB | CD7 | FcγRIII-γ |
| CD83 | CD137/41BB | CD7 | FcεRIβ |
| CD83 | CD137/41BB | CD7 | FcεRIγ |
| CD83 | CD137/41BB | CD7 | DAP10 |
| CD83 | CD137/41BB | CD7 | DAP12 |
| CD83 | CD137/41BB | CD7 | CD32 |
| CD83 | CD137/41BB | CD7 | CD79a |
| CD83 | CD137/41BB | CD7 | CD79b |
| CD83 | CD137/41BB | BTNL3 | CD8 |
| CD83 | CD137/41BB | BTNL3 | CD3ζ |
| CD83 | CD137/41BB | BTNL3 | CD3δ |
| CD83 | CD137/41BB | BTNL3 | CD3γ |
| CD83 | CD137/41BB | BTNL3 | CD3ε |
| CD83 | CD137/41BB | BTNL3 | FcγRI-γ |
| CD83 | CD137/41BB | BTNL3 | FcγRIII-γ |
| CD83 | CD137/41BB | BTNL3 | FcεRIβ |
| CD83 | CD137/41BB | BTNL3 | FcεRIγ |
| CD83 | CD137/41BB | BTNL3 | DAP10 |
| CD83 | CD137/41BB | BTNL3 | DAP12 |
| CD83 | CD137/41BB | BTNL3 | CD32 |
| CD83 | CD137/41BB | BTNL3 | CD79a |
| CD83 | CD137/41BB | BTNL3 | CD79b |
| CD83 | CD137/41BB | NKG2D | CD8 |
| CD83 | CD137/41BB | NKG2D | CD3ζ |
| CD83 | CD137/41BB | NKG2D | CD3δ |
| CD83 | CD137/41BB | NKG2D | CD3γ |
| CD83 | CD137/41BB | NKG2D | CD3ε |
| CD83 | CD137/41BB | NKG2D | FcγRI-γ |
| CD83 | CD137/41BB | NKG2D | FcγRIII-γ |
| CD83 | CD137/41BB | NKG2D | FcεRIβ |
| CD83 | CD137/41BB | NKG2D | FcεRIγ |
| CD83 | CD137/41BB | NKG2D | DAP10 |
| CD83 | CD137/41BB | NKG2D | DAP12 |
| CD83 | CD137/41BB | NKG2D | CD32 |
| CD83 | CD137/41BB | NKG2D | CD79a |
| CD83 | CD137/41BB | NKG2D | CD79b |
| CD83 | ICOS | CD28 | CD8 |
| CD83 | ICOS | CD28 | CD3ζ |
| CD83 | ICOS | CD28 | CD3δ |
| CD83 | ICOS | CD28 | CD3γ |
| CD83 | ICOS | CD28 | CD3ε |
| CD83 | ICOS | CD28 | FcγRI-γ |
| CD83 | ICOS | CD28 | FcγRIII-γ |
| CD83 | ICOS | CD28 | FcεRIβ |
| CD83 | ICOS | CD28 | FcεRIγ |
| CD83 | ICOS | CD28 | DAP10 |
| CD83 | ICOS | CD28 | DAP12 |
| CD83 | ICOS | CD28 | CD32 |
| CD83 | ICOS | CD28 | CD79a |
| CD83 | ICOS | CD28 | CD79b |
| CD83 | ICOS | CD8 | CD8 |
| CD83 | ICOS | CD8 | CD3ζ |
| CD83 | ICOS | CD8 | CD3δ |
| CD83 | ICOS | CD8 | CD3γ |
| CD83 | ICOS | CD8 | CD3ε |
| CD83 | ICOS | CD8 | FcγRI-γ |
| CD83 | ICOS | CD8 | FcγRIII-γ |
| CD83 | ICOS | CD8 | FcεRIβ |
| CD83 | ICOS | CD8 | FcεRIγ |
| CD83 | ICOS | CD8 | DAP10 |
| CD83 | ICOS | CD8 | DAP12 |
| CD83 | ICOS | CD8 | CD32 |
| CD83 | ICOS | CD8 | CD79a |
| CD83 | ICOS | CD8 | CD79b |
| CD83 | ICOS | CD4 | CD8 |
| CD83 | ICOS | CD4 | CD3ζ |
| CD83 | ICOS | CD4 | CD3δ |
| CD83 | ICOS | CD4 | CD3γ |
| CD83 | ICOS | CD4 | CD3ε |
| CD83 | ICOS | CD4 | FcγRI-γ |
| CD83 | ICOS | CD4 | FcγRIII-γ |
| CD83 | ICOS | CD4 | FcεRIβ |
| CD83 | ICOS | CD4 | FcεRIγ |
| CD83 | ICOS | CD4 | DAP10 |
| CD83 | ICOS | CD4 | DAP12 |
| CD83 | ICOS | CD4 | CD32 |
| CD83 | ICOS | CD4 | CD79a |
| CD83 | ICOS | CD4 | CD79b |
| CD83 | ICOS | b2c | CD8 |
| CD83 | ICOS | b2c | CD3ζ |
| CD83 | ICOS | b2c | CD3δ |
| CD83 | ICOS | b2c | CD3γ |
| CD83 | ICOS | b2c | CD3ε |
| CD83 | ICOS | b2c | FcγRI-γ |
| CD83 | ICOS | b2c | FcγRIII-γ |
| CD83 | ICOS | b2c | FcεRIβ |
| CD83 | ICOS | b2c | FcεRIγ |
| CD83 | ICOS | b2c | DAP10 |
| CD83 | ICOS | b2c | DAP12 |
| CD83 | ICOS | b2c | CD32 |
| CD83 | ICOS | b2c | CD79a |
| CD83 | ICOS | b2c | CD79b |
| CD83 | ICOS | CD137/41BB | CD8 |
| CD83 | ICOS | CD137/41BB | CD3ζ |
| CD83 | ICOS | CD137/41BB | CD3δ |
| CD83 | ICOS | CD137/41BB | CD3γ |
| CD83 | ICOS | CD137/41BB | CD3ε |
| CD83 | ICOS | CD137/41BB | FcγRI-γ |
| CD83 | ICOS | CD137/41BB | FcγRIII-γ |
| CD83 | ICOS | CD137/41BB | FcεRIβ |
| CD83 | ICOS | CD137/41BB | FcεRIγ |
| CD83 | ICOS | CD137/41BB | DAP10 |
| CD83 | ICOS | CD137/41BB | DAP12 |
| CD83 | ICOS | CD137/41BB | CD32 |
| CD83 | ICOS | CD137/41BB | CD79a |
| CD83 | ICOS | CD137/41BB | CD79b |
| CD83 | ICOS | ICOS | CD8 |
| CD83 | ICOS | ICOS | CD3ζ |
| CD83 | ICOS | ICOS | CD3δ |
| CD83 | ICOS | ICOS | CD3γ |
| CD83 | ICOS | ICOS | CD3ε |
| CD83 | ICOS | ICOS | FcγRI-γ |
| CD83 | ICOS | ICOS | FcγRIII-γ |
| CD83 | ICOS | ICOS | FcεRIβ |
| CD83 | ICOS | ICOS | FcεRIγ |
| CD83 | ICOS | ICOS | DAP10 |
| CD83 | ICOS | ICOS | DAP12 |
| CD83 | ICOS | ICOS | CD32 |
| CD83 | ICOS | ICOS | CD79a |
| CD83 | ICOS | ICOS | CD79b |
| CD83 | ICOS | CD27 | CD8 |
| CD83 | ICOS | CD27 | CD3ζ |
| CD83 | ICOS | CD27 | CD3δ |
| CD83 | ICOS | CD27 | CD3γ |
| CD83 | ICOS | CD27 | CD3ε |
| CD83 | ICOS | CD27 | FcγRI-γ |
| CD83 | ICOS | CD27 | FcγRIII-γ |
| CD83 | ICOS | CD27 | FcεRIβ |
| CD83 | ICOS | CD27 | FcεRIγ |
| CD83 | ICOS | CD27 | DAP10 |
| CD83 | ICOS | CD27 | DAP12 |
| CD83 | ICOS | CD27 | CD32 |
| CD83 | ICOS | CD27 | CD79a |
| CD83 | ICOS | CD27 | CD79b |
| CD83 | ICOS | CD28δ | CD8 |
| CD83 | ICOS | CD28δ | CD3ζ |
| CD83 | ICOS | CD28δ | CD3δ |
| CD83 | ICOS | CD28δ | CD3γ |
| CD83 | ICOS | CD28δ | CD3ε |
| CD83 | ICOS | CD28δ | FcγRI-γ |
| CD83 | ICOS | CD28δ | FcγRIII-γ |
| CD83 | ICOS | CD28δ | FcεRIβ |
| CD83 | ICOS | CD28δ | FcεRIγ |
| CD83 | ICOS | CD28δ | DAP10 |
| CD83 | ICOS | CD28δ | DAP12 |
| CD83 | ICOS | CD28δ | CD32 |
| CD83 | ICOS | CD28δ | CD79a |
| CD83 | ICOS | CD28δ | CD79b |
| CD83 | ICOS | CD80 | CD8 |
| CD83 | ICOS | CD80 | CD3ζ |
| CD83 | ICOS | CD80 | CD3δ |
| CD83 | ICOS | CD80 | CD3γ |
| CD83 | ICOS | CD80 | CD3ε |
| CD83 | ICOS | CD80 | FcγRI-γ |
| CD83 | ICOS | CD80 | FcγRIII-γ |
| CD83 | ICOS | CD80 | FcεRIβ |
| CD83 | ICOS | CD80 | FcεRIγ |
| CD83 | ICOS | CD80 | DAP10 |
| CD83 | ICOS | CD80 | DAP12 |
| CD83 | ICOS | CD80 | CD32 |
| CD83 | ICOS | CD80 | CD79a |
| CD83 | ICOS | CD80 | CD79b |
| CD83 | ICOS | CD86 | CD8 |
| CD83 | ICOS | CD86 | CD3ζ |
| CD83 | ICOS | CD86 | CD3δ |
| CD83 | ICOS | CD86 | CD3γ |
| CD83 | ICOS | CD86 | CD3ε |
| CD83 | ICOS | CD86 | FcγRI-γ |
| CD83 | ICOS | CD86 | FcγRIII-γ |
| CD83 | ICOS | CD86 | FcεRIβ |
| CD83 | ICOS | CD86 | FcεRIγ |
| CD83 | ICOS | CD86 | DAP10 |
| CD83 | ICOS | CD86 | DAP12 |
| CD83 | ICOS | CD86 | CD32 |
| CD83 | ICOS | CD86 | CD79a |
| CD83 | ICOS | CD86 | CD79b |
| CD83 | ICOS | OX40 | CD8 |
| CD83 | ICOS | OX40 | CD3ζ |
| CD83 | ICOS | OX40 | CD3δ |
| CD83 | ICOS | OX40 | CD3γ |
| CD83 | ICOS | OX40 | CD3ε |
| CD83 | ICOS | OX40 | FcγRI-γ |
| CD83 | ICOS | OX40 | FcγRIII-γ |
| CD83 | ICOS | OX40 | FcεRIβ |
| CD83 | ICOS | OX40 | FcεRIγ |
| CD83 | ICOS | OX40 | DAP10 |
| CD83 | ICOS | OX40 | DAP12 |
| CD83 | ICOS | OX40 | CD32 |
| CD83 | ICOS | OX40 | CD79a |
| CD83 | ICOS | OX40 | CD79b |
| CD83 | ICOS | DAP10 | CD8 |
| CD83 | ICOS | DAP10 | CD3ζ |
| CD83 | ICOS | DAP10 | CD3δ |
| CD83 | ICOS | DAP10 | CD3γ |
| CD83 | ICOS | DAP10 | CD3ε |
| CD83 | ICOS | DAP10 | FcγRI-γ |
| CD83 | ICOS | DAP10 | FcγRIII-γ |
| CD83 | ICOS | DAP10 | FcεRIβ |
| CD83 | ICOS | DAP10 | FcεRIγ |
| CD83 | ICOS | DAP10 | DAP10 |
| CD83 | ICOS | DAP10 | DAP12 |
| CD83 | ICOS | DAP10 | CD32 |
| CD83 | ICOS | DAP10 | CD79a |
| CD83 | ICOS | DAP10 | CD79b |
| CD83 | ICOS | DAP12 | CD8 |
| CD83 | ICOS | DAP12 | CD3ζ |
| CD83 | ICOS | DAP12 | CD3δ |
| CD83 | ICOS | DAP12 | CD3γ |
| CD83 | ICOS | DAP12 | CD3ε |
| CD83 | ICOS | DAP12 | FcγRI-γ |
| CD83 | ICOS | DAP12 | FcγRIII-γ |
| CD83 | ICOS | DAP12 | FcεRIβ |
| CD83 | ICOS | DAP12 | FcεRIγ |
| CD83 | ICOS | DAP12 | DAP10 |
| CD83 | ICOS | DAP12 | DAP12 |
| CD83 | ICOS | DAP12 | CD32 |
| CD83 | ICOS | DAP12 | CD79a |
| CD83 | ICOS | DAP12 | CD79b |
| CD83 | ICOS | MyD88 | CD8 |
| CD83 | ICOS | MyD88 | CD3ζ |
| CD83 | ICOS | MyD88 | CD3δ |
| CD83 | ICOS | MyD88 | CD3γ |
| CD83 | ICOS | MyD88 | CD3ε |
| CD83 | ICOS | MyD88 | FcγRI-γ |
| CD83 | ICOS | MyD88 | FcγRIII-γ |
| CD83 | ICOS | MyD88 | FcεRIβ |
| CD83 | ICOS | MyD88 | FcεRIγ |
| CD83 | ICOS | MyD88 | DAP10 |
| CD83 | ICOS | MyD88 | DAP12 |
| CD83 | ICOS | MyD88 | CD32 |
| CD83 | ICOS | MyD88 | CD79a |
| CD83 | ICOS | MyD88 | CD79b |
| CD83 | ICOS | CD7 | CD8 |
| CD83 | ICOS | CD7 | CD3ζ |
| CD83 | ICOS | CD7 | CD3δ |
| CD83 | ICOS | CD7 | CD3γ |
| CD83 | ICOS | CD7 | CD3ε |
| CD83 | ICOS | CD7 | FcγRI-γ |
| CD83 | ICOS | CD7 | FcγRIII-γ |
| CD83 | ICOS | CD7 | FcεRIβ |
| CD83 | ICOS | CD7 | FcεRIγ |
| CD83 | ICOS | CD7 | DAP10 |
| CD83 | ICOS | CD7 | DAP12 |
| CD83 | ICOS | CD7 | CD32 |
| CD83 | ICOS | CD7 | CD79a |
| CD83 | ICOS | CD7 | CD79b |
| CD83 | ICOS | BTNL3 | CD8 |
| CD83 | ICOS | BTNL3 | CD3ζ |
| CD83 | ICOS | BTNL3 | CD3δ |
| CD83 | ICOS | BTNL3 | CD3γ |
| CD83 | ICOS | BTNL3 | CD3ε |
| CD83 | ICOS | BTNL3 | FcγRI-γ |
| CD83 | ICOS | BTNL3 | FcγRIII-γ |
| CD83 | ICOS | BTNL3 | FcεRIβ |
| CD83 | ICOS | BTNL3 | FcεRIγ |
| CD83 | ICOS | BTNL3 | DAP10 |
| CD83 | ICOS | BTNL3 | DAP12 |
| CD83 | ICOS | BTNL3 | CD32 |
| CD83 | ICOS | BTNL3 | CD79a |
| CD83 | ICOS | BTNL3 | CD79b |
| CD83 | ICOS | NKG2D | CD8 |
| CD83 | ICOS | NKG2D | CD3ζ |
| CD83 | ICOS | NKG2D | CD3δ |
| CD83 | ICOS | NKG2D | CD3γ |
| CD83 | ICOS | NKG2D | CD3ε |
| CD83 | ICOS | NKG2D | FcγRI-γ |
| CD83 | ICOS | NKG2D | FcγRIII-γ |
| CD83 | ICOS | NKG2D | FcεRIβ |
| CD83 | ICOS | NKG2D | FcεRIγ |
| CD83 | ICOS | NKG2D | DAP10 |
| CD83 | ICOS | NKG2D | DAP12 |
| CD83 | ICOS | NKG2D | CD32 |
| CD83 | ICOS | NKG2D | CD79a |
| CD83 | ICOS | NKG2D | CD79b |
| CD83 | CD27 | CD28 | CD8 |
| CD83 | CD27 | CD28 | CD3ζ |
| CD83 | CD27 | CD28 | CD3δ |
| CD83 | CD27 | CD28 | CD3γ |
| CD83 | CD27 | CD28 | CD3ε |
| CD83 | CD27 | CD28 | FcγRI-γ |
| CD83 | CD27 | CD28 | FcγRIII-γ |
| CD83 | CD27 | CD28 | FcεRIβ |
| CD83 | CD27 | CD28 | FcεRIγ |
| CD83 | CD27 | CD28 | DAP10 |
| CD83 | CD27 | CD28 | DAP12 |
| CD83 | CD27 | CD28 | CD32 |
| CD83 | CD27 | CD28 | CD79a |
| CD83 | CD27 | CD28 | CD79b |
| CD83 | CD27 | CD8 | CD8 |
| CD83 | CD27 | CD8 | CD3ζ |
| CD83 | CD27 | CD8 | CD3δ |
| CD83 | CD27 | CD8 | CD3γ |
| CD83 | CD27 | CD8 | CD3ε |
| CD83 | CD27 | CD8 | FcγRI-γ |
| CD83 | CD27 | CD8 | FcγRIII-γ |
| CD83 | CD27 | CD8 | FcεRIβ |
| CD83 | CD27 | CD8 | FcεRIγ |
| CD83 | CD27 | CD8 | DAP10 |
| CD83 | CD27 | CD8 | DAP12 |
| CD83 | CD27 | CD8 | CD32 |
| CD83 | CD27 | CD8 | CD79a |
| CD83 | CD27 | CD8 | CD79b |
| CD83 | CD27 | CD4 | CD8 |
| CD83 | CD27 | CD4 | CD3ζ |
| CD83 | CD27 | CD4 | CD3δ |
| CD83 | CD27 | CD4 | CD3γ |
| CD83 | CD27 | CD4 | CD3ε |
| CD83 | CD27 | CD4 | FcγRI-γ |
| CD83 | CD27 | CD4 | FcγRIII-γ |
| CD83 | CD27 | CD4 | FcεRIβ |
| CD83 | CD27 | CD4 | FcεRIγ |
| CD83 | CD27 | CD4 | DAP10 |
| CD83 | CD27 | CD4 | DAP12 |
| CD83 | CD27 | CD4 | CD32 |
| CD83 | CD27 | CD4 | CD79a |
| CD83 | CD27 | CD4 | CD79b |
| CD83 | CD27 | b2c | CD8 |
| CD83 | CD27 | b2c | CD3ζ |
| CD83 | CD27 | b2c | CD3δ |
| CD83 | CD27 | b2c | CD3γ |
| CD83 | CD27 | b2c | CD3ε |
| CD83 | CD27 | b2c | FcγRI-γ |
| CD83 | CD27 | b2c | FcγRIII-γ |
| CD83 | CD27 | b2c | FcεRIβ |
| CD83 | CD27 | b2c | FcεRIγ |
| CD83 | CD27 | b2c | DAP10 |
| CD83 | CD27 | b2c | DAP12 |
| CD83 | CD27 | b2c | CD32 |
| CD83 | CD27 | b2c | CD79a |
| CD83 | CD27 | b2c | CD79b |
| CD83 | CD27 | CD137/41BB | CD8 |
| CD83 | CD27 | CD137/41BB | CD3ζ |
| CD83 | CD27 | CD137/41BB | CD3δ |
| CD83 | CD27 | CD137/41BB | CD3γ |
| CD83 | CD27 | CD137/41BB | CD3ε |
| CD83 | CD27 | CD137/41BB | FcγRI-γ |
| CD83 | CD27 | CD137/41BB | FcγRIII-γ |
| CD83 | CD27 | CD137/41BB | FcεRIβ |
| CD83 | CD27 | CD137/41BB | FcεRIγ |
| CD83 | CD27 | CD137/41BB | DAP10 |
| CD83 | CD27 | CD137/41BB | DAP12 |
| CD83 | CD27 | CD137/41BB | CD32 |
| CD83 | CD27 | CD137/41BB | CD79a |
| CD83 | CD27 | CD137/41BB | CD79b |
| CD83 | CD27 | ICOS | CD8 |
| CD83 | CD27 | ICOS | CD3ζ |
| CD83 | CD27 | ICOS | CD3δ |
| CD83 | CD27 | ICOS | CD3γ |
| CD83 | CD27 | ICOS | CD3ε |
| CD83 | CD27 | ICOS | FcγRI-γ |
| CD83 | CD27 | ICOS | FcγRIII-γ |
| CD83 | CD27 | ICOS | FcεRIβ |
| CD83 | CD27 | ICOS | FcεRIγ |
| CD83 | CD27 | ICOS | DAP10 |
| CD83 | CD27 | ICOS | DAP12 |
| CD83 | CD27 | ICOS | CD32 |
| CD83 | CD27 | ICOS | CD79a |
| CD83 | CD27 | ICOS | CD79b |
| CD83 | CD27 | CD27 | CD8 |
| CD83 | CD27 | CD27 | CD3ζ |
| CD83 | CD27 | CD27 | CD3δ |
| CD83 | CD27 | CD27 | CD3γ |
| CD83 | CD27 | CD27 | CD3ε |
| CD83 | CD27 | CD27 | FcγRI-γ |
| CD83 | CD27 | CD27 | FcγRIII-γ |
| CD83 | CD27 | CD27 | FcεRIβ |
| CD83 | CD27 | CD27 | FcεRIγ |
| CD83 | CD27 | CD27 | DAP10 |
| CD83 | CD27 | CD27 | DAP12 |
| CD83 | CD27 | CD27 | CD32 |
| CD83 | CD27 | CD27 | CD79a |
| CD83 | CD27 | CD27 | CD79b |
| CD83 | CD27 | CD28δ | CD8 |
| CD83 | CD27 | CD28δ | CD3ζ |
| CD83 | CD27 | CD28δ | CD3δ |
| CD83 | CD27 | CD28δ | CD3γ |
| CD83 | CD27 | CD28δ | CD3ε |
| CD83 | CD27 | CD28δ | FcγRI-γ |
| CD83 | CD27 | CD28δ | FcγRIII-γ |
| CD83 | CD27 | CD28δ | FcεRIβ |
| CD83 | CD27 | CD28δ | FcεRIγ |
| CD83 | CD27 | CD28δ | DAP10 |
| CD83 | CD27 | CD28δ | DAP12 |
| CD83 | CD27 | CD28δ | CD32 |
| CD83 | CD27 | CD28δ | CD79a |
| CD83 | CD27 | CD28δ | CD79b |
| CD83 | CD27 | CD80 | CD8 |
| CD83 | CD27 | CD80 | CD3ζ |
| CD83 | CD27 | CD80 | CD3δ |
| CD83 | CD27 | CD80 | CD3γ |
| CD83 | CD27 | CD80 | CD3ε |
| CD83 | CD27 | CD80 | FcγRI-γ |
| CD83 | CD27 | CD80 | FcγRIII-γ |
| CD83 | CD27 | CD80 | FcεRIβ |
| CD83 | CD27 | CD80 | FcεRIγ |
| CD83 | CD27 | CD80 | DAP10 |
| CD83 | CD27 | CD80 | DAP12 |
| CD83 | CD27 | CD80 | CD32 |
| CD83 | CD27 | CD80 | CD79a |
| CD83 | CD27 | CD80 | CD79b |
| CD83 | CD27 | CD86 | CD8 |
| CD83 | CD27 | CD86 | CD3ζ |
| CD83 | CD27 | CD86 | CD3δ |
| CD83 | CD27 | CD86 | CD3γ |
| CD83 | CD27 | CD86 | CD3ε |
| CD83 | CD27 | CD86 | FcγRI-γ |
| CD83 | CD27 | CD86 | FcγRIII-γ |
| CD83 | CD27 | CD86 | FcεRIβ |
| CD83 | CD27 | CD86 | FcεRIγ |
| CD83 | CD27 | CD86 | DAP10 |
| CD83 | CD27 | CD86 | DAP12 |
| CD83 | CD27 | CD86 | CD32 |
| CD83 | CD27 | CD86 | CD79a |
| CD83 | CD27 | CD86 | CD79b |
| CD83 | CD27 | OX40 | CD8 |
| CD83 | CD27 | OX40 | CD3ζ |
| CD83 | CD27 | OX40 | CD3δ |
| CD83 | CD27 | OX40 | CD3γ |
| CD83 | CD27 | OX40 | CD3ε |
| CD83 | CD27 | OX40 | FcγRI-γ |
| CD83 | CD27 | OX40 | FcγRIII-γ |
| CD83 | CD27 | OX40 | FcεRIβ |
| CD83 | CD27 | OX40 | FcεRIγ |
| CD83 | CD27 | OX40 | DAP10 |
| CD83 | CD27 | OX40 | DAP12 |
| CD83 | CD27 | OX40 | CD32 |
| CD83 | CD27 | OX40 | CD79a |
| CD83 | CD27 | OX40 | CD79b |
| CD83 | CD27 | DAP10 | CD8 |
| CD83 | CD27 | DAP10 | CD3ζ |
| CD83 | CD27 | DAP10 | CD3δ |
| CD83 | CD27 | DAP10 | CD3γ |
| CD83 | CD27 | DAP10 | CD3ε |
| CD83 | CD27 | DAP10 | FcγRI-γ |
| CD83 | CD27 | DAP10 | FcγRIII-γ |
| CD83 | CD27 | DAP10 | FcεRIβ |
| CD83 | CD27 | DAP10 | FcεRIγ |
| CD83 | CD27 | DAP10 | DAP10 |
| CD83 | CD27 | DAP10 | DAP12 |
| CD83 | CD27 | DAP10 | CD32 |
| CD83 | CD27 | DAP10 | CD79a |
| CD83 | CD27 | DAP10 | CD79b |
| CD83 | CD27 | DAP12 | CD8 |
| CD83 | CD27 | DAP12 | CD3ζ |
| CD83 | CD27 | DAP12 | CD3δ |
| CD83 | CD27 | DAP12 | CD3γ |
| CD83 | CD27 | DAP12 | CD3ε |
| CD83 | CD27 | DAP12 | FcγRI-γ |
| CD83 | CD27 | DAP12 | FcγRIII-γ |
| CD83 | CD27 | DAP12 | FcεRIβ |
| CD83 | CD27 | DAP12 | FcεRIγ |
| CD83 | CD27 | DAP12 | DAP10 |
| CD83 | CD27 | DAP12 | DAP12 |
| CD83 | CD27 | DAP12 | CD32 |
| CD83 | CD27 | DAP12 | CD79a |
| CD83 | CD27 | DAP12 | CD79b |
| CD83 | CD27 | MyD88 | CD8 |
| CD83 | CD27 | MyD88 | CD3ζ |
| CD83 | CD27 | MyD88 | CD3δ |
| CD83 | CD27 | MyD88 | CD3γ |
| CD83 | CD27 | MyD88 | CD3ε |
| CD83 | CD27 | MyD88 | FcγRI-γ |
| CD83 | CD27 | MyD88 | FcγRIII-γ |
| CD83 | CD27 | MyD88 | FcεRIβ |
| CD83 | CD27 | MyD88 | FcεRIγ |
| CD83 | CD27 | MyD88 | DAP10 |
| CD83 | CD27 | MyD88 | DAP12 |
| CD83 | CD27 | MyD88 | CD32 |
| CD83 | CD27 | MyD88 | CD79a |
| CD83 | CD27 | MyD88 | CD79b |
| CD83 | CD27 | CD7 | CD8 |
| CD83 | CD27 | CD7 | CD3ζ |
| CD83 | CD27 | CD7 | CD3δ |
| CD83 | CD27 | CD7 | CD3γ |
| CD83 | CD27 | CD7 | CD3ε |
| CD83 | CD27 | CD7 | FcγRI-γ |
| CD83 | CD27 | CD7 | FcγRIII-γ |
| CD83 | CD27 | CD7 | FcεRIβ |
| CD83 | CD27 | CD7 | FcεRIγ |
| CD83 | CD27 | CD7 | DAP10 |
| CD83 | CD27 | CD7 | DAP12 |
| CD83 | CD27 | CD7 | CD32 |
| CD83 | CD27 | CD7 | CD79a |
| CD83 | CD27 | CD7 | CD79b |
| CD83 | CD27 | BTNL3 | CD8 |
| CD83 | CD27 | BTNL3 | CD3ζ |
| CD83 | CD27 | BTNL3 | CD3δ |
| CD83 | CD27 | BTNL3 | CD3γ |
| CD83 | CD27 | BTNL3 | CD3ε |
| CD83 | CD27 | BTNL3 | FcγRI-γ |
| CD83 | CD27 | BTNL3 | FcγRIII-γ |
| CD83 | CD27 | BTNL3 | FcεRIβ |
| CD83 | CD27 | BTNL3 | FcεRIγ |
| CD83 | CD27 | BTNL3 | DAP10 |
| CD83 | CD27 | BTNL3 | DAP12 |
| CD83 | CD27 | BTNL3 | CD32 |
| CD83 | CD27 | BTNL3 | CD79a |
| CD83 | CD27 | BTNL3 | CD79b |
| CD83 | CD27 | NKG2D | CD8 |
| CD83 | CD27 | NKG2D | CD3ζ |
| CD83 | CD27 | NKG2D | CD3δ |
| CD83 | CD27 | NKG2D | CD3γ |
| CD83 | CD27 | NKG2D | CD3ε |
| CD83 | CD27 | NKG2D | FcγRI-γ |
| CD83 | CD27 | NKG2D | FcγRIII-γ |
| CD83 | CD27 | NKG2D | FcεRIβ |
| CD83 | CD27 | NKG2D | FcεRIγ |
| CD83 | CD27 | NKG2D | DAP10 |
| CD83 | CD27 | NKG2D | DAP12 |
| CD83 | CD27 | NKG2D | CD32 |
| CD83 | CD27 | NKG2D | CD79a |
| CD83 | CD27 | NKG2D | CD79b |
| CD83 | CD28δ | CD28 | CD8 |
| CD83 | CD28δ | CD28 | CD3ζ |
| CD83 | CD28δ | CD28 | CD3δ |
| CD83 | CD28δ | CD28 | CD3γ |
| CD83 | CD28δ | CD28 | CD3ε |
| CD83 | CD28δ | CD28 | FcγRI-γ |
| CD83 | CD28δ | CD28 | FcγRIII-γ |
| CD83 | CD28δ | CD28 | FcεRIβ |
| CD83 | CD28δ | CD28 | FcεRIγ |
| CD83 | CD28δ | CD28 | DAP10 |
| CD83 | CD28δ | CD28 | DAP12 |
| CD83 | CD28δ | CD28 | CD32 |
| CD83 | CD28δ | CD28 | CD79a |
| CD83 | CD28δ | CD28 | CD79b |
| CD83 | CD28δ | CD8 | CD8 |
| CD83 | CD28δ | CD8 | CD3ζ |
| CD83 | CD28δ | CD8 | CD3δ |
| CD83 | CD28δ | CD8 | CD3γ |
| CD83 | CD28δ | CD8 | CD3ε |
| CD83 | CD28δ | CD8 | FcγRI-γ |
| CD83 | CD28δ | CD8 | FcγRIII-γ |
| CD83 | CD28δ | CD8 | FcεRIβ |
| CD83 | CD28δ | CD8 | FcεRIγ |
| CD83 | CD28δ | CD8 | DAP10 |
| CD83 | CD28δ | CD8 | DAP12 |
| CD83 | CD28δ | CD8 | CD32 |
| CD83 | CD28δ | CD8 | CD79a |
| CD83 | CD28δ | CD8 | CD79b |
| CD83 | CD28δ | CD4 | CD8 |
| CD83 | CD28δ | CD4 | CD3ζ |
| CD83 | CD28δ | CD4 | CD3δ |
| CD83 | CD28δ | CD4 | CD3γ |
| CD83 | CD28δ | CD4 | CD3ε |
| CD83 | CD28δ | CD4 | FcγRI-γ |
| CD83 | CD28δ | CD4 | FcγRIII-γ |
| CD83 | CD28δ | CD4 | FcεRIβ |
| CD83 | CD28δ | CD4 | FcεRIγ |
| CD83 | CD28δ | CD4 | DAP10 |
| CD83 | CD28δ | CD4 | DAP12 |
| CD83 | CD28δ | CD4 | CD32 |
| CD83 | CD28δ | CD4 | CD79a |
| CD83 | CD28δ | CD4 | CD79b |
| CD83 | CD28δ | b2c | CD8 |
| CD83 | CD28δ | b2c | CD3ζ |
| CD83 | CD28δ | b2c | CD3δ |
| CD83 | CD28δ | b2c | CD3γ |
| CD83 | CD28δ | b2c | CD3ε |
| CD83 | CD28δ | b2c | FcγRI-γ |
| CD83 | CD28δ | b2c | FcγRIII-γ |
| CD83 | CD28δ | b2c | FcεRIβ |
| CD83 | CD28δ | b2c | FcεRIγ |
| CD83 | CD28δ | b2c | DAP10 |
| CD83 | CD28δ | b2c | DAP12 |
| CD83 | CD28δ | b2c | CD32 |
| CD83 | CD28δ | b2c | CD79a |
| CD83 | CD28δ | b2c | CD79b |
| CD83 | CD28δ | CD137/41BB | CD8 |
| CD83 | CD28δ | CD137/41BB | CD3ζ |
| CD83 | CD28δ | CD137/41BB | CD3δ |
| CD83 | CD28δ | CD137/41BB | CD3γ |
| CD83 | CD28δ | CD137/41BB | CD3ε |
| CD83 | CD28δ | CD137/41BB | FcγRI-γ |
| CD83 | CD28δ | CD137/41BB | FcγRIII-γ |
| CD83 | CD28δ | CD137/41BB | FcεRIβ |
| CD83 | CD28δ | CD137/41BB | FcεRIγ |
| CD83 | CD28δ | CD137/41BB | DAP10 |
| CD83 | CD28δ | CD137/41BB | DAP12 |
| CD83 | CD28δ | CD137/41BB | CD32 |
| CD83 | CD28δ | CD137/41BB | CD79a |
| CD83 | CD28δ | CD137/41BB | CD79b |
| CD83 | CD28δ | ICOS | CD8 |
| CD83 | CD28δ | ICOS | CD3ζ |
| CD83 | CD28δ | ICOS | CD3δ |
| CD83 | CD28δ | ICOS | CD3γ |
| CD83 | CD28δ | ICOS | CD3ε |
| CD83 | CD28δ | ICOS | FcγRI-γ |
| CD83 | CD28δ | ICOS | FcγRIII-γ |
| CD83 | CD28δ | ICOS | FcεRIβ |
| CD83 | CD28δ | ICOS | FcεRIγ |
| CD83 | CD28δ | ICOS | DAP10 |
| CD83 | CD28δ | ICOS | DAP12 |
| CD83 | CD28δ | ICOS | CD32 |
| CD83 | CD28δ | ICOS | CD79a |
| CD83 | CD28δ | ICOS | CD79b |
| CD83 | CD28δ | CD27 | CD8 |
| CD83 | CD28δ | CD27 | CD3ζ |
| CD83 | CD28δ | CD27 | CD3δ |
| CD83 | CD28δ | CD27 | CD3γ |
| CD83 | CD28δ | CD27 | CD3ε |
| CD83 | CD28δ | CD27 | FcγRI-γ |
| CD83 | CD28δ | CD27 | FcγRIII-γ |
| CD83 | CD28δ | CD27 | FcεRIβ |
| CD83 | CD28δ | CD27 | FcεRIγ |
| CD83 | CD28δ | CD27 | DAP10 |
| CD83 | CD28δ | CD27 | DAP12 |
| CD83 | CD28δ | CD27 | CD32 |
| CD83 | CD28δ | CD27 | CD79a |
| CD83 | CD28δ | CD27 | CD79b |
| CD83 | CD28δ | CD28δ | CD8 |
| CD83 | CD28δ | CD28δ | CD3ζ |
| CD83 | CD28δ | CD28δ | CD3δ |
| CD83 | CD28δ | CD28δ | CD3γ |
| CD83 | CD28δ | CD28δ | CD3ε |
| CD83 | CD28δ | CD28δ | FcγRI-γ |
| CD83 | CD28δ | CD28δ | FcγRIII-γ |
| CD83 | CD28δ | CD28δ | FcεRIβ |
| CD83 | CD28δ | CD28δ | FcεRIγ |
| CD83 | CD28δ | CD28δ | DAP10 |
| CD83 | CD28δ | CD28δ | DAP12 |
| CD83 | CD28δ | CD28δ | CD32 |
| CD83 | CD28δ | CD28δ | CD79a |
| CD83 | CD28δ | CD28δ | CD79b |
| CD83 | CD28δ | CD80 | CD8 |
| CD83 | CD28δ | CD80 | CD3ζ |
| CD83 | CD28δ | CD80 | CD3δ |
| CD83 | CD28δ | CD80 | CD3γ |
| CD83 | CD28δ | CD80 | CD3ε |
| CD83 | CD28δ | CD80 | FcγRI-γ |
| CD83 | CD28δ | CD80 | FcγRIII-γ |
| CD83 | CD28δ | CD80 | FcεRIβ |
| CD83 | CD28δ | CD80 | FcεRIγ |
| CD83 | CD28δ | CD80 | DAP10 |
| CD83 | CD28δ | CD80 | DAP12 |
| CD83 | CD28δ | CD80 | CD32 |
| CD83 | CD28δ | CD80 | CD79a |
| CD83 | CD28δ | CD80 | CD79b |
| CD83 | CD28δ | CD86 | CD8 |
| CD83 | CD28δ | CD86 | CD3ζ |
| CD83 | CD28δ | CD86 | CD3δ |
| CD83 | CD28δ | CD86 | CD3γ |
| CD83 | CD28δ | CD86 | CD3ε |
| CD83 | CD28δ | CD86 | FcγRI-γ |
| CD83 | CD28δ | CD86 | FcγRIII-γ |
| CD83 | CD28δ | CD86 | FcεRIβ |
| CD83 | CD28δ | CD86 | FcεRIγ |
| CD83 | CD28δ | CD86 | DAP10 |
| CD83 | CD28δ | CD86 | DAP12 |
| CD83 | CD28δ | CD86 | CD32 |
| CD83 | CD28δ | CD86 | CD79a |
| CD83 | CD28δ | CD86 | CD79b |
| CD83 | CD28δ | OX40 | CD8 |
| CD83 | CD28δ | OX40 | CD3ζ |
| CD83 | CD28δ | OX40 | CD3δ |
| CD83 | CD28δ | OX40 | CD3γ |
| CD83 | CD28δ | OX40 | CD3ε |
| CD83 | CD28δ | OX40 | FcγRI-γ |
| CD83 | CD28δ | OX40 | FcγRIII-γ |
| CD83 | CD28δ | OX40 | FcεRIβ |
| CD83 | CD28δ | OX40 | FcεRIγ |
| CD83 | CD28δ | OX40 | DAP10 |
| CD83 | CD28δ | OX40 | DAP12 |
| CD83 | CD28δ | OX40 | CD32 |
| CD83 | CD28δ | OX40 | CD79a |
| CD83 | CD28δ | OX40 | CD79b |
| CD83 | CD28δ | DAP10 | CD8 |
| CD83 | CD28δ | DAP10 | CD3ζ |
| CD83 | CD28δ | DAP10 | CD3δ |
| CD83 | CD28δ | DAP10 | CD3γ |
| CD83 | CD28δ | DAP10 | CD3ε |
| CD83 | CD28δ | DAP10 | FcγRI-γ |
| CD83 | CD28δ | DAP10 | FcγRIII-γ |
| CD83 | CD28δ | DAP10 | FcεRIβ |
| CD83 | CD28δ | DAP10 | FcεRIγ |
| CD83 | CD28δ | DAP10 | DAP10 |
| CD83 | CD28δ | DAP10 | DAP12 |
| CD83 | CD28δ | DAP10 | CD32 |
| CD83 | CD28δ | DAP10 | CD79a |
| CD83 | CD28δ | DAP10 | CD79b |
| CD83 | CD28δ | DAP12 | CD8 |
| CD83 | CD28δ | DAP12 | CD3ζ |
| CD83 | CD28δ | DAP12 | CD3δ |
| CD83 | CD28δ | DAP12 | CD3γ |
| CD83 | CD28δ | DAP12 | CD3ε |
| CD83 | CD28δ | DAP12 | FcγRI-γ |
| CD83 | CD28δ | DAP12 | FcγRIII-γ |
| CD83 | CD28δ | DAP12 | FcεRIβ |
| CD83 | CD28δ | DAP12 | FcεRIγ |
| CD83 | CD28δ | DAP12 | DAP10 |
| CD83 | CD28δ | DAP12 | DAP12 |
| CD83 | CD28δ | DAP12 | CD32 |
| CD83 | CD28δ | DAP12 | CD79a |
| CD83 | CD28δ | DAP12 | CD79b |
| CD83 | CD28δ | MyD88 | CD8 |
| CD83 | CD28δ | MyD88 | CD3ζ |
| CD83 | CD28δ | MyD88 | CD3δ |
| CD83 | CD28δ | MyD88 | CD3γ |
| CD83 | CD28δ | MyD88 | CD3ε |
| CD83 | CD28δ | MyD88 | FcγRI-γ |
| CD83 | CD28δ | MyD88 | FcγRIII-γ |
| CD83 | CD28δ | MyD88 | FcεRIβ |
| CD83 | CD28δ | MyD88 | FcεRIγ |
| CD83 | CD28δ | MyD88 | DAP10 |
| CD83 | CD28δ | MyD88 | DAP12 |
| CD83 | CD28δ | MyD88 | CD32 |
| CD83 | CD28δ | MyD88 | CD79a |
| CD83 | CD28δ | MyD88 | CD79b |
| CD83 | CD28δ | CD7 | CD8 |
| CD83 | CD28δ | CD7 | CD3ζ |
| CD83 | CD28δ | CD7 | CD3δ |
| CD83 | CD28δ | CD7 | CD3γ |
| CD83 | CD28δ | CD7 | CD3ε |
| CD83 | CD28δ | CD7 | FcγRI-γ |
| CD83 | CD28δ | CD7 | FcγRIII-γ |
| CD83 | CD28δ | CD7 | FcεRIβ |
| CD83 | CD28δ | CD7 | FcεRIγ |
| CD83 | CD28δ | CD7 | DAP10 |
| CD83 | CD28δ | CD7 | DAP12 |
| CD83 | CD28δ | CD7 | CD32 |
| CD83 | CD28δ | CD7 | CD79a |
| CD83 | CD28δ | CD7 | CD79b |
| CD83 | CD28δ | BTNL3 | CD8 |
| CD83 | CD28δ | BTNL3 | CD3ζ |
| CD83 | CD28δ | BTNL3 | CD3δ |
| CD83 | CD28δ | BTNL3 | CD3γ |
| CD83 | CD28δ | BTNL3 | CD3ε |
| CD83 | CD28δ | BTNL3 | FcγRI-γ |
| CD83 | CD28δ | BTNL3 | FcγRIII-γ |
| CD83 | CD28δ | BTNL3 | FcεRIβ |
| CD83 | CD28δ | BTNL3 | FcεRIγ |
| CD83 | CD28δ | BTNL3 | DAP10 |
| CD83 | CD28δ | BTNL3 | DAP12 |
| CD83 | CD28δ | BTNL3 | CD32 |
| CD83 | CD28δ | BTNL3 | CD79a |
| CD83 | CD28δ | BTNL3 | CD79b |
| CD83 | CD28δ | NKG2D | CD8 |
| CD83 | CD28δ | NKG2D | CD3ζ |
| CD83 | CD28δ | NKG2D | CD3δ |
| CD83 | CD28δ | NKG2D | CD3γ |
| CD83 | CD28δ | NKG2D | CD3ε |
| CD83 | CD28δ | NKG2D | FcγRI-γ |
| CD83 | CD28δ | NKG2D | FcγRIII-γ |
| CD83 | CD28δ | NKG2D | FcεRIβ |
| CD83 | CD28δ | NKG2D | FcεRIγ |
| CD83 | CD28δ | NKG2D | DAP10 |
| CD83 | CD28δ | NKG2D | DAP12 |
| CD83 | CD28δ | NKG2D | CD32 |
| CD83 | CD28δ | NKG2D | CD79a |
| CD83 | CD28δ | NKG2D | CD79b |
| CD83 | CD80 | CD28 | CD8 |
| CD83 | CD80 | CD28 | CD3ζ |
| CD83 | CD80 | CD28 | CD3δ |
| CD83 | CD80 | CD28 | CD3γ |
| CD83 | CD80 | CD28 | CD3ε |
| CD83 | CD80 | CD28 | FcγRI-γ |
| CD83 | CD80 | CD28 | FcγRIII-γ |
| CD83 | CD80 | CD28 | FcεRIβ |
| CD83 | CD80 | CD28 | FcεRIγ |
| CD83 | CD80 | CD28 | DAP10 |
| CD83 | CD80 | CD28 | DAP12 |
| CD83 | CD80 | CD28 | CD32 |
| CD83 | CD80 | CD28 | CD79a |
| CD83 | CD80 | CD28 | CD79b |
| CD83 | CD80 | CD8 | CD8 |
| CD83 | CD80 | CD8 | CD3ζ |
| CD83 | CD80 | CD8 | CD3δ |
| CD83 | CD80 | CD8 | CD3γ |
| CD83 | CD80 | CD8 | CD3ε |
| CD83 | CD80 | CD8 | FcγRI-γ |
| CD83 | CD80 | CD8 | FcγRIII-γ |
| CD83 | CD80 | CD8 | FcεRIβ |
| CD83 | CD80 | CD8 | FcεRIγ |
| CD83 | CD80 | CD8 | DAP10 |
| CD83 | CD80 | CD8 | DAP12 |
| CD83 | CD80 | CD8 | CD32 |
| CD83 | CD80 | CD8 | CD79a |
| CD83 | CD80 | CD8 | CD79b |
| CD83 | CD80 | CD4 | CD8 |
| CD83 | CD80 | CD4 | CD3ζ |
| CD83 | CD80 | CD4 | CD3δ |
| CD83 | CD80 | CD4 | CD3γ |
| CD83 | CD80 | CD4 | CD3ε |
| CD83 | CD80 | CD4 | FcγRI-γ |
| CD83 | CD80 | CD4 | FcγRIII-γ |
| CD83 | CD80 | CD4 | FcεRIβ |
| CD83 | CD80 | CD4 | FcεRIγ |
| CD83 | CD80 | CD4 | DAP10 |
| CD83 | CD80 | CD4 | DAP12 |
| CD83 | CD80 | CD4 | CD32 |
| CD83 | CD80 | CD4 | CD79a |
| CD83 | CD80 | CD4 | CD79b |
| CD83 | CD80 | b2c | CD8 |
| CD83 | CD80 | b2c | CD3ζ |
| CD83 | CD80 | b2c | CD3δ |
| CD83 | CD80 | b2c | CD3γ |
| CD83 | CD80 | b2c | CD3ε |
| CD83 | CD80 | b2c | FcγRI-γ |
| CD83 | CD80 | b2c | FcγRIII-γ |
| CD83 | CD80 | b2c | FcεRIβ |
| CD83 | CD80 | b2c | FcεRIγ |
| CD83 | CD80 | b2c | DAP10 |
| CD83 | CD80 | b2c | DAP12 |
| CD83 | CD80 | b2c | CD32 |
| CD83 | CD80 | b2c | CD79a |
| CD83 | CD80 | b2c | CD79b |
| CD83 | CD80 | CD137/41BB | CD8 |
| CD83 | CD80 | CD137/41BB | CD3ζ |
| CD83 | CD80 | CD137/41BB | CD3δ |
| CD83 | CD80 | CD137/41BB | CD3γ |
| CD83 | CD80 | CD137/41BB | CD3ε |
| CD83 | CD80 | CD137/41BB | FcγRI-γ |
| CD83 | CD80 | CD137/41BB | FcγRIII-γ |
| CD83 | CD80 | CD137/41BB | FcεRIβ |
| CD83 | CD80 | CD137/41BB | FcεRIγ |
| CD83 | CD80 | CD137/41BB | DAP10 |
| CD83 | CD80 | CD137/41BB | DAP12 |
| CD83 | CD80 | CD137/41BB | CD32 |
| CD83 | CD80 | CD137/41BB | CD79a |
| CD83 | CD80 | CD137/41BB | CD79b |
| CD83 | CD80 | ICOS | CD8 |
| CD83 | CD80 | ICOS | CD3ζ |
| CD83 | CD80 | ICOS | CD3δ |
| CD83 | CD80 | ICOS | CD3γ |
| CD83 | CD80 | ICOS | CD3ε |
| CD83 | CD80 | ICOS | FcγRI-γ |
| CD83 | CD80 | ICOS | FcγRIII-γ |
| CD83 | CD80 | ICOS | FcεRIβ |
| CD83 | CD80 | ICOS | FcεRIγ |
| CD83 | CD80 | ICOS | DAP10 |
| CD83 | CD80 | ICOS | DAP12 |
| CD83 | CD80 | ICOS | CD32 |
| CD83 | CD80 | ICOS | CD79a |
| CD83 | CD80 | ICOS | CD79b |
| CD83 | CD80 | CD27 | CD8 |
| CD83 | CD80 | CD27 | CD3ζ |
| CD83 | CD80 | CD27 | CD3δ |
| CD83 | CD80 | CD27 | CD3γ |
| CD83 | CD80 | CD27 | CD3ε |
| CD83 | CD80 | CD27 | FcγRI-γ |
| CD83 | CD80 | CD27 | FcγRIII-γ |
| CD83 | CD80 | CD27 | FcεRIβ |
| CD83 | CD80 | CD27 | FcεRIγ |
| CD83 | CD80 | CD27 | DAP10 |
| CD83 | CD80 | CD27 | DAP12 |
| CD83 | CD80 | CD27 | CD32 |
| CD83 | CD80 | CD27 | CD79a |
| CD83 | CD80 | CD27 | CD79b |
| CD83 | CD80 | CD28δ | CD8 |
| CD83 | CD80 | CD28δ | CD3ζ |
| CD83 | CD80 | CD28δ | CD3δ |
| CD83 | CD80 | CD28δ | CD3γ |
| CD83 | CD80 | CD28δ | CD3ε |
| CD83 | CD80 | CD28δ | FcγRI-γ |
| CD83 | CD80 | CD28δ | FcγRIII-γ |
| CD83 | CD80 | CD28δ | FcεRIβ |
| CD83 | CD80 | CD28δ | FcεRIγ |
| CD83 | CD80 | CD28δ | DAP10 |
| CD83 | CD80 | CD28δ | DAP12 |
| CD83 | CD80 | CD28δ | CD32 |
| CD83 | CD80 | CD28δ | CD79a |
| CD83 | CD80 | CD28δ | CD79b |
| CD83 | CD80 | CD80 | CD8 |
| CD83 | CD80 | CD80 | CD3ζ |
| CD83 | CD80 | CD80 | CD3δ |
| CD83 | CD80 | CD80 | CD3γ |
| CD83 | CD80 | CD80 | CD3ε |
| CD83 | CD80 | CD80 | FcγRI-γ |
| CD83 | CD80 | CD80 | FcγRIII-γ |
| CD83 | CD80 | CD80 | FcεRIβ |
| CD83 | CD80 | CD80 | FcεRIγ |
| CD83 | CD80 | CD80 | DAP10 |
| CD83 | CD80 | CD80 | DAP12 |
| CD83 | CD80 | CD80 | CD32 |
| CD83 | CD80 | CD80 | CD79a |
| CD83 | CD80 | CD80 | CD79b |
| CD83 | CD80 | CD86 | CD8 |
| CD83 | CD80 | CD86 | CD3ζ |
| CD83 | CD80 | CD86 | CD3δ |
| CD83 | CD80 | CD86 | CD3γ |
| CD83 | CD80 | CD86 | CD3ε |
| CD83 | CD80 | CD86 | FcγRI-γ |
| CD83 | CD80 | CD86 | FcγRIII-γ |
| CD83 | CD80 | CD86 | FcεRIβ |
| CD83 | CD80 | CD86 | FcεRIγ |
| CD83 | CD80 | CD86 | DAP10 |
| CD83 | CD80 | CD86 | DAP12 |
| CD83 | CD80 | CD86 | CD32 |
| CD83 | CD80 | CD86 | CD79a |
| CD83 | CD80 | CD86 | CD79b |
| CD83 | CD80 | OX40 | CD8 |
| CD83 | CD80 | OX40 | CD3ζ |
| CD83 | CD80 | OX40 | CD3δ |
| CD83 | CD80 | OX40 | CD3γ |
| CD83 | CD80 | OX40 | CD3ε |
| CD83 | CD80 | OX40 | FcγRI-γ |
| CD83 | CD80 | OX40 | FcγRIII-γ |
| CD83 | CD80 | OX40 | FcεRIβ |
| CD83 | CD80 | OX40 | FcεRIγ |
| CD83 | CD80 | OX40 | DAP10 |
| CD83 | CD80 | OX40 | DAP12 |
| CD83 | CD80 | OX40 | CD32 |
| CD83 | CD80 | OX40 | CD79a |
| CD83 | CD80 | OX40 | CD79b |
| CD83 | CD80 | DAP10 | CD8 |
| CD83 | CD80 | DAP10 | CD3ζ |
| CD83 | CD80 | DAP10 | CD3δ |
| CD83 | CD80 | DAP10 | CD3γ |
| CD83 | CD80 | DAP10 | CD3ε |
| CD83 | CD80 | DAP10 | FcγRI-γ |
| CD83 | CD80 | DAP10 | FcγRIII-γ |
| CD83 | CD80 | DAP10 | FcεRIβ |
| CD83 | CD80 | DAP10 | FcεRIγ |
| CD83 | CD80 | DAP10 | DAP10 |
| CD83 | CD80 | DAP10 | DAP12 |
| CD83 | CD80 | DAP10 | CD32 |
| CD83 | CD80 | DAP10 | CD79a |
| CD83 | CD80 | DAP10 | CD79b |
| CD83 | CD80 | DAP12 | CD8 |
| CD83 | CD80 | DAP12 | CD3ζ |
| CD83 | CD80 | DAP12 | CD3δ |
| CD83 | CD80 | DAP12 | CD3γ |
| CD83 | CD80 | DAP12 | CD3ε |
| CD83 | CD80 | DAP12 | FcγRI-γ |
| CD83 | CD80 | DAP12 | FcγRIII-γ |
| CD83 | CD80 | DAP12 | FcεRIβ |
| CD83 | CD80 | DAP12 | FcεRIγ |
| CD83 | CD80 | DAP12 | DAP10 |
| CD83 | CD80 | DAP12 | DAP12 |
| CD83 | CD80 | DAP12 | CD32 |
| CD83 | CD80 | DAP12 | CD79a |
| CD83 | CD80 | DAP12 | CD79b |
| CD83 | CD80 | MyD88 | CD8 |
| CD83 | CD80 | MyD88 | CD3ζ |
| CD83 | CD80 | MyD88 | CD3δ |
| CD83 | CD80 | MyD88 | CD3γ |
| CD83 | CD80 | MyD88 | CD3ε |
| CD83 | CD80 | MyD88 | FcγRI-γ |
| CD83 | CD80 | MyD88 | FcγRIII-γ |
| CD83 | CD80 | MyD88 | FcεRIβ |
| CD83 | CD80 | MyD88 | FcεRIγ |
| CD83 | CD80 | MyD88 | DAP10 |
| CD83 | CD80 | MyD88 | DAP12 |
| CD83 | CD80 | MyD88 | CD32 |
| CD83 | CD80 | MyD88 | CD79a |
| CD83 | CD80 | MyD88 | CD79b |
| CD83 | CD80 | CD7 | CD8 |
| CD83 | CD80 | CD7 | CD3ζ |
| CD83 | CD80 | CD7 | CD3δ |
| CD83 | CD80 | CD7 | CD3γ |
| CD83 | CD80 | CD7 | CD3ε |
| CD83 | CD80 | CD7 | FcγRI-γ |
| CD83 | CD80 | CD7 | FcγRIII-γ |
| CD83 | CD80 | CD7 | FcεRIβ |
| CD83 | CD80 | CD7 | FcεRIγ |
| CD83 | CD80 | CD7 | DAP10 |
| CD83 | CD80 | CD7 | DAP12 |
| CD83 | CD80 | CD7 | CD32 |
| CD83 | CD80 | CD7 | CD79a |
| CD83 | CD80 | CD7 | CD79b |
| CD83 | CD80 | BTNL3 | CD8 |
| CD83 | CD80 | BTNL3 | CD3ζ |
| CD83 | CD80 | BTNL3 | CD3δ |
| CD83 | CD80 | BTNL3 | CD3γ |
| CD83 | CD80 | BTNL3 | CD3ε |
| CD83 | CD80 | BTNL3 | FcγRI-γ |
| CD83 | CD80 | BTNL3 | FcγRIII-γ |
| CD83 | CD80 | BTNL3 | FcεRIβ |
| CD83 | CD80 | BTNL3 | FcεRIγ |
| CD83 | CD80 | BTNL3 | DAP10 |
| CD83 | CD80 | BTNL3 | DAP12 |
| CD83 | CD80 | BTNL3 | CD32 |
| CD83 | CD80 | BTNL3 | CD79a |
| CD83 | CD80 | BTNL3 | CD79b |
| CD83 | CD80 | NKG2D | CD8 |
| CD83 | CD80 | NKG2D | CD3ζ |
| CD83 | CD80 | NKG2D | CD3δ |
| CD83 | CD80 | NKG2D | CD3γ |
| CD83 | CD80 | NKG2D | CD3ε |
| CD83 | CD80 | NKG2D | FcγRI-γ |
| CD83 | CD80 | NKG2D | FcγRIII-γ |
| CD83 | CD80 | NKG2D | FcεRIβ |
| CD83 | CD80 | NKG2D | FcεRIγ |
| CD83 | CD80 | NKG2D | DAP10 |
| CD83 | CD80 | NKG2D | DAP12 |
| CD83 | CD80 | NKG2D | CD32 |
| CD83 | CD80 | NKG2D | CD79a |
| CD83 | CD80 | NKG2D | CD79b |
| CD83 | CD86 | CD28 | CD8 |
| CD83 | CD86 | CD28 | CD3ζ |
| CD83 | CD86 | CD28 | CD3δ |
| CD83 | CD86 | CD28 | CD3γ |
| CD83 | CD86 | CD28 | CD3ε |
| CD83 | CD86 | CD28 | FcγRI-γ |
| CD83 | CD86 | CD28 | FcγRIII-γ |
| CD83 | CD86 | CD28 | FcεRIβ |
| CD83 | CD86 | CD28 | FcεRIγ |
| CD83 | CD86 | CD28 | DAP10 |
| CD83 | CD86 | CD28 | DAP12 |
| CD83 | CD86 | CD28 | CD32 |
| CD83 | CD86 | CD28 | CD79a |
| CD83 | CD86 | CD28 | CD79b |
| CD83 | CD86 | CD8 | CD8 |
| CD83 | CD86 | CD8 | CD3ζ |
| CD83 | CD86 | CD8 | CD3δ |
| CD83 | CD86 | CD8 | CD3γ |
| CD83 | CD86 | CD8 | CD3ε |
| CD83 | CD86 | CD8 | FcγRI-γ |
| CD83 | CD86 | CD8 | FcγRIII-γ |
| CD83 | CD86 | CD8 | FcεRIβ |
| CD83 | CD86 | CD8 | FcεRIγ |
| CD83 | CD86 | CD8 | DAP10 |
| CD83 | CD86 | CD8 | DAP12 |
| CD83 | CD86 | CD8 | CD32 |
| CD83 | CD86 | CD8 | CD79a |
| CD83 | CD86 | CD8 | CD79b |
| CD83 | CD86 | CD4 | CD8 |
| CD83 | CD86 | CD4 | CD3ζ |
| CD83 | CD86 | CD4 | CD3δ |
| CD83 | CD86 | CD4 | CD3γ |
| CD83 | CD86 | CD4 | CD3ε |
| CD83 | CD86 | CD4 | FcγRI-γ |
| CD83 | CD86 | CD4 | FcγRIII-γ |
| CD83 | CD86 | CD4 | FcεRIβ |
| CD83 | CD86 | CD4 | FcεRIγ |
| CD83 | CD86 | CD4 | DAP10 |
| CD83 | CD86 | CD4 | DAP12 |
| CD83 | CD86 | CD4 | CD32 |
| CD83 | CD86 | CD4 | CD79a |
| CD83 | CD86 | CD4 | CD79b |
| CD83 | CD86 | b2c | CD8 |
| CD83 | CD86 | b2c | CD3ζ |
| CD83 | CD86 | b2c | CD3δ |
| CD83 | CD86 | b2c | CD3γ |
| CD83 | CD86 | b2c | CD3ε |
| CD83 | CD86 | b2c | FcγRI-γ |
| CD83 | CD86 | b2c | FcγRIII-γ |
| CD83 | CD86 | b2c | FcεRIβ |
| CD83 | CD86 | b2c | FcεRIγ |
| CD83 | CD86 | b2c | DAP10 |
| CD83 | CD86 | b2c | DAP12 |
| CD83 | CD86 | b2c | CD32 |
| CD83 | CD86 | b2c | CD79a |
| CD83 | CD86 | b2c | CD79b |
| CD83 | CD86 | CD137/41BB | CD8 |
| CD83 | CD86 | CD137/41BB | CD3ζ |
| CD83 | CD86 | CD137/41BB | CD3δ |
| CD83 | CD86 | CD137/41BB | CD3γ |
| CD83 | CD86 | CD137/41BB | CD3ε |
| CD83 | CD86 | CD137/41BB | FcγRI-γ |
| CD83 | CD86 | CD137/41BB | FcγRIII-γ |
| CD83 | CD86 | CD137/41BB | FcεRIβ |
| CD83 | CD86 | CD137/41BB | FcεRIγ |
| CD83 | CD86 | CD137/41BB | DAP10 |
| CD83 | CD86 | CD137/41BB | DAP12 |
| CD83 | CD86 | CD137/41BB | CD32 |
| CD83 | CD86 | CD137/41BB | CD79a |
| CD83 | CD86 | CD137/41BB | CD79b |
| CD83 | CD86 | ICOS | CD8 |
| CD83 | CD86 | ICOS | CD3ζ |
| CD83 | CD86 | ICOS | CD3δ |
| CD83 | CD86 | ICOS | CD3γ |
| CD83 | CD86 | ICOS | CD3ε |
| CD83 | CD86 | ICOS | FcγRI-γ |
| CD83 | CD86 | ICOS | FcγRIII-γ |
| CD83 | CD86 | ICOS | FcεRIβ |
| CD83 | CD86 | ICOS | FcεRIγ |
| CD83 | CD86 | ICOS | DAP10 |
| CD83 | CD86 | ICOS | DAP12 |
| CD83 | CD86 | ICOS | CD32 |
| CD83 | CD86 | ICOS | CD79a |
| CD83 | CD86 | ICOS | CD79b |
| CD83 | CD86 | CD27 | CD8 |
| CD83 | CD86 | CD27 | CD3ζ |
| CD83 | CD86 | CD27 | CD3δ |
| CD83 | CD86 | CD27 | CD3γ |
| CD83 | CD86 | CD27 | CD3ε |
| CD83 | CD86 | CD27 | FcγRI-γ |
| CD83 | CD86 | CD27 | FcγRIII-γ |
| CD83 | CD86 | CD27 | FcεRIβ |
| CD83 | CD86 | CD27 | FcεRIγ |
| CD83 | CD86 | CD27 | DAP10 |
| CD83 | CD86 | CD27 | DAP12 |
| CD83 | CD86 | CD27 | CD32 |
| CD83 | CD86 | CD27 | CD79a |
| CD83 | CD86 | CD27 | CD79b |
| CD83 | CD86 | CD28δ | CD8 |
| CD83 | CD86 | CD28δ | CD3ζ |
| CD83 | CD86 | CD28δ | CD3δ |
| CD83 | CD86 | CD28δ | CD3γ |
| CD83 | CD86 | CD28δ | CD3ε |
| CD83 | CD86 | CD28δ | FcγRI-γ |
| CD83 | CD86 | CD28δ | FcγRIII-γ |
| CD83 | CD86 | CD28δ | FcεRIβ |
| CD83 | CD86 | CD28δ | FcεRIγ |
| CD83 | CD86 | CD28δ | DAP10 |
| CD83 | CD86 | CD28δ | DAP12 |
| CD83 | CD86 | CD28δ | CD32 |
| CD83 | CD86 | CD28δ | CD79a |
| CD83 | CD86 | CD28δ | CD79b |
| CD83 | CD86 | CD80 | CD8 |
| CD83 | CD86 | CD80 | CD3ζ |
| CD83 | CD86 | CD80 | CD3δ |
| CD83 | CD86 | CD80 | CD3γ |
| CD83 | CD86 | CD80 | CD3ε |
| CD83 | CD86 | CD80 | FcγRI-γ |
| CD83 | CD86 | CD80 | FcγRIII-γ |
| CD83 | CD86 | CD80 | FcεRIβ |
| CD83 | CD86 | CD80 | FcεRIγ |
| CD83 | CD86 | CD80 | DAP10 |
| CD83 | CD86 | CD80 | DAP12 |
| CD83 | CD86 | CD80 | CD32 |
| CD83 | CD86 | CD80 | CD79a |
| CD83 | CD86 | CD80 | CD79b |
| CD83 | CD86 | CD86 | CD8 |
| CD83 | CD86 | CD86 | CD3ζ |
| CD83 | CD86 | CD86 | CD3δ |
| CD83 | CD86 | CD86 | CD3γ |
| CD83 | CD86 | CD86 | CD3ε |
| CD83 | CD86 | CD86 | FcγRI-γ |
| CD83 | CD86 | CD86 | FcγRIII-γ |
| CD83 | CD86 | CD86 | FcεRIβ |
| CD83 | CD86 | CD86 | FcεRIγ |
| CD83 | CD86 | CD86 | DAP10 |
| CD83 | CD86 | CD86 | DAP12 |
| CD83 | CD86 | CD86 | CD32 |
| CD83 | CD86 | CD86 | CD79a |
| CD83 | CD86 | CD86 | CD79b |
| CD83 | CD86 | OX40 | CD8 |
| CD83 | CD86 | OX40 | CD3ζ |
| CD83 | CD86 | OX40 | CD3δ |
| CD83 | CD86 | OX40 | CD3γ |
| CD83 | CD86 | OX40 | CD3ε |
| CD83 | CD86 | OX40 | FcγRI-γ |
| CD83 | CD86 | OX40 | FcγRIII-γ |
| CD83 | CD86 | OX40 | FcεRIβ |
| CD83 | CD86 | OX40 | FcεRIγ |
| CD83 | CD86 | OX40 | DAP10 |
| CD83 | CD86 | OX40 | DAP12 |
| CD83 | CD86 | OX40 | CD32 |
| CD83 | CD86 | OX40 | CD79a |
| CD83 | CD86 | OX40 | CD79b |
| CD83 | CD86 | DAP10 | CD8 |
| CD83 | CD86 | DAP10 | CD3ζ |
| CD83 | CD86 | DAP10 | CD3δ |
| CD83 | CD86 | DAP10 | CD3γ |
| CD83 | CD86 | DAP10 | CD3ε |
| CD83 | CD86 | DAP10 | FcγRI-γ |
| CD83 | CD86 | DAP10 | FcγRIII-γ |
| CD83 | CD86 | DAP10 | FcεRIβ |
| CD83 | CD86 | DAP10 | FcεRIγ |
| CD83 | CD86 | DAP10 | DAP10 |
| CD83 | CD86 | DAP10 | DAP12 |
| CD83 | CD86 | DAP10 | CD32 |
| CD83 | CD86 | DAP10 | CD79a |
| CD83 | CD86 | DAP10 | CD79b |
| CD83 | CD86 | DAP12 | CD8 |
| CD83 | CD86 | DAP12 | CD3ζ |
| CD83 | CD86 | DAP12 | CD3δ |
| CD83 | CD86 | DAP12 | CD3γ |
| CD83 | CD86 | DAP12 | CD3ε |
| CD83 | CD86 | DAP12 | FcγRI-γ |
| CD83 | CD86 | DAP12 | FcγRIII-γ |
| CD83 | CD86 | DAP12 | FcεRIβ |
| CD83 | CD86 | DAP12 | FcεRIγ |
| CD83 | CD86 | DAP12 | DAP10 |
| CD83 | CD86 | DAP12 | DAP12 |
| CD83 | CD86 | DAP12 | CD32 |
| CD83 | CD86 | DAP12 | CD79a |
| CD83 | CD86 | DAP12 | CD79b |
| CD83 | CD86 | MyD88 | CD8 |
| CD83 | CD86 | MyD88 | CD3ζ |
| CD83 | CD86 | MyD88 | CD3δ |
| CD83 | CD86 | MyD88 | CD3γ |
| CD83 | CD86 | MyD88 | CD3ε |
| CD83 | CD86 | MyD88 | FcγRI-γ |
| CD83 | CD86 | MyD88 | FcγRIII-γ |
| CD83 | CD86 | MyD88 | FcεRIβ |
| CD83 | CD86 | MyD88 | FcεRIγ |
| CD83 | CD86 | MyD88 | DAP10 |
| CD83 | CD86 | MyD88 | DAP12 |
| CD83 | CD86 | MyD88 | CD32 |
| CD83 | CD86 | MyD88 | CD79a |
| CD83 | CD86 | MyD88 | CD79b |
| CD83 | CD86 | CD7 | CD8 |
| CD83 | CD86 | CD7 | CD3ζ |
| CD83 | CD86 | CD7 | CD3δ |
| CD83 | CD86 | CD7 | CD3γ |
| CD83 | CD86 | CD7 | CD3ε |
| CD83 | CD86 | CD7 | FcγRI-γ |
| CD83 | CD86 | CD7 | FcγRIII-γ |
| CD83 | CD86 | CD7 | FcεRIβ |
| CD83 | CD86 | CD7 | FcεRIγ |
| CD83 | CD86 | CD7 | DAP10 |
| CD83 | CD86 | CD7 | DAP12 |
| CD83 | CD86 | CD7 | CD32 |
| CD83 | CD86 | CD7 | CD79a |
| CD83 | CD86 | CD7 | CD79b |
| CD83 | CD86 | BTNL3 | CD8 |
| CD83 | CD86 | BTNL3 | CD3ζ |
| CD83 | CD86 | BTNL3 | CD3δ |
| CD83 | CD86 | BTNL3 | CD3γ |
| CD83 | CD86 | BTNL3 | CD3ε |
| CD83 | CD86 | BTNL3 | FcγRI-γ |
| CD83 | CD86 | BTNL3 | FcγRIII-γ |
| CD83 | CD86 | BTNL3 | FcεRIβ |
| CD83 | CD86 | BTNL3 | FcεRIγ |
| CD83 | CD86 | BTNL3 | DAP10 |
| CD83 | CD86 | BTNL3 | DAP12 |
| CD83 | CD86 | BTNL3 | CD32 |
| CD83 | CD86 | BTNL3 | CD79a |
| CD83 | CD86 | BTNL3 | CD79b |
| CD83 | CD86 | NKG2D | CD8 |
| CD83 | CD86 | NKG2D | CD3ζ |
| CD83 | CD86 | NKG2D | CD3δ |
| CD83 | CD86 | NKG2D | CD3γ |
| CD83 | CD86 | NKG2D | CD3ε |
| CD83 | CD86 | NKG2D | FcγRI-γ |
| CD83 | CD86 | NKG2D | FcγRIII-γ |
| CD83 | CD86 | NKG2D | FcεRIβ |
| CD83 | CD86 | NKG2D | FcεRIγ |
| CD83 | CD86 | NKG2D | DAP10 |
| CD83 | CD86 | NKG2D | DAP12 |
| CD83 | CD86 | NKG2D | CD32 |
| CD83 | CD86 | NKG2D | CD79a |
| CD83 | CD86 | NKG2D | CD79b |
| CD83 | OX40 | CD28 | CD8 |
| CD83 | OX40 | CD28 | CD3ζ |
| CD83 | OX40 | CD28 | CD3δ |
| CD83 | OX40 | CD28 | CD3γ |
| CD83 | OX40 | CD28 | CD3ε |
| CD83 | OX40 | CD28 | FcγRI-γ |
| CD83 | OX40 | CD28 | FcγRIII-γ |
| CD83 | OX40 | CD28 | FcεRIβ |
| CD83 | OX40 | CD28 | FcεRIγ |
| CD83 | OX40 | CD28 | DAP10 |
| CD83 | OX40 | CD28 | DAP12 |
| CD83 | OX40 | CD28 | CD32 |
| CD83 | OX40 | CD28 | CD79a |
| CD83 | OX40 | CD28 | CD79b |
| CD83 | OX40 | CD8 | CD8 |
| CD83 | OX40 | CD8 | CD3ζ |
| CD83 | OX40 | CD8 | CD3δ |
| CD83 | OX40 | CD8 | CD3γ |
| CD83 | OX40 | CD8 | CD3ε |
| CD83 | OX40 | CD8 | FcγRI-γ |
| CD83 | OX40 | CD8 | FcγRIII-γ |
| CD83 | OX40 | CD8 | FcεRIβ |
| CD83 | OX40 | CD8 | FcεRIγ |
| CD83 | OX40 | CD8 | DAP10 |
| CD83 | OX40 | CD8 | DAP12 |
| CD83 | OX40 | CD8 | CD32 |
| CD83 | OX40 | CD8 | CD79a |
| CD83 | OX40 | CD8 | CD79b |
| CD83 | OX40 | CD4 | CD8 |
| CD83 | OX40 | CD4 | CD3ζ |
| CD83 | OX40 | CD4 | CD3δ |
| CD83 | OX40 | CD4 | CD3γ |
| CD83 | OX40 | CD4 | CD3ε |
| CD83 | OX40 | CD4 | FcγRI-γ |
| CD83 | OX40 | CD4 | FcγRIII-γ |
| CD83 | OX40 | CD4 | FcεRIβ |
| CD83 | OX40 | CD4 | FcεRIγ |
| CD83 | OX40 | CD4 | DAP10 |
| CD83 | OX40 | CD4 | DAP12 |
| CD83 | OX40 | CD4 | CD32 |
| CD83 | OX40 | CD4 | CD79a |
| CD83 | OX40 | CD4 | CD79b |
| CD83 | OX40 | b2c | CD8 |
| CD83 | OX40 | b2c | CD3ζ |
| CD83 | OX40 | b2c | CD3δ |
| CD83 | OX40 | b2c | CD3γ |
| CD83 | OX40 | b2c | CD3ε |
| CD83 | OX40 | b2c | FcγRI-γ |
| CD83 | OX40 | b2c | FcγRIII-γ |
| CD83 | OX40 | b2c | FcεRIβ |
| CD83 | OX40 | b2c | FcεRIγ |
| CD83 | OX40 | b2c | DAP10 |
| CD83 | OX40 | b2c | DAP12 |
| CD83 | OX40 | b2c | CD32 |
| CD83 | OX40 | b2c | CD79a |
| CD83 | OX40 | b2c | CD79b |
| CD83 | OX40 | CD137/41BB | CD8 |
| CD83 | OX40 | CD137/41BB | CD3ζ |
| CD83 | OX40 | CD137/41BB | CD3δ |
| CD83 | OX40 | CD137/41BB | CD3γ |
| CD83 | OX40 | CD137/41BB | CD3ε |
| CD83 | OX40 | CD137/41BB | FcγRI-γ |
| CD83 | OX40 | CD137/41BB | FcγRIII-γ |
| CD83 | OX40 | CD137/41BB | FcεRIβ |
| CD83 | OX40 | CD137/41BB | FcεRIγ |
| CD83 | OX40 | CD137/41BB | DAP10 |
| CD83 | OX40 | CD137/41BB | DAP12 |
| CD83 | OX40 | CD137/41BB | CD32 |
| CD83 | OX40 | CD137/41BB | CD79a |
| CD83 | OX40 | CD137/41BB | CD79b |
| CD83 | OX40 | ICOS | CD8 |
| CD83 | OX40 | ICOS | CD3ζ |
| CD83 | OX40 | ICOS | CD3δ |
| CD83 | OX40 | ICOS | CD3γ |
| CD83 | OX40 | ICOS | CD3ε |
| CD83 | OX40 | ICOS | FcγRI-γ |
| CD83 | OX40 | ICOS | FcγRIII-γ |
| CD83 | OX40 | ICOS | FcεRIβ |
| CD83 | OX40 | ICOS | FcεRIγ |
| CD83 | OX40 | ICOS | DAP10 |
| CD83 | OX40 | ICOS | DAP12 |
| CD83 | OX40 | ICOS | CD32 |
| CD83 | OX40 | ICOS | CD79a |
| CD83 | OX40 | ICOS | CD79b |
| CD83 | OX40 | CD27 | CD8 |
| CD83 | OX40 | CD27 | CD3ζ |
| CD83 | OX40 | CD27 | CD3δ |
| CD83 | OX40 | CD27 | CD3γ |
| CD83 | OX40 | CD27 | CD3ε |
| CD83 | OX40 | CD27 | FcγRI-γ |
| CD83 | OX40 | CD27 | FcγRIII-γ |
| CD83 | OX40 | CD27 | FcεRIβ |
| CD83 | OX40 | CD27 | FcεRIγ |
| CD83 | OX40 | CD27 | DAP10 |
| CD83 | OX40 | CD27 | DAP12 |
| CD83 | OX40 | CD27 | CD32 |
| CD83 | OX40 | CD27 | CD79a |
| CD83 | OX40 | CD27 | CD79b |
| CD83 | OX40 | CD28δ | CD8 |
| CD83 | OX40 | CD28δ | CD3ζ |
| CD83 | OX40 | CD28δ | CD3δ |
| CD83 | OX40 | CD28δ | CD3γ |
| CD83 | OX40 | CD28δ | CD3ε |
| CD83 | OX40 | CD28δ | FcγRI-γ |
| CD83 | OX40 | CD28δ | FcγRIII-γ |
| CD83 | OX40 | CD28δ | FcεRIβ |
| CD83 | OX40 | CD28δ | FcεRIγ |
| CD83 | OX40 | CD28δ | DAP10 |
| CD83 | OX40 | CD28δ | DAP12 |
| CD83 | OX40 | CD28δ | CD32 |
| CD83 | OX40 | CD28δ | CD79a |
| CD83 | OX40 | CD28δ | CD79b |
| CD83 | OX40 | CD80 | CD8 |
| CD83 | OX40 | CD80 | CD3ζ |
| CD83 | OX40 | CD80 | CD3δ |
| CD83 | OX40 | CD80 | CD3γ |
| CD83 | OX40 | CD80 | CD3ε |
| CD83 | OX40 | CD80 | FcγRI-γ |
| CD83 | OX40 | CD80 | FcγRIII-γ |
| CD83 | OX40 | CD80 | FcεRIβ |
| CD83 | OX40 | CD80 | FcεRIγ |
| CD83 | OX40 | CD80 | DAP10 |
| CD83 | OX40 | CD80 | DAP12 |
| CD83 | OX40 | CD80 | CD32 |
| CD83 | OX40 | CD80 | CD79a |
| CD83 | OX40 | CD80 | CD79b |
| CD83 | OX40 | CD86 | CD8 |
| CD83 | OX40 | CD86 | CD3ζ |
| CD83 | OX40 | CD86 | CD3δ |
| CD83 | OX40 | CD86 | CD3γ |
| CD83 | OX40 | CD86 | CD3ε |
| CD83 | OX40 | CD86 | FcγRI-γ |
| CD83 | OX40 | CD86 | FcγRIII-γ |
| CD83 | OX40 | CD86 | FcεRIβ |
| CD83 | OX40 | CD86 | FcεRIγ |
| CD83 | OX40 | CD86 | DAP10 |
| CD83 | OX40 | CD86 | DAP12 |
| CD83 | OX40 | CD86 | CD32 |
| CD83 | OX40 | CD86 | CD79a |
| CD83 | OX40 | CD86 | CD79b |
| CD83 | OX40 | OX40 | CD8 |
| CD83 | OX40 | OX40 | CD3ζ |
| CD83 | OX40 | OX40 | CD3δ |
| CD83 | OX40 | OX40 | CD3γ |
| CD83 | OX40 | OX40 | CD3ε |
| CD83 | OX40 | OX40 | FcγRI-γ |
| CD83 | OX40 | OX40 | FcγRIII-γ |
| CD83 | OX40 | OX40 | FcεRIβ |
| CD83 | OX40 | OX40 | FcεRIγ |
| CD83 | OX40 | OX40 | DAP10 |
| CD83 | OX40 | OX40 | DAP12 |
| CD83 | OX40 | OX40 | CD32 |
| CD83 | OX40 | OX40 | CD79a |
| CD83 | OX40 | OX40 | CD79b |
| CD83 | OX40 | DAP10 | CD8 |
| CD83 | OX40 | DAP10 | CD3ζ |
| CD83 | OX40 | DAP10 | CD3δ |
| CD83 | OX40 | DAP10 | CD3γ |
| CD83 | OX40 | DAP10 | CD3ε |
| CD83 | OX40 | DAP10 | FcγRI-γ |
| CD83 | OX40 | DAP10 | FcγRIII-γ |
| CD83 | OX40 | DAP10 | FcεRIβ |
| CD83 | OX40 | DAP10 | FcεRIγ |
| CD83 | OX40 | DAP10 | DAP10 |
| CD83 | OX40 | DAP10 | DAP12 |
| CD83 | OX40 | DAP10 | CD32 |
| CD83 | OX40 | DAP10 | CD79a |
| CD83 | OX40 | DAP10 | CD79b |
| CD83 | OX40 | DAP12 | CD8 |
| CD83 | OX40 | DAP12 | CD3ζ |
| CD83 | OX40 | DAP12 | CD3δ |
| CD83 | OX40 | DAP12 | CD3γ |
| CD83 | OX40 | DAP12 | CD3ε |
| CD83 | OX40 | DAP12 | FcγRI-γ |
| CD83 | OX40 | DAP12 | FcγRIII-γ |
| CD83 | OX40 | DAP12 | FcεRIβ |
| CD83 | OX40 | DAP12 | FcεRIγ |
| CD83 | OX40 | DAP12 | DAP10 |
| CD83 | OX40 | DAP12 | DAP12 |
| CD83 | OX40 | DAP12 | CD32 |
| CD83 | OX40 | DAP12 | CD79a |
| CD83 | OX40 | DAP12 | CD79b |
| CD83 | OX40 | MyD88 | CD8 |
| CD83 | OX40 | MyD88 | CD3ζ |
| CD83 | OX40 | MyD88 | CD3δ |
| CD83 | OX40 | MyD88 | CD3γ |
| CD83 | OX40 | MyD88 | CD3ε |
| CD83 | OX40 | MyD88 | FcγRI-γ |
| CD83 | OX40 | MyD88 | FcγRIII-γ |
| CD83 | OX40 | MyD88 | FcεRIβ |
| CD83 | OX40 | MyD88 | FcεRIγ |
| CD83 | OX40 | MyD88 | DAP10 |
| CD83 | OX40 | MyD88 | DAP12 |
| CD83 | OX40 | MyD88 | CD32 |
| CD83 | OX40 | MyD88 | CD79a |
| CD83 | OX40 | MyD88 | CD79b |
| CD83 | OX40 | CD7 | CD8 |
| CD83 | OX40 | CD7 | CD3ζ |
| CD83 | OX40 | CD7 | CD3δ |
| CD83 | OX40 | CD7 | CD3γ |
| CD83 | OX40 | CD7 | CD3ε |
| CD83 | OX40 | CD7 | FcγRI-γ |
| CD83 | OX40 | CD7 | FcγRIII-γ |
| CD83 | OX40 | CD7 | FcεRIβ |
| CD83 | OX40 | CD7 | FcεRIγ |
| CD83 | OX40 | CD7 | DAP10 |
| CD83 | OX40 | CD7 | DAP12 |
| CD83 | OX40 | CD7 | CD32 |
| CD83 | OX40 | CD7 | CD79a |
| CD83 | OX40 | CD7 | CD79b |
| CD83 | OX40 | BTNL3 | CD8 |
| CD83 | OX40 | BTNL3 | CD3ζ |
| CD83 | OX40 | BTNL3 | CD3δ |
| CD83 | OX40 | BTNL3 | CD3γ |
| CD83 | OX40 | BTNL3 | CD3ε |
| CD83 | OX40 | BTNL3 | FcγRI-γ |
| CD83 | OX40 | BTNL3 | FcγRIII-γ |
| CD83 | OX40 | BTNL3 | FcεRIβ |
| CD83 | OX40 | BTNL3 | FcεRIγ |
| CD83 | OX40 | BTNL3 | DAP10 |
| CD83 | OX40 | BTNL3 | DAP12 |
| CD83 | OX40 | BTNL3 | CD32 |
| CD83 | OX40 | BTNL3 | CD79a |
| CD83 | OX40 | BTNL3 | CD79b |
| CD83 | OX40 | NKG2D | CD8 |
| CD83 | OX40 | NKG2D | CD3ζ |
| CD83 | OX40 | NKG2D | CD3δ |
| CD83 | OX40 | NKG2D | CD3γ |
| CD83 | OX40 | NKG2D | CD3ε |
| CD83 | OX40 | NKG2D | FcγRI-γ |
| CD83 | OX40 | NKG2D | FcγRIII-γ |
| CD83 | OX40 | NKG2D | FcεRIβ |
| CD83 | OX40 | NKG2D | FcεRIγ |
| CD83 | OX40 | NKG2D | DAP10 |
| CD83 | OX40 | NKG2D | DAP12 |
| CD83 | OX40 | NKG2D | CD32 |
| CD83 | OX40 | NKG2D | CD79a |
| CD83 | OX40 | NKG2D | CD79b |
| CD83 | DAP10 | CD28 | CD8 |
| CD83 | DAP10 | CD28 | CD3ζ |
| CD83 | DAP10 | CD28 | CD3δ |
| CD83 | DAP10 | CD28 | CD3γ |
| CD83 | DAP10 | CD28 | CD3ε |
| CD83 | DAP10 | CD28 | FcγRI-γ |
| CD83 | DAP10 | CD28 | FcγRIII-γ |
| CD83 | DAP10 | CD28 | FcεRIβ |
| CD83 | DAP10 | CD28 | FcεRIγ |
| CD83 | DAP10 | CD28 | DAP10 |
| CD83 | DAP10 | CD28 | DAP12 |
| CD83 | DAP10 | CD28 | CD32 |
| CD83 | DAP10 | CD28 | CD79a |
| CD83 | DAP10 | CD28 | CD79b |
| CD83 | DAP10 | CD8 | CD8 |
| CD83 | DAP10 | CD8 | CD3ζ |
| CD83 | DAP10 | CD8 | CD3δ |
| CD83 | DAP10 | CD8 | CD3γ |
| CD83 | DAP10 | CD8 | CD3ε |
| CD83 | DAP10 | CD8 | FcγRI-γ |
| CD83 | DAP10 | CD8 | FcγRIII-γ |
| CD83 | DAP10 | CD8 | FcεRIβ |
| CD83 | DAP10 | CD8 | FcεRIγ |
| CD83 | DAP10 | CD8 | DAP10 |
| CD83 | DAP10 | CD8 | DAP12 |
| CD83 | DAP10 | CD8 | CD32 |
| CD83 | DAP10 | CD8 | CD79a |
| CD83 | DAP10 | CD8 | CD79b |
| CD83 | DAP10 | CD4 | CD8 |
| CD83 | DAP10 | CD4 | CD3ζ |
| CD83 | DAP10 | CD4 | CD3δ |
| CD83 | DAP10 | CD4 | CD3γ |
| CD83 | DAP10 | CD4 | CD3ε |
| CD83 | DAP10 | CD4 | FcγRI-γ |
| CD83 | DAP10 | CD4 | FcγRIII-γ |
| CD83 | DAP10 | CD4 | FcεRIβ |
| CD83 | DAP10 | CD4 | FcεRIγ |
| CD83 | DAP10 | CD4 | DAP10 |
| CD83 | DAP10 | CD4 | DAP12 |
| CD83 | DAP10 | CD4 | CD32 |
| CD83 | DAP10 | CD4 | CD79a |
| CD83 | DAP10 | CD4 | CD79b |
| CD83 | DAP10 | b2c | CD8 |
| CD83 | DAP10 | b2c | CD3ζ |
| CD83 | DAP10 | b2c | CD3δ |
| CD83 | DAP10 | b2c | CD3γ |
| CD83 | DAP10 | b2c | CD3ε |
| CD83 | DAP10 | b2c | FcγRI-γ |
| CD83 | DAP10 | b2c | FcγRIII-γ |
| CD83 | DAP10 | b2c | FcεRIβ |
| CD83 | DAP10 | b2c | FcεRIγ |
| CD83 | DAP10 | b2c | DAP10 |
| CD83 | DAP10 | b2c | DAP12 |
| CD83 | DAP10 | b2c | CD32 |
| CD83 | DAP10 | b2c | CD79a |
| CD83 | DAP10 | b2c | CD79b |
| CD83 | DAP10 | CD137/41BB | CD8 |
| CD83 | DAP10 | CD137/41BB | CD3ζ |
| CD83 | DAP10 | CD137/41BB | CD3δ |
| CD83 | DAP10 | CD137/41BB | CD3γ |
| CD83 | DAP10 | CD137/41BB | CD3ε |
| CD83 | DAP10 | CD137/41BB | FcγRI-γ |
| CD83 | DAP10 | CD137/41BB | FcγRIII-γ |
| CD83 | DAP10 | CD137/41BB | FcεRIβ |
| CD83 | DAP10 | CD137/41BB | FcεRIγ |
| CD83 | DAP10 | CD137/41BB | DAP10 |
| CD83 | DAP10 | CD137/41BB | DAP12 |
| CD83 | DAP10 | CD137/41BB | CD32 |
| CD83 | DAP10 | CD137/41BB | CD79a |
| CD83 | DAP10 | CD137/41BB | CD79b |
| CD83 | DAP10 | ICOS | CD8 |
| CD83 | DAP10 | ICOS | CD3ζ |
| CD83 | DAP10 | ICOS | CD3δ |
| CD83 | DAP10 | ICOS | CD3γ |
| CD83 | DAP10 | ICOS | CD3ε |
| CD83 | DAP10 | ICOS | FcγRI-γ |
| CD83 | DAP10 | ICOS | FcγRIII-γ |
| CD83 | DAP10 | ICOS | FcεRIβ |
| CD83 | DAP10 | ICOS | FcεRIγ |
| CD83 | DAP10 | ICOS | DAP10 |
| CD83 | DAP10 | ICOS | DAP12 |
| CD83 | DAP10 | ICOS | CD32 |
| CD83 | DAP10 | ICOS | CD79a |
| CD83 | DAP10 | ICOS | CD79b |
| CD83 | DAP10 | CD27 | CD8 |
| CD83 | DAP10 | CD27 | CD3ζ |
| CD83 | DAP10 | CD27 | CD3δ |
| CD83 | DAP10 | CD27 | CD3γ |
| CD83 | DAP10 | CD27 | CD3ε |
| CD83 | DAP10 | CD27 | FcγRI-γ |
| CD83 | DAP10 | CD27 | FcγRIII-γ |
| CD83 | DAP10 | CD27 | FcεRIβ |
| CD83 | DAP10 | CD27 | FcεRIγ |
| CD83 | DAP10 | CD27 | DAP10 |
| CD83 | DAP10 | CD27 | DAP12 |
| CD83 | DAP10 | CD27 | CD32 |
| CD83 | DAP10 | CD27 | CD79a |
| CD83 | DAP10 | CD27 | CD79b |
| CD83 | DAP10 | CD28δ | CD8 |
| CD83 | DAP10 | CD28δ | CD3ζ |
| CD83 | DAP10 | CD28δ | CD3δ |
| CD83 | DAP10 | CD28δ | CD3γ |
| CD83 | DAP10 | CD28δ | CD3ε |
| CD83 | DAP10 | CD28δ | FcγRI-γ |
| CD83 | DAP10 | CD28δ | FcγRIII-γ |
| CD83 | DAP10 | CD28δ | FcεRIβ |
| CD83 | DAP10 | CD28δ | FcεRIγ |
| CD83 | DAP10 | CD28δ | DAP10 |
| CD83 | DAP10 | CD28δ | DAP12 |
| CD83 | DAP10 | CD28δ | CD32 |
| CD83 | DAP10 | CD28δ | CD79a |
| CD83 | DAP10 | CD28δ | CD79b |
| CD83 | DAP10 | CD80 | CD8 |
| CD83 | DAP10 | CD80 | CD3ζ |
| CD83 | DAP10 | CD80 | CD3δ |
| CD83 | DAP10 | CD80 | CD3γ |
| CD83 | DAP10 | CD80 | CD3ε |
| CD83 | DAP10 | CD80 | FcγRI-γ |
| CD83 | DAP10 | CD80 | FcγRIII-γ |
| CD83 | DAP10 | CD80 | FcεRIβ |
| CD83 | DAP10 | CD80 | FcεRIγ |
| CD83 | DAP10 | CD80 | DAP10 |
| CD83 | DAP10 | CD80 | DAP12 |
| CD83 | DAP10 | CD80 | CD32 |
| CD83 | DAP10 | CD80 | CD79a |
| CD83 | DAP10 | CD80 | CD79b |
| CD83 | DAP10 | CD86 | CD8 |
| CD83 | DAP10 | CD86 | CD3ζ |
| CD83 | DAP10 | CD86 | CD3δ |
| CD83 | DAP10 | CD86 | CD3γ |
| CD83 | DAP10 | CD86 | CD3ε |
| CD83 | DAP10 | CD86 | FcγRI-γ |
| CD83 | DAP10 | CD86 | FcγRIII-γ |
| CD83 | DAP10 | CD86 | FcεRIβ |
| CD83 | DAP10 | CD86 | FcεRIγ |
| CD83 | DAP10 | CD86 | DAP10 |
| CD83 | DAP10 | CD86 | DAP12 |
| CD83 | DAP10 | CD86 | CD32 |
| CD83 | DAP10 | CD86 | CD79a |
| CD83 | DAP10 | CD86 | CD79b |
| CD83 | DAP10 | OX40 | CD8 |
| CD83 | DAP10 | OX40 | CD3ζ |
| CD83 | DAP10 | OX40 | CD3δ |
| CD83 | DAP10 | OX40 | CD3γ |
| CD83 | DAP10 | OX40 | CD3ε |
| CD83 | DAP10 | OX40 | FcγRI-γ |
| CD83 | DAP10 | OX40 | FcγRIII-γ |
| CD83 | DAP10 | OX40 | FcεRIβ |
| CD83 | DAP10 | OX40 | FcεRIγ |
| CD83 | DAP10 | OX40 | DAP10 |
| CD83 | DAP10 | OX40 | DAP12 |
| CD83 | DAP10 | OX40 | CD32 |
| CD83 | DAP10 | OX40 | CD79a |
| CD83 | DAP10 | OX40 | CD79b |
| CD83 | DAP10 | DAP10 | CD8 |
| CD83 | DAP10 | DAP10 | CD3ζ |
| CD83 | DAP10 | DAP10 | CD3δ |
| CD83 | DAP10 | DAP10 | CD3γ |
| CD83 | DAP10 | DAP10 | CD3ε |
| CD83 | DAP10 | DAP10 | FcγRI-γ |
| CD83 | DAP10 | DAP10 | FcγRIII-γ |
| CD83 | DAP10 | DAP10 | FcεRIβ |
| CD83 | DAP10 | DAP10 | FcεRIγ |
| CD83 | DAP10 | DAP10 | DAP10 |
| CD83 | DAP10 | DAP10 | DAP12 |
| CD83 | DAP10 | DAP10 | CD32 |
| CD83 | DAP10 | DAP10 | CD79a |
| CD83 | DAP10 | DAP10 | CD79b |
| CD83 | DAP10 | DAP12 | CD8 |
| CD83 | DAP10 | DAP12 | CD3ζ |
| CD83 | DAP10 | DAP12 | CD3δ |
| CD83 | DAP10 | DAP12 | CD3γ |
| CD83 | DAP10 | DAP12 | CD3ε |
| CD83 | DAP10 | DAP12 | FcγRI-γ |
| CD83 | DAP10 | DAP12 | FcγRIII-γ |
| CD83 | DAP10 | DAP12 | FcεRIβ |
| CD83 | DAP10 | DAP12 | FcεRIγ |
| CD83 | DAP10 | DAP12 | DAP10 |
| CD83 | DAP10 | DAP12 | DAP12 |
| CD83 | DAP10 | DAP12 | CD32 |
| CD83 | DAP10 | DAP12 | CD79a |
| CD83 | DAP10 | DAP12 | CD79b |
| CD83 | DAP10 | MyD88 | CD8 |
| CD83 | DAP10 | MyD88 | CD3ζ |
| CD83 | DAP10 | MyD88 | CD3δ |
| CD83 | DAP10 | MyD88 | CD3γ |
| CD83 | DAP10 | MyD88 | CD3ε |
| CD83 | DAP10 | MyD88 | FcγRI-γ |
| CD83 | DAP10 | MyD88 | FcγRIII-γ |
| CD83 | DAP10 | MyD88 | FcεRIβ |
| CD83 | DAP10 | MyD88 | FcεRIγ |
| CD83 | DAP10 | MyD88 | DAP10 |
| CD83 | DAP10 | MyD88 | DAP12 |
| CD83 | DAP10 | MyD88 | CD32 |
| CD83 | DAP10 | MyD88 | CD79a |
| CD83 | DAP10 | MyD88 | CD79b |
| CD83 | DAP10 | CD7 | CD8 |
| CD83 | DAP10 | CD7 | CD3ζ |
| CD83 | DAP10 | CD7 | CD3δ |
| CD83 | DAP10 | CD7 | CD3γ |
| CD83 | DAP10 | CD7 | CD3ε |
| CD83 | DAP10 | CD7 | FcγRI-γ |
| CD83 | DAP10 | CD7 | FcγRIII-γ |
| CD83 | DAP10 | CD7 | FcεRIβ |
| CD83 | DAP10 | CD7 | FcεRIγ |
| CD83 | DAP10 | CD7 | DAP10 |
| CD83 | DAP10 | CD7 | DAP12 |
| CD83 | DAP10 | CD7 | CD32 |
| CD83 | DAP10 | CD7 | CD79a |
| CD83 | DAP10 | CD7 | CD79b |
| CD83 | DAP10 | BTNL3 | CD8 |
| CD83 | DAP10 | BTNL3 | CD3ζ |
| CD83 | DAP10 | BTNL3 | CD3δ |
| CD83 | DAP10 | BTNL3 | CD3γ |
| CD83 | DAP10 | BTNL3 | CD3ε |
| CD83 | DAP10 | BTNL3 | FcγRI-γ |
| CD83 | DAP10 | BTNL3 | FcγRIII-γ |
| CD83 | DAP10 | BTNL3 | FcεRIβ |
| CD83 | DAP10 | BTNL3 | FcεRIγ |
| CD83 | DAP10 | BTNL3 | DAP10 |
| CD83 | DAP10 | BTNL3 | DAP12 |
| CD83 | DAP10 | BTNL3 | CD32 |
| CD83 | DAP10 | BTNL3 | CD79a |
| CD83 | DAP10 | BTNL3 | CD79b |
| CD83 | DAP10 | NKG2D | CD8 |
| CD83 | DAP10 | NKG2D | CD3ζ |
| CD83 | DAP10 | NKG2D | CD3δ |
| CD83 | DAP10 | NKG2D | CD3γ |
| CD83 | DAP10 | NKG2D | CD3ε |
| CD83 | DAP10 | NKG2D | FcγRI-γ |
| CD83 | DAP10 | NKG2D | FcγRIII-γ |
| CD83 | DAP10 | NKG2D | FcεRIβ |
| CD83 | DAP10 | NKG2D | FcεRIγ |
| CD83 | DAP10 | NKG2D | DAP10 |
| CD83 | DAP10 | NKG2D | DAP12 |
| CD83 | DAP10 | NKG2D | CD32 |
| CD83 | DAP10 | NKG2D | CD79a |
| CD83 | DAP10 | NKG2D | CD79b |
| CD83 | DAP12 | CD28 | CD8 |
| CD83 | DAP12 | CD28 | CD3ζ |
| CD83 | DAP12 | CD28 | CD3δ |
| CD83 | DAP12 | CD28 | CD3γ |
| CD83 | DAP12 | CD28 | CD3ε |
| CD83 | DAP12 | CD28 | FcγRI-γ |
| CD83 | DAP12 | CD28 | FcγRIII-γ |
| CD83 | DAP12 | CD28 | FcεRIβ |
| CD83 | DAP12 | CD28 | FcεRIγ |
| CD83 | DAP12 | CD28 | DAP10 |
| CD83 | DAP12 | CD28 | DAP12 |
| CD83 | DAP12 | CD28 | CD32 |
| CD83 | DAP12 | CD28 | CD79a |
| CD83 | DAP12 | CD28 | CD79b |
| CD83 | DAP12 | CD8 | CD8 |
| CD83 | DAP12 | CD8 | CD3ζ |
| CD83 | DAP12 | CD8 | CD3δ |
| CD83 | DAP12 | CD8 | CD3γ |
| CD83 | DAP12 | CD8 | CD3ε |
| CD83 | DAP12 | CD8 | FcγRI-γ |
| CD83 | DAP12 | CD8 | FcγRIII-γ |
| CD83 | DAP12 | CD8 | FcεRIβ |
| CD83 | DAP12 | CD8 | FcεRIγ |
| CD83 | DAP12 | CD8 | DAP10 |
| CD83 | DAP12 | CD8 | DAP12 |
| CD83 | DAP12 | CD8 | CD32 |
| CD83 | DAP12 | CD8 | CD79a |
| CD83 | DAP12 | CD8 | CD79b |
| CD83 | DAP12 | CD4 | CD8 |
| CD83 | DAP12 | CD4 | CD3ζ |
| CD83 | DAP12 | CD4 | CD3δ |
| CD83 | DAP12 | CD4 | CD3γ |
| CD83 | DAP12 | CD4 | CD3ε |
| CD83 | DAP12 | CD4 | FcγRI-γ |
| CD83 | DAP12 | CD4 | FcγRIII-γ |
| CD83 | DAP12 | CD4 | FcεRIβ |
| CD83 | DAP12 | CD4 | FcεRIγ |
| CD83 | DAP12 | CD4 | DAP10 |
| CD83 | DAP12 | CD4 | DAP12 |
| CD83 | DAP12 | CD4 | CD32 |
| CD83 | DAP12 | CD4 | CD79a |
| CD83 | DAP12 | CD4 | CD79b |
| CD83 | DAP12 | b2c | CD8 |
| CD83 | DAP12 | b2c | CD3ζ |
| CD83 | DAP12 | b2c | CD3δ |
| CD83 | DAP12 | b2c | CD3γ |
| CD83 | DAP12 | b2c | CD3ε |
| CD83 | DAP12 | b2c | FcγRI-γ |
| CD83 | DAP12 | b2c | FcγRIII-γ |
| CD83 | DAP12 | b2c | FcεRIβ |
| CD83 | DAP12 | b2c | FcεRIγ |
| CD83 | DAP12 | b2c | DAP10 |
| CD83 | DAP12 | b2c | DAP12 |
| CD83 | DAP12 | b2c | CD32 |
| CD83 | DAP12 | b2c | CD79a |
| CD83 | DAP12 | b2c | CD79b |
| CD83 | DAP12 | CD137/41BB | CD8 |
| CD83 | DAP12 | CD137/41BB | CD3ζ |
| CD83 | DAP12 | CD137/41BB | CD3δ |
| CD83 | DAP12 | CD137/41BB | CD3γ |
| CD83 | DAP12 | CD137/41BB | CD3ε |
| CD83 | DAP12 | CD137/41BB | FcγRI-γ |
| CD83 | DAP12 | CD137/41BB | FcγRIII-γ |
| CD83 | DAP12 | CD137/41BB | FcεRIβ |
| CD83 | DAP12 | CD137/41BB | FcεRIγ |
| CD83 | DAP12 | CD137/41BB | DAP10 |
| CD83 | DAP12 | CD137/41BB | DAP12 |
| CD83 | DAP12 | CD137/41BB | CD32 |
| CD83 | DAP12 | CD137/41BB | CD79a |
| CD83 | DAP12 | CD137/41BB | CD79b |
| CD83 | DAP12 | ICOS | CD8 |
| CD83 | DAP12 | ICOS | CD3ζ |
| CD83 | DAP12 | ICOS | CD3δ |
| CD83 | DAP12 | ICOS | CD3γ |
| CD83 | DAP12 | ICOS | CD3ε |
| CD83 | DAP12 | ICOS | FcγRI-γ |
| CD83 | DAP12 | ICOS | FcγRIII-γ |
| CD83 | DAP12 | ICOS | FcεRIβ |
| CD83 | DAP12 | ICOS | FcεRIγ |
| CD83 | DAP12 | ICOS | DAP10 |
| CD83 | DAP12 | ICOS | DAP12 |
| CD83 | DAP12 | ICOS | CD32 |
| CD83 | DAP12 | ICOS | CD79a |
| CD83 | DAP12 | ICOS | CD79b |
| CD83 | DAP12 | CD27 | CD8 |
| CD83 | DAP12 | CD27 | CD3ζ |
| CD83 | DAP12 | CD27 | CD3δ |
| CD83 | DAP12 | CD27 | CD3γ |
| CD83 | DAP12 | CD27 | CD3ε |
| CD83 | DAP12 | CD27 | FcγRI-γ |
| CD83 | DAP12 | CD27 | FcγRIII-γ |
| CD83 | DAP12 | CD27 | FcεRIβ |
| CD83 | DAP12 | CD27 | FcεRIγ |
| CD83 | DAP12 | CD27 | DAP10 |
| CD83 | DAP12 | CD27 | DAP12 |
| CD83 | DAP12 | CD27 | CD32 |
| CD83 | DAP12 | CD27 | CD79a |
| CD83 | DAP12 | CD27 | CD79b |
| CD83 | DAP12 | CD28δ | CD8 |
| CD83 | DAP12 | CD28δ | CD3ζ |
| CD83 | DAP12 | CD28δ | CD3δ |
| CD83 | DAP12 | CD28δ | CD3γ |
| CD83 | DAP12 | CD28δ | CD3ε |
| CD83 | DAP12 | CD28δ | FcγRI-γ |
| CD83 | DAP12 | CD28δ | FcγRIII-γ |
| CD83 | DAP12 | CD28δ | FcεRIβ |
| CD83 | DAP12 | CD28δ | FcεRIγ |
| CD83 | DAP12 | CD28δ | DAP10 |
| CD83 | DAP12 | CD28δ | DAP12 |
| CD83 | DAP12 | CD28δ | CD32 |
| CD83 | DAP12 | CD28δ | CD79a |
| CD83 | DAP12 | CD28δ | CD79b |
| CD83 | DAP12 | CD80 | CD8 |
| CD83 | DAP12 | CD80 | CD3ζ |
| CD83 | DAP12 | CD80 | CD3δ |
| CD83 | DAP12 | CD80 | CD3γ |
| CD83 | DAP12 | CD80 | CD3ε |
| CD83 | DAP12 | CD80 | FcγRI-γ |
| CD83 | DAP12 | CD80 | FcγRIII-γ |
| CD83 | DAP12 | CD80 | FcεRIβ |
| CD83 | DAP12 | CD80 | FcεRIγ |
| CD83 | DAP12 | CD80 | DAP10 |
| CD83 | DAP12 | CD80 | DAP12 |
| CD83 | DAP12 | CD80 | CD32 |
| CD83 | DAP12 | CD80 | CD79a |
| CD83 | DAP12 | CD80 | CD79b |
| CD83 | DAP12 | CD86 | CD8 |
| CD83 | DAP12 | CD86 | CD3ζ |
| CD83 | DAP12 | CD86 | CD3δ |
| CD83 | DAP12 | CD86 | CD3γ |
| CD83 | DAP12 | CD86 | CD3ε |
| CD83 | DAP12 | CD86 | FcγRI-γ |
| CD83 | DAP12 | CD86 | FcγRIII-γ |
| CD83 | DAP12 | CD86 | FcεRIβ |
| CD83 | DAP12 | CD86 | FcεRIγ |
| CD83 | DAP12 | CD86 | DAP10 |
| CD83 | DAP12 | CD86 | DAP12 |
| CD83 | DAP12 | CD86 | CD32 |
| CD83 | DAP12 | CD86 | CD79a |
| CD83 | DAP12 | CD86 | CD79b |
| CD83 | DAP12 | OX40 | CD8 |
| CD83 | DAP12 | OX40 | CD3ζ |
| CD83 | DAP12 | OX40 | CD3δ |
| CD83 | DAP12 | OX40 | CD3γ |
| CD83 | DAP12 | OX40 | CD3ε |
| CD83 | DAP12 | OX40 | FcγRI-γ |
| CD83 | DAP12 | OX40 | FcγRIII-γ |
| CD83 | DAP12 | OX40 | FcεRIβ |
| CD83 | DAP12 | OX40 | FcεRIγ |
| CD83 | DAP12 | OX40 | DAP10 |
| CD83 | DAP12 | OX40 | DAP12 |
| CD83 | DAP12 | OX40 | CD32 |
| CD83 | DAP12 | OX40 | CD79a |
| CD83 | DAP12 | OX40 | CD79b |
| CD83 | DAP12 | DAP10 | CD8 |
| CD83 | DAP12 | DAP10 | CD3ζ |
| CD83 | DAP12 | DAP10 | CD3δ |
| CD83 | DAP12 | DAP10 | CD3γ |
| CD83 | DAP12 | DAP10 | CD3ε |
| CD83 | DAP12 | DAP10 | FcγRI-γ |
| CD83 | DAP12 | DAP10 | FcγRIII-γ |
| CD83 | DAP12 | DAP10 | FcεRIβ |
| CD83 | DAP12 | DAP10 | FcεRIγ |
| CD83 | DAP12 | DAP10 | DAP10 |
| CD83 | DAP12 | DAP10 | DAP12 |
| CD83 | DAP12 | DAP10 | CD32 |
| CD83 | DAP12 | DAP10 | CD79a |
| CD83 | DAP12 | DAP10 | CD79b |
| CD83 | DAP12 | DAP12 | CD8 |
| CD83 | DAP12 | DAP12 | CD3ζ |
| CD83 | DAP12 | DAP12 | CD3δ |
| CD83 | DAP12 | DAP12 | CD3γ |
| CD83 | DAP12 | DAP12 | CD3ε |
| CD83 | DAP12 | DAP12 | FcγRI-γ |
| CD83 | DAP12 | DAP12 | FcγRIII-γ |
| CD83 | DAP12 | DAP12 | FcεRIβ |
| CD83 | DAP12 | DAP12 | FcεRIγ |
| CD83 | DAP12 | DAP12 | DAP10 |
| CD83 | DAP12 | DAP12 | DAP12 |
| CD83 | DAP12 | DAP12 | CD32 |
| CD83 | DAP12 | DAP12 | CD79a |
| CD83 | DAP12 | DAP12 | CD79b |
| CD83 | DAP12 | MyD88 | CD8 |
| CD83 | DAP12 | MyD88 | CD3ζ |
| CD83 | DAP12 | MyD88 | CD3δ |
| CD83 | DAP12 | MyD88 | CD3γ |
| CD83 | DAP12 | MyD88 | CD3ε |
| CD83 | DAP12 | MyD88 | FcγRI-γ |
| CD83 | DAP12 | MyD88 | FcγRIII-γ |
| CD83 | DAP12 | MyD88 | FcεRIβ |
| CD83 | DAP12 | MyD88 | FcεRIγ |
| CD83 | DAP12 | MyD88 | DAP10 |
| CD83 | DAP12 | MyD88 | DAP12 |
| CD83 | DAP12 | MyD88 | CD32 |
| CD83 | DAP12 | MyD88 | CD79a |
| CD83 | DAP12 | MyD88 | CD79b |
| CD83 | DAP12 | CD7 | CD8 |
| CD83 | DAP12 | CD7 | CD3ζ |
| CD83 | DAP12 | CD7 | CD3δ |
| CD83 | DAP12 | CD7 | CD3γ |
| CD83 | DAP12 | CD7 | CD3ε |
| CD83 | DAP12 | CD7 | FcγRI-γ |
| CD83 | DAP12 | CD7 | FcγRIII-γ |
| CD83 | DAP12 | CD7 | FcεRIβ |
| CD83 | DAP12 | CD7 | FcεRIγ |
| CD83 | DAP12 | CD7 | DAP10 |
| CD83 | DAP12 | CD7 | DAP12 |
| CD83 | DAP12 | CD7 | CD32 |
| CD83 | DAP12 | CD7 | CD79a |
| CD83 | DAP12 | CD7 | CD79b |
| CD83 | DAP12 | BTNL3 | CD8 |
| CD83 | DAP12 | BTNL3 | CD3ζ |
| CD83 | DAP12 | BTNL3 | CD3δ |
| CD83 | DAP12 | BTNL3 | CD3γ |
| CD83 | DAP12 | BTNL3 | CD3ε |
| CD83 | DAP12 | BTNL3 | FcγRI-γ |
| CD83 | DAP12 | BTNL3 | FcγRIII-γ |
| CD83 | DAP12 | BTNL3 | FcεRIβ |
| CD83 | DAP12 | BTNL3 | FcεRIγ |
| CD83 | DAP12 | BTNL3 | DAP10 |
| CD83 | DAP12 | BTNL3 | DAP12 |
| CD83 | DAP12 | BTNL3 | CD32 |
| CD83 | DAP12 | BTNL3 | CD79a |
| CD83 | DAP12 | BTNL3 | CD79b |
| CD83 | DAP12 | NKG2D | CD8 |
| CD83 | DAP12 | NKG2D | CD3ζ |
| CD83 | DAP12 | NKG2D | CD3δ |
| CD83 | DAP12 | NKG2D | CD3γ |
| CD83 | DAP12 | NKG2D | CD3ε |
| CD83 | DAP12 | NKG2D | FcγRI-γ |
| CD83 | DAP12 | NKG2D | FcγRIII-γ |
| CD83 | DAP12 | NKG2D | FcεRIβ |
| CD83 | DAP12 | NKG2D | FcεRIγ |
| CD83 | DAP12 | NKG2D | DAP10 |
| CD83 | DAP12 | NKG2D | DAP12 |
| CD83 | DAP12 | NKG2D | CD32 |
| CD83 | DAP12 | NKG2D | CD79a |
| CD83 | DAP12 | NKG2D | CD79b |
| CD83 | MyD88 | CD28 | CD8 |
| CD83 | MyD88 | CD28 | CD3ζ |
| CD83 | MyD88 | CD28 | CD3δ |
| CD83 | MyD88 | CD28 | CD3γ |
| CD83 | MyD88 | CD28 | CD3ε |
| CD83 | MyD88 | CD28 | FcγRI-γ |
| CD83 | MyD88 | CD28 | FcγRIII-γ |
| CD83 | MyD88 | CD28 | FcεRIβ |
| CD83 | MyD88 | CD28 | FcεRIγ |
| CD83 | MyD88 | CD28 | DAP10 |
| CD83 | MyD88 | CD28 | DAP12 |
| CD83 | MyD88 | CD28 | CD32 |
| CD83 | MyD88 | CD28 | CD79a |
| CD83 | MyD88 | CD28 | CD79b |
| CD83 | MyD88 | CD8 | CD8 |
| CD83 | MyD88 | CD8 | CD3ζ |
| CD83 | MyD88 | CD8 | CD3δ |
| CD83 | MyD88 | CD8 | CD3γ |
| CD83 | MyD88 | CD8 | CD3ε |
| CD83 | MyD88 | CD8 | FcγRI-γ |
| CD83 | MyD88 | CD8 | FcγRIII-γ |
| CD83 | MyD88 | CD8 | FcεRIβ |
| CD83 | MyD88 | CD8 | FcεRIγ |
| CD83 | MyD88 | CD8 | DAP10 |
| CD83 | MyD88 | CD8 | DAP12 |
| CD83 | MyD88 | CD8 | CD32 |
| CD83 | MyD88 | CD8 | CD79a |
| CD83 | MyD88 | CD8 | CD79b |
| CD83 | MyD88 | CD4 | CD8 |
| CD83 | MyD88 | CD4 | CD3ζ |
| CD83 | MyD88 | CD4 | CD3δ |
| CD83 | MyD88 | CD4 | CD3γ |
| CD83 | MyD88 | CD4 | CD3ε |
| CD83 | MyD88 | CD4 | FcγRI-γ |
| CD83 | MyD88 | CD4 | FcγRIII-γ |
| CD83 | MyD88 | CD4 | FcεRIβ |
| CD83 | MyD88 | CD4 | FcεRIγ |
| CD83 | MyD88 | CD4 | DAP10 |
| CD83 | MyD88 | CD4 | DAP12 |
| CD83 | MyD88 | CD4 | CD32 |
| CD83 | MyD88 | CD4 | CD79a |
| CD83 | MyD88 | CD4 | CD79b |
| CD83 | MyD88 | b2c | CD8 |
| CD83 | MyD88 | b2c | CD3ζ |
| CD83 | MyD88 | b2c | CD3δ |
| CD83 | MyD88 | b2c | CD3γ |
| CD83 | MyD88 | b2c | CD3ε |
| CD83 | MyD88 | b2c | FcγRI-γ |
| CD83 | MyD88 | b2c | FcγRIII-γ |
| CD83 | MyD88 | b2c | FcεRIβ |
| CD83 | MyD88 | b2c | FcεRIγ |
| CD83 | MyD88 | b2c | DAP10 |
| CD83 | MyD88 | b2c | DAP12 |
| CD83 | MyD88 | b2c | CD32 |
| CD83 | MyD88 | b2c | CD79a |
| CD83 | MyD88 | b2c | CD79b |
| CD83 | MyD88 | CD137/41BB | CD8 |
| CD83 | MyD88 | CD137/41BB | CD3ζ |
| CD83 | MyD88 | CD137/41BB | CD3δ |
| CD83 | MyD88 | CD137/41BB | CD3γ |
| CD83 | MyD88 | CD137/41BB | CD3ε |
| CD83 | MyD88 | CD137/41BB | FcγRI-γ |
| CD83 | MyD88 | CD137/41BB | FcγRIII-γ |
| CD83 | MyD88 | CD137/41BB | FcεRIβ |
| CD83 | MyD88 | CD137/41BB | FcεRIγ |
| CD83 | MyD88 | CD137/41BB | DAP10 |
| CD83 | MyD88 | CD137/41BB | DAP12 |
| CD83 | MyD88 | CD137/41BB | CD32 |
| CD83 | MyD88 | CD137/41BB | CD79a |
| CD83 | MyD88 | CD137/41BB | CD79b |
| CD83 | MyD88 | ICOS | CD8 |
| CD83 | MyD88 | ICOS | CD3ζ |
| CD83 | MyD88 | ICOS | CD3δ |
| CD83 | MyD88 | ICOS | CD3γ |
| CD83 | MyD88 | ICOS | CD3ε |
| CD83 | MyD88 | ICOS | FcγRI-γ |
| CD83 | MyD88 | ICOS | FcγRIII-γ |
| CD83 | MyD88 | ICOS | FcεRIβ |
| CD83 | MyD88 | ICOS | FcεRIγ |
| CD83 | MyD88 | ICOS | DAP10 |
| CD83 | MyD88 | ICOS | DAP12 |
| CD83 | MyD88 | ICOS | CD32 |
| CD83 | MyD88 | ICOS | CD79a |
| CD83 | MyD88 | ICOS | CD79b |
| CD83 | MyD88 | CD27 | CD8 |
| CD83 | MyD88 | CD27 | CD3ζ |
| CD83 | MyD88 | CD27 | CD3δ |
| CD83 | MyD88 | CD27 | CD3γ |
| CD83 | MyD88 | CD27 | CD3ε |
| CD83 | MyD88 | CD27 | FcγRI-γ |
| CD83 | MyD88 | CD27 | FcγRIII-γ |
| CD83 | MyD88 | CD27 | FcεRIβ |
| CD83 | MyD88 | CD27 | FcεRIγ |
| CD83 | MyD88 | CD27 | DAP10 |
| CD83 | MyD88 | CD27 | DAP12 |
| CD83 | MyD88 | CD27 | CD32 |
| CD83 | MyD88 | CD27 | CD79a |
| CD83 | MyD88 | CD27 | CD79b |
| CD83 | MyD88 | CD28δ | CD8 |
| CD83 | MyD88 | CD28δ | CD3ζ |
| CD83 | MyD88 | CD28δ | CD3δ |
| CD83 | MyD88 | CD28δ | CD3γ |
| CD83 | MyD88 | CD28δ | CD3ε |
| CD83 | MyD88 | CD28δ | FcγRI-γ |
| CD83 | MyD88 | CD28δ | FcγRIII-γ |
| CD83 | MyD88 | CD28δ | FcεRIβ |
| CD83 | MyD88 | CD28δ | FcεRIγ |
| CD83 | MyD88 | CD28δ | DAP10 |
| CD83 | MyD88 | CD28δ | DAP12 |
| CD83 | MyD88 | CD28δ | CD32 |
| CD83 | MyD88 | CD28δ | CD79a |
| CD83 | MyD88 | CD28δ | CD79b |
| CD83 | MyD88 | CD80 | CD8 |
| CD83 | MyD88 | CD80 | CD3ζ |
| CD83 | MyD88 | CD80 | CD3δ |
| CD83 | MyD88 | CD80 | CD3γ |
| CD83 | MyD88 | CD80 | CD3ε |
| CD83 | MyD88 | CD80 | FcγRI-γ |
| CD83 | MyD88 | CD80 | FcγRIII-γ |
| CD83 | MyD88 | CD80 | FcεRIβ |
| CD83 | MyD88 | CD80 | FcεRIγ |
| CD83 | MyD88 | CD80 | DAP10 |
| CD83 | MyD88 | CD80 | DAP12 |
| CD83 | MyD88 | CD80 | CD32 |
| CD83 | MyD88 | CD80 | CD79a |
| CD83 | MyD88 | CD80 | CD79b |
| CD83 | MyD88 | CD86 | CD8 |
| CD83 | MyD88 | CD86 | CD3ζ |
| CD83 | MyD88 | CD86 | CD3δ |
| CD83 | MyD88 | CD86 | CD3γ |
| CD83 | MyD88 | CD86 | CD3ε |
| CD83 | MyD88 | CD86 | FcγRI-γ |
| CD83 | MyD88 | CD86 | FcγRIII-γ |
| CD83 | MyD88 | CD86 | FcεRIβ |
| CD83 | MyD88 | CD86 | FcεRIγ |
| CD83 | MyD88 | CD86 | DAP10 |
| CD83 | MyD88 | CD86 | DAP12 |
| CD83 | MyD88 | CD86 | CD32 |
| CD83 | MyD88 | CD86 | CD79a |
| CD83 | MyD88 | CD86 | CD79b |
| CD83 | MyD88 | OX40 | CD8 |
| CD83 | MyD88 | OX40 | CD3ζ |
| CD83 | MyD88 | OX40 | CD3δ |
| CD83 | MyD88 | OX40 | CD3γ |
| CD83 | MyD88 | OX40 | CD3ε |
| CD83 | MyD88 | OX40 | FcγRI-γ |
| CD83 | MyD88 | OX40 | FcγRIII-γ |
| CD83 | MyD88 | OX40 | FcεRIβ |
| CD83 | MyD88 | OX40 | FcεRIγ |
| CD83 | MyD88 | OX40 | DAP10 |
| CD83 | MyD88 | OX40 | DAP12 |
| CD83 | MyD88 | OX40 | CD32 |
| CD83 | MyD88 | OX40 | CD79a |
| CD83 | MyD88 | OX40 | CD79b |
| CD83 | MyD88 | DAP10 | CD8 |
| CD83 | MyD88 | DAP10 | CD3ζ |
| CD83 | MyD88 | DAP10 | CD3δ |
| CD83 | MyD88 | DAP10 | CD3γ |
| CD83 | MyD88 | DAP10 | CD3ε |
| CD83 | MyD88 | DAP10 | FcγRI-γ |
| CD83 | MyD88 | DAP10 | FcγRIII-γ |
| CD83 | MyD88 | DAP10 | FcεRIβ |
| CD83 | MyD88 | DAP10 | FcεRIγ |
| CD83 | MyD88 | DAP10 | DAP10 |
| CD83 | MyD88 | DAP10 | DAP12 |
| CD83 | MyD88 | DAP10 | CD32 |
| CD83 | MyD88 | DAP10 | CD79a |
| CD83 | MyD88 | DAP10 | CD79b |
| CD83 | MyD88 | DAP12 | CD8 |
| CD83 | MyD88 | DAP12 | CD3ζ |
| CD83 | MyD88 | DAP12 | CD3δ |
| CD83 | MyD88 | DAP12 | CD3γ |
| CD83 | MyD88 | DAP12 | CD3ε |
| CD83 | MyD88 | DAP12 | FcγRI-γ |
| CD83 | MyD88 | DAP12 | FcγRIII-γ |
| CD83 | MyD88 | DAP12 | FcεRIβ |
| CD83 | MyD88 | DAP12 | FcεRIγ |
| CD83 | MyD88 | DAP12 | DAP10 |
| CD83 | MyD88 | DAP12 | DAP12 |
| CD83 | MyD88 | DAP12 | CD32 |
| CD83 | MyD88 | DAP12 | CD79a |
| CD83 | MyD88 | DAP12 | CD79b |
| CD83 | MyD88 | MyD88 | CD8 |
| CD83 | MyD88 | MyD88 | CD3ζ |
| CD83 | MyD88 | MyD88 | CD3δ |
| CD83 | MyD88 | MyD88 | CD3γ |
| CD83 | MyD88 | MyD88 | CD3ε |
| CD83 | MyD88 | MyD88 | FcγRI-γ |
| CD83 | MyD88 | MyD88 | FcγRIII-γ |
| CD83 | MyD88 | MyD88 | FcεRIβ |
| CD83 | MyD88 | MyD88 | FcεRIγ |
| CD83 | MyD88 | MyD88 | DAP10 |
| CD83 | MyD88 | MyD88 | DAP12 |
| CD83 | MyD88 | MyD88 | CD32 |
| CD83 | MyD88 | MyD88 | CD79a |
| CD83 | MyD88 | MyD88 | CD79b |
| CD83 | MyD88 | CD7 | CD8 |
| CD83 | MyD88 | CD7 | CD3ζ |
| CD83 | MyD88 | CD7 | CD3δ |
| CD83 | MyD88 | CD7 | CD3γ |
| CD83 | MyD88 | CD7 | CD3ε |
| CD83 | MyD88 | CD7 | FcγRI-γ |
| CD83 | MyD88 | CD7 | FcγRIII-γ |
| CD83 | MyD88 | CD7 | FcεRIβ |
| CD83 | MyD88 | CD7 | FcεRIγ |
| CD83 | MyD88 | CD7 | DAP10 |
| CD83 | MyD88 | CD7 | DAP12 |
| CD83 | MyD88 | CD7 | CD32 |
| CD83 | MyD88 | CD7 | CD79a |
| CD83 | MyD88 | CD7 | CD79b |
| CD83 | MyD88 | BTNL3 | CD8 |
| CD83 | MyD88 | BTNL3 | CD3ζ |
| CD83 | MyD88 | BTNL3 | CD3δ |
| CD83 | MyD88 | BTNL3 | CD3γ |
| CD83 | MyD88 | BTNL3 | CD3ε |
| CD83 | MyD88 | BTNL3 | FcγRI-γ |
| CD83 | MyD88 | BTNL3 | FcγRIII-γ |
| CD83 | MyD88 | BTNL3 | FcεRIβ |
| CD83 | MyD88 | BTNL3 | FcεRIγ |
| CD83 | MyD88 | BTNL3 | DAP10 |
| CD83 | MyD88 | BTNL3 | DAP12 |
| CD83 | MyD88 | BTNL3 | CD32 |
| CD83 | MyD88 | BTNL3 | CD79a |
| CD83 | MyD88 | BTNL3 | CD79b |
| CD83 | MyD88 | NKG2D | CD8 |
| CD83 | MyD88 | NKG2D | CD3ζ |
| CD83 | MyD88 | NKG2D | CD3δ |
| CD83 | MyD88 | NKG2D | CD3γ |
| CD83 | MyD88 | NKG2D | CD3ε |
| CD83 | MyD88 | NKG2D | FcγRI-γ |
| CD83 | MyD88 | NKG2D | FcγRIII-γ |
| CD83 | MyD88 | NKG2D | FcεRIβ |
| CD83 | MyD88 | NKG2D | FcεRIγ |
| CD83 | MyD88 | NKG2D | DAP10 |
| CD83 | MyD88 | NKG2D | DAP12 |
| CD83 | MyD88 | NKG2D | CD32 |
| CD83 | MyD88 | NKG2D | CD79a |
| CD83 | MyD88 | NKG2D | CD79b |
| CD83 | CD7 | CD28 | CD8 |
| CD83 | CD7 | CD28 | CD3ζ |
| CD83 | CD7 | CD28 | CD3δ |
| CD83 | CD7 | CD28 | CD3γ |
| CD83 | CD7 | CD28 | CD3ε |
| CD83 | CD7 | CD28 | FcγRI-γ |
| CD83 | CD7 | CD28 | FcγRIII-γ |
| CD83 | CD7 | CD28 | FcεRIβ |
| CD83 | CD7 | CD28 | FcεRIγ |
| CD83 | CD7 | CD28 | DAP10 |
| CD83 | CD7 | CD28 | DAP12 |
| CD83 | CD7 | CD28 | CD32 |
| CD83 | CD7 | CD28 | CD79a |
| CD83 | CD7 | CD28 | CD79b |
| CD83 | CD7 | CD8 | CD8 |
| CD83 | CD7 | CD8 | CD3ζ |
| CD83 | CD7 | CD8 | CD3δ |
| CD83 | CD7 | CD8 | CD3γ |
| CD83 | CD7 | CD8 | CD3ε |
| CD83 | CD7 | CD8 | FcγRI-γ |
| CD83 | CD7 | CD8 | FcγRIII-γ |
| CD83 | CD7 | CD8 | FcεRIβ |
| CD83 | CD7 | CD8 | FcεRIγ |
| CD83 | CD7 | CD8 | DAP10 |
| CD83 | CD7 | CD8 | DAP12 |
| CD83 | CD7 | CD8 | CD32 |
| CD83 | CD7 | CD8 | CD79a |
| CD83 | CD7 | CD8 | CD79b |
| CD83 | CD7 | CD4 | CD8 |
| CD83 | CD7 | CD4 | CD3ζ |
| CD83 | CD7 | CD4 | CD3δ |
| CD83 | CD7 | CD4 | CD3γ |
| CD83 | CD7 | CD4 | CD3ε |
| CD83 | CD7 | CD4 | FcγRI-γ |
| CD83 | CD7 | CD4 | FcγRIII-γ |
| CD83 | CD7 | CD4 | FcεRIβ |
| CD83 | CD7 | CD4 | FcεRIγ |
| CD83 | CD7 | CD4 | DAP10 |
| CD83 | CD7 | CD4 | DAP12 |
| CD83 | CD7 | CD4 | CD32 |
| CD83 | CD7 | CD4 | CD79a |
| CD83 | CD7 | CD4 | CD79b |
| CD83 | CD7 | b2c | CD8 |
| CD83 | CD7 | b2c | CD3ζ |
| CD83 | CD7 | b2c | CD3δ |
| CD83 | CD7 | b2c | CD3γ |
| CD83 | CD7 | b2c | CD3ε |
| CD83 | CD7 | b2c | FcγRI-γ |
| CD83 | CD7 | b2c | FcγRIII-γ |
| CD83 | CD7 | b2c | FcεRIβ |
| CD83 | CD7 | b2c | FcεRIγ |
| CD83 | CD7 | b2c | DAP10 |
| CD83 | CD7 | b2c | DAP12 |
| CD83 | CD7 | b2c | CD32 |
| CD83 | CD7 | b2c | CD79a |
| CD83 | CD7 | b2c | CD79b |
| CD83 | CD7 | CD137/41BB | CD8 |
| CD83 | CD7 | CD137/41BB | CD3ζ |
| CD83 | CD7 | CD137/41BB | CD3δ |
| CD83 | CD7 | CD137/41BB | CD3γ |
| CD83 | CD7 | CD137/41BB | CD3ε |
| CD83 | CD7 | CD137/41BB | FcγRI-γ |
| CD83 | CD7 | CD137/41BB | FcγRIII-γ |
| CD83 | CD7 | CD137/41BB | FcεRIβ |
| CD83 | CD7 | CD137/41BB | FcεRIγ |
| CD83 | CD7 | CD137/41BB | DAP10 |
| CD83 | CD7 | CD137/41BB | DAP12 |
| CD83 | CD7 | CD137/41BB | CD32 |
| CD83 | CD7 | CD137/41BB | CD79a |
| CD83 | CD7 | CD137/41BB | CD79b |
| CD83 | CD7 | ICOS | CD8 |
| CD83 | CD7 | ICOS | CD3ζ |
| CD83 | CD7 | ICOS | CD3δ |
| CD83 | CD7 | ICOS | CD3γ |
| CD83 | CD7 | ICOS | CD3ε |
| CD83 | CD7 | ICOS | FcγRI-γ |
| CD83 | CD7 | ICOS | FcγRIII-γ |
| CD83 | CD7 | ICOS | FcεRIβ |
| CD83 | CD7 | ICOS | FcεRIγ |
| CD83 | CD7 | ICOS | DAP10 |
| CD83 | CD7 | ICOS | DAP12 |
| CD83 | CD7 | ICOS | CD32 |
| CD83 | CD7 | ICOS | CD79a |
| CD83 | CD7 | ICOS | CD79b |
| CD83 | CD7 | CD27 | CD8 |
| CD83 | CD7 | CD27 | CD3ζ |
| CD83 | CD7 | CD27 | CD3δ |
| CD83 | CD7 | CD27 | CD3γ |
| CD83 | CD7 | CD27 | CD3ε |
| CD83 | CD7 | CD27 | FcγRI-γ |
| CD83 | CD7 | CD27 | FcγRIII-γ |
| CD83 | CD7 | CD27 | FcεRIβ |
| CD83 | CD7 | CD27 | FcεRIγ |
| CD83 | CD7 | CD27 | DAP10 |
| CD83 | CD7 | CD27 | DAP12 |
| CD83 | CD7 | CD27 | CD32 |
| CD83 | CD7 | CD27 | CD79a |
| CD83 | CD7 | CD27 | CD79b |
| CD83 | CD7 | CD28δ | CD8 |
| CD83 | CD7 | CD28δ | CD3ζ |
| CD83 | CD7 | CD28δ | CD3δ |
| CD83 | CD7 | CD28δ | CD3γ |
| CD83 | CD7 | CD28δ | CD3ε |
| CD83 | CD7 | CD28δ | FcγRI-γ |
| CD83 | CD7 | CD28δ | FcγRIII-γ |
| CD83 | CD7 | CD28δ | FcεRIβ |
| CD83 | CD7 | CD28δ | FcεRIγ |
| CD83 | CD7 | CD28δ | DAP10 |
| CD83 | CD7 | CD28δ | DAP12 |
| CD83 | CD7 | CD28δ | CD32 |
| CD83 | CD7 | CD28δ | CD79a |
| CD83 | CD7 | CD28δ | CD79b |
| CD83 | CD7 | CD80 | CD8 |
| CD83 | CD7 | CD80 | CD3ζ |
| CD83 | CD7 | CD80 | CD3δ |
| CD83 | CD7 | CD80 | CD3γ |
| CD83 | CD7 | CD80 | CD3ε |
| CD83 | CD7 | CD80 | FcγRI-γ |
| CD83 | CD7 | CD80 | FcγRIII-γ |
| CD83 | CD7 | CD80 | FcεRIβ |
| CD83 | CD7 | CD80 | FcεRIγ |
| CD83 | CD7 | CD80 | DAP10 |
| CD83 | CD7 | CD80 | DAP12 |
| CD83 | CD7 | CD80 | CD32 |
| CD83 | CD7 | CD80 | CD79a |
| CD83 | CD7 | CD80 | CD79b |
| CD83 | CD7 | CD86 | CD8 |
| CD83 | CD7 | CD86 | CD3ζ |
| CD83 | CD7 | CD86 | CD3δ |
| CD83 | CD7 | CD86 | CD3γ |
| CD83 | CD7 | CD86 | CD3ε |
| CD83 | CD7 | CD86 | FcγRI-γ |
| CD83 | CD7 | CD86 | FcγRIII-γ |
| CD83 | CD7 | CD86 | FcεRIβ |
| CD83 | CD7 | CD86 | FcεRIγ |
| CD83 | CD7 | CD86 | DAP10 |
| CD83 | CD7 | CD86 | DAP12 |
| CD83 | CD7 | CD86 | CD32 |
| CD83 | CD7 | CD86 | CD79a |
| CD83 | CD7 | CD86 | CD79b |
| CD83 | CD7 | OX40 | CD8 |
| CD83 | CD7 | OX40 | CD3ζ |
| CD83 | CD7 | OX40 | CD3δ |
| CD83 | CD7 | OX40 | CD3γ |
| CD83 | CD7 | OX40 | CD3ε |
| CD83 | CD7 | OX40 | FcγRI-γ |
| CD83 | CD7 | OX40 | FcγRIII-γ |
| CD83 | CD7 | OX40 | FcεRIβ |
| CD83 | CD7 | OX40 | FcεRIγ |
| CD83 | CD7 | OX40 | DAP10 |
| CD83 | CD7 | OX40 | DAP12 |
| CD83 | CD7 | OX40 | CD32 |
| CD83 | CD7 | OX40 | CD79a |
| CD83 | CD7 | OX40 | CD79b |
| CD83 | CD7 | DAP10 | CD8 |
| CD83 | CD7 | DAP10 | CD3ζ |
| CD83 | CD7 | DAP10 | CD3δ |
| CD83 | CD7 | DAP10 | CD3γ |
| CD83 | CD7 | DAP10 | CD3ε |
| CD83 | CD7 | DAP10 | FcγRI-γ |
| CD83 | CD7 | DAP10 | FcγRIII-γ |
| CD83 | CD7 | DAP10 | FcεRIβ |
| CD83 | CD7 | DAP10 | FcεRIγ |
| CD83 | CD7 | DAP10 | DAP10 |
| CD83 | CD7 | DAP10 | DAP12 |
| CD83 | CD7 | DAP10 | CD32 |
| CD83 | CD7 | DAP10 | CD79a |
| CD83 | CD7 | DAP10 | CD79b |
| CD83 | CD7 | DAP12 | CD8 |
| CD83 | CD7 | DAP12 | CD3ζ |
| CD83 | CD7 | DAP12 | CD3δ |
| CD83 | CD7 | DAP12 | CD3γ |
| CD83 | CD7 | DAP12 | CD3ε |
| CD83 | CD7 | DAP12 | FcγRI-γ |
| CD83 | CD7 | DAP12 | FcγRIII-γ |
| CD83 | CD7 | DAP12 | FcεRIβ |
| CD83 | CD7 | DAP12 | FcεRIγ |
| CD83 | CD7 | DAP12 | DAP10 |
| CD83 | CD7 | DAP12 | DAP12 |
| CD83 | CD7 | DAP12 | CD32 |
| CD83 | CD7 | DAP12 | CD79a |
| CD83 | CD7 | DAP12 | CD79b |
| CD83 | CD7 | MyD88 | CD8 |
| CD83 | CD7 | MyD88 | CD3ζ |
| CD83 | CD7 | MyD88 | CD3δ |
| CD83 | CD7 | MyD88 | CD3γ |
| CD83 | CD7 | MyD88 | CD3ε |
| CD83 | CD7 | MyD88 | FcγRI-γ |
| CD83 | CD7 | MyD88 | FcγRIII-γ |
| CD83 | CD7 | MyD88 | FcεRIβ |
| CD83 | CD7 | MyD88 | FcεRIγ |
| CD83 | CD7 | MyD88 | DAP10 |
| CD83 | CD7 | MyD88 | DAP12 |
| CD83 | CD7 | MyD88 | CD32 |
| CD83 | CD7 | MyD88 | CD79a |
| CD83 | CD7 | MyD88 | CD79b |
| CD83 | CD7 | CD7 | CD8 |
| CD83 | CD7 | CD7 | CD3ζ |
| CD83 | CD7 | CD7 | CD3δ |
| CD83 | CD7 | CD7 | CD3γ |
| CD83 | CD7 | CD7 | CD3ε |
| CD83 | CD7 | CD7 | FcγRI-γ |
| CD83 | CD7 | CD7 | FcγRIII-γ |
| CD83 | CD7 | CD7 | FcεRIβ |
| CD83 | CD7 | CD7 | FcεRIγ |
| CD83 | CD7 | CD7 | DAP10 |
| CD83 | CD7 | CD7 | DAP12 |
| CD83 | CD7 | CD7 | CD32 |
| CD83 | CD7 | CD7 | CD79a |
| CD83 | CD7 | CD7 | CD79b |
| CD83 | CD7 | BTNL3 | CD8 |
| CD83 | CD7 | BTNL3 | CD3ζ |
| CD83 | CD7 | BTNL3 | CD3δ |
| CD83 | CD7 | BTNL3 | CD3γ |
| CD83 | CD7 | BTNL3 | CD3ε |
| CD83 | CD7 | BTNL3 | FcγRI-γ |
| CD83 | CD7 | BTNL3 | FcγRIII-γ |
| CD83 | CD7 | BTNL3 | FcεRIβ |
| CD83 | CD7 | BTNL3 | FcεRIγ |
| CD83 | CD7 | BTNL3 | DAP10 |
| CD83 | CD7 | BTNL3 | DAP12 |
| CD83 | CD7 | BTNL3 | CD32 |
| CD83 | CD7 | BTNL3 | CD79a |
| CD83 | CD7 | BTNL3 | CD79b |
| CD83 | CD7 | NKG2D | CD8 |
| CD83 | CD7 | NKG2D | CD3ζ |
| CD83 | CD7 | NKG2D | CD3δ |
| CD83 | CD7 | NKG2D | CD3γ |
| CD83 | CD7 | NKG2D | CD3ε |
| CD83 | CD7 | NKG2D | FcγRI-γ |
| CD83 | CD7 | NKG2D | FcγRIII-γ |
| CD83 | CD7 | NKG2D | FcεRIβ |
| CD83 | CD7 | NKG2D | FcεRIγ |
| CD83 | CD7 | NKG2D | DAP10 |
| CD83 | CD7 | NKG2D | DAP12 |
| CD83 | CD7 | NKG2D | CD32 |
| CD83 | CD7 | NKG2D | CD79a |
| CD83 | CD7 | NKG2D | CD79b |
| CD83 | BTNL3 | CD28 | CD8 |
| CD83 | BTNL3 | CD28 | CD3ζ |
| CD83 | BTNL3 | CD28 | CD3δ |
| CD83 | BTNL3 | CD28 | CD3γ |
| CD83 | BTNL3 | CD28 | CD3ε |
| CD83 | BTNL3 | CD28 | FcγRI-γ |
| CD83 | BTNL3 | CD28 | FcγRIII-γ |
| CD83 | BTNL3 | CD28 | FcεRIβ |
| CD83 | BTNL3 | CD28 | FcεRIγ |
| CD83 | BTNL3 | CD28 | DAP10 |
| CD83 | BTNL3 | CD28 | DAP12 |
| CD83 | BTNL3 | CD28 | CD32 |
| CD83 | BTNL3 | CD28 | CD79a |
| CD83 | BTNL3 | CD28 | CD79b |
| CD83 | BTNL3 | CD8 | CD8 |
| CD83 | BTNL3 | CD8 | CD3ζ |
| CD83 | BTNL3 | CD8 | CD3δ |
| CD83 | BTNL3 | CD8 | CD3γ |
| CD83 | BTNL3 | CD8 | CD3ε |
| CD83 | BTNL3 | CD8 | FcγRI-γ |
| CD83 | BTNL3 | CD8 | FcγRIII-γ |
| CD83 | BTNL3 | CD8 | FcεRIβ |
| CD83 | BTNL3 | CD8 | FcεRIγ |
| CD83 | BTNL3 | CD8 | DAP10 |
| CD83 | BTNL3 | CD8 | DAP12 |
| CD83 | BTNL3 | CD8 | CD32 |
| CD83 | BTNL3 | CD8 | CD79a |
| CD83 | BTNL3 | CD8 | CD79b |
| CD83 | BTNL3 | CD4 | CD8 |
| CD83 | BTNL3 | CD4 | CD3ζ |
| CD83 | BTNL3 | CD4 | CD3δ |
| CD83 | BTNL3 | CD4 | CD3γ |
| CD83 | BTNL3 | CD4 | CD3ε |
| CD83 | BTNL3 | CD4 | FcγRI-γ |
| CD83 | BTNL3 | CD4 | FcγRIII-γ |
| CD83 | BTNL3 | CD4 | FcεRIβ |
| CD83 | BTNL3 | CD4 | FcεRIγ |
| CD83 | BTNL3 | CD4 | DAP10 |
| CD83 | BTNL3 | CD4 | DAP12 |
| CD83 | BTNL3 | CD4 | CD32 |
| CD83 | BTNL3 | CD4 | CD79a |
| CD83 | BTNL3 | CD4 | CD79b |
| CD83 | BTNL3 | b2c | CD8 |
| CD83 | BTNL3 | b2c | CD3ζ |
| CD83 | BTNL3 | b2c | CD3δ |
| CD83 | BTNL3 | b2c | CD3γ |
| CD83 | BTNL3 | b2c | CD3ε |
| CD83 | BTNL3 | b2c | FcγRI-γ |
| CD83 | BTNL3 | b2c | FcγRIII-γ |
| CD83 | BTNL3 | b2c | FcεRIβ |
| CD83 | BTNL3 | b2c | FcεRIγ |
| CD83 | BTNL3 | b2c | DAP10 |
| CD83 | BTNL3 | b2c | DAP12 |
| CD83 | BTNL3 | b2c | CD32 |
| CD83 | BTNL3 | b2c | CD79a |
| CD83 | BTNL3 | b2c | CD79b |
| CD83 | BTNL3 | CD137/41BB | CD8 |
| CD83 | BTNL3 | CD137/41BB | CD3ζ |
| CD83 | BTNL3 | CD137/41BB | CD3δ |
| CD83 | BTNL3 | CD137/41BB | CD3γ |
| CD83 | BTNL3 | CD137/41BB | CD3ε |
| CD83 | BTNL3 | CD137/41BB | FcγRI-γ |
| CD83 | BTNL3 | CD137/41BB | FcγRIII-γ |
| CD83 | BTNL3 | CD137/41BB | FcεRIβ |
| CD83 | BTNL3 | CD137/41BB | FcεRIγ |
| CD83 | BTNL3 | CD137/41BB | DAP10 |
| CD83 | BTNL3 | CD137/41BB | DAP12 |
| CD83 | BTNL3 | CD137/41BB | CD32 |
| CD83 | BTNL3 | CD137/41BB | CD79a |
| CD83 | BTNL3 | CD137/41BB | CD79b |
| CD83 | BTNL3 | ICOS | CD8 |
| CD83 | BTNL3 | ICOS | CD3ζ |
| CD83 | BTNL3 | ICOS | CD3δ |
| CD83 | BTNL3 | ICOS | CD3γ |
| CD83 | BTNL3 | ICOS | CD3ε |
| CD83 | BTNL3 | ICOS | FcγRI-γ |
| CD83 | BTNL3 | ICOS | FcγRIII-γ |
| CD83 | BTNL3 | ICOS | FcεRIβ |
| CD83 | BTNL3 | ICOS | FcεRIγ |
| CD83 | BTNL3 | ICOS | DAP10 |
| CD83 | BTNL3 | ICOS | DAP12 |
| CD83 | BTNL3 | ICOS | CD32 |
| CD83 | BTNL3 | ICOS | CD79a |
| CD83 | BTNL3 | ICOS | CD79b |
| CD83 | BTNL3 | CD27 | CD8 |
| CD83 | BTNL3 | CD27 | CD3ζ |
| CD83 | BTNL3 | CD27 | CD3δ |
| CD83 | BTNL3 | CD27 | CD3γ |
| CD83 | BTNL3 | CD27 | CD3ε |
| CD83 | BTNL3 | CD27 | FcγRI-γ |
| CD83 | BTNL3 | CD27 | FcγRIII-γ |
| CD83 | BTNL3 | CD27 | FcεRIβ |
| CD83 | BTNL3 | CD27 | FcεRIγ |
| CD83 | BTNL3 | CD27 | DAP10 |
| CD83 | BTNL3 | CD27 | DAP12 |
| CD83 | BTNL3 | CD27 | CD32 |
| CD83 | BTNL3 | CD27 | CD79a |
| CD83 | BTNL3 | CD27 | CD79b |
| CD83 | BTNL3 | CD28δ | CD8 |
| CD83 | BTNL3 | CD28δ | CD3ζ |
| CD83 | BTNL3 | CD28δ | CD3δ |
| CD83 | BTNL3 | CD28δ | CD3γ |
| CD83 | BTNL3 | CD28δ | CD3ε |
| CD83 | BTNL3 | CD28δ | FcγRI-γ |
| CD83 | BTNL3 | CD28δ | FcγRIII-γ |
| CD83 | BTNL3 | CD28δ | FcεRIβ |
| CD83 | BTNL3 | CD28δ | FcεRIγ |
| CD83 | BTNL3 | CD28δ | DAP10 |
| CD83 | BTNL3 | CD28δ | DAP12 |
| CD83 | BTNL3 | CD28δ | CD32 |
| CD83 | BTNL3 | CD28δ | CD79a |
| CD83 | BTNL3 | CD28δ | CD79b |
| CD83 | BTNL3 | CD80 | CD8 |
| CD83 | BTNL3 | CD80 | CD3ζ |
| CD83 | BTNL3 | CD80 | CD3δ |
| CD83 | BTNL3 | CD80 | CD3γ |
| CD83 | BTNL3 | CD80 | CD3ε |
| CD83 | BTNL3 | CD80 | FcγRI-γ |
| CD83 | BTNL3 | CD80 | FcγRIII-γ |
| CD83 | BTNL3 | CD80 | FcεRIβ |
| CD83 | BTNL3 | CD80 | FcεRIγ |
| CD83 | BTNL3 | CD80 | DAP10 |
| CD83 | BTNL3 | CD80 | DAP12 |
| CD83 | BTNL3 | CD80 | CD32 |
| CD83 | BTNL3 | CD80 | CD79a |
| CD83 | BTNL3 | CD80 | CD79b |
| CD83 | BTNL3 | CD86 | CD8 |
| CD83 | BTNL3 | CD86 | CD3ζ |
| CD83 | BTNL3 | CD86 | CD3δ |
| CD83 | BTNL3 | CD86 | CD3γ |
| CD83 | BTNL3 | CD86 | CD3ε |
| CD83 | BTNL3 | CD86 | FcγRI-γ |
| CD83 | BTNL3 | CD86 | FcγRIII-γ |
| CD83 | BTNL3 | CD86 | FcεRIβ |
| CD83 | BTNL3 | CD86 | FcεRIγ |
| CD83 | BTNL3 | CD86 | DAP10 |
| CD83 | BTNL3 | CD86 | DAP12 |
| CD83 | BTNL3 | CD86 | CD32 |
| CD83 | BTNL3 | CD86 | CD79a |
| CD83 | BTNL3 | CD86 | CD79b |
| CD83 | BTNL3 | OX40 | CD8 |
| CD83 | BTNL3 | OX40 | CD3ζ |
| CD83 | BTNL3 | OX40 | CD3δ |
| CD83 | BTNL3 | OX40 | CD3γ |
| CD83 | BTNL3 | OX40 | CD3ε |
| CD83 | BTNL3 | OX40 | FcγRI-γ |
| CD83 | BTNL3 | OX40 | FcγRIII-γ |
| CD83 | BTNL3 | OX40 | FcεRIβ |
| CD83 | BTNL3 | OX40 | FcεRIγ |
| CD83 | BTNL3 | OX40 | DAP10 |
| CD83 | BTNL3 | OX40 | DAP12 |
| CD83 | BTNL3 | OX40 | CD32 |
| CD83 | BTNL3 | OX40 | CD79a |
| CD83 | BTNL3 | OX40 | CD79b |
| CD83 | BTNL3 | DAP10 | CD8 |
| CD83 | BTNL3 | DAP10 | CD3ζ |
| CD83 | BTNL3 | DAP10 | CD3δ |
| CD83 | BTNL3 | DAP10 | CD3γ |
| CD83 | BTNL3 | DAP10 | CD3ε |
| CD83 | BTNL3 | DAP10 | FcγRI-γ |
| CD83 | BTNL3 | DAP10 | FcγRIII-γ |
| CD83 | BTNL3 | DAP10 | FcεRIβ |
| CD83 | BTNL3 | DAP10 | FcεRIγ |
| CD83 | BTNL3 | DAP10 | DAP10 |
| CD83 | BTNL3 | DAP10 | DAP12 |
| CD83 | BTNL3 | DAP10 | CD32 |
| CD83 | BTNL3 | DAP10 | CD79a |
| CD83 | BTNL3 | DAP10 | CD79b |
| CD83 | BTNL3 | DAP12 | CD8 |
| CD83 | BTNL3 | DAP12 | CD3ζ |
| CD83 | BTNL3 | DAP12 | CD3δ |
| CD83 | BTNL3 | DAP12 | CD3γ |
| CD83 | BTNL3 | DAP12 | CD3ε |
| CD83 | BTNL3 | DAP12 | FcγRI-γ |
| CD83 | BTNL3 | DAP12 | FcγRIII-γ |
| CD83 | BTNL3 | DAP12 | FcεRIβ |
| CD83 | BTNL3 | DAP12 | FcεRIγ |
| CD83 | BTNL3 | DAP12 | DAP10 |
| CD83 | BTNL3 | DAP12 | DAP12 |
| CD83 | BTNL3 | DAP12 | CD32 |
| CD83 | BTNL3 | DAP12 | CD79a |
| CD83 | BTNL3 | DAP12 | CD79b |
| CD83 | BTNL3 | MyD88 | CD8 |
| CD83 | BTNL3 | MyD88 | CD3ζ |
| CD83 | BTNL3 | MyD88 | CD3δ |
| CD83 | BTNL3 | MyD88 | CD3γ |
| CD83 | BTNL3 | MyD88 | CD3ε |
| CD83 | BTNL3 | MyD88 | FcγRI-γ |
| CD83 | BTNL3 | MyD88 | FcγRIII-γ |
| CD83 | BTNL3 | MyD88 | FcεRIβ |
| CD83 | BTNL3 | MyD88 | FcεRIγ |
| CD83 | BTNL3 | MyD88 | DAP10 |
| CD83 | BTNL3 | MyD88 | DAP12 |
| CD83 | BTNL3 | MyD88 | CD32 |
| CD83 | BTNL3 | MyD88 | CD79a |
| CD83 | BTNL3 | MyD88 | CD79b |
| CD83 | BTNL3 | CD7 | CD8 |
| CD83 | BTNL3 | CD7 | CD3ζ |
| CD83 | BTNL3 | CD7 | CD3δ |
| CD83 | BTNL3 | CD7 | CD3γ |
| CD83 | BTNL3 | CD7 | CD3ε |
| CD83 | BTNL3 | CD7 | FcγRI-γ |
| CD83 | BTNL3 | CD7 | FcγRIII-γ |
| CD83 | BTNL3 | CD7 | FcεRIβ |
| CD83 | BTNL3 | CD7 | FcεRIγ |
| CD83 | BTNL3 | CD7 | DAP10 |
| CD83 | BTNL3 | CD7 | DAP12 |
| CD83 | BTNL3 | CD7 | CD32 |
| CD83 | BTNL3 | CD7 | CD79a |
| CD83 | BTNL3 | CD7 | CD79b |
| CD83 | BTNL3 | BTNL3 | CD8 |
| CD83 | BTNL3 | BTNL3 | CD3ζ |
| CD83 | BTNL3 | BTNL3 | CD3δ |
| CD83 | BTNL3 | BTNL3 | CD3γ |
| CD83 | BTNL3 | BTNL3 | CD3ε |
| CD83 | BTNL3 | BTNL3 | FcγRI-γ |
| CD83 | BTNL3 | BTNL3 | FcγRIII-γ |
| CD83 | BTNL3 | BTNL3 | FcεRIβ |
| CD83 | BTNL3 | BTNL3 | FcεRIγ |
| CD83 | BTNL3 | BTNL3 | DAP10 |
| CD83 | BTNL3 | BTNL3 | DAP12 |
| CD83 | BTNL3 | BTNL3 | CD32 |
| CD83 | BTNL3 | BTNL3 | CD79a |
| CD83 | BTNL3 | BTNL3 | CD79b |
| CD83 | BTNL3 | NKG2D | CD8 |
| CD83 | BTNL3 | NKG2D | CD3ζ |
| CD83 | BTNL3 | NKG2D | CD3δ |
| CD83 | BTNL3 | NKG2D | CD3γ |
| CD83 | BTNL3 | NKG2D | CD3ε |
| CD83 | BTNL3 | NKG2D | FcγRI-γ |
| CD83 | BTNL3 | NKG2D | FcγRIII-γ |
| CD83 | BTNL3 | NKG2D | FcεRIβ |
| CD83 | BTNL3 | NKG2D | FcεRIγ |
| CD83 | BTNL3 | NKG2D | DAP10 |
| CD83 | BTNL3 | NKG2D | DAP12 |
| CD83 | BTNL3 | NKG2D | CD32 |
| CD83 | BTNL3 | NKG2D | CD79a |
| CD83 | BTNL3 | NKG2D | CD79b |
| CD83 | NKG2D | CD28 | CD8 |
| CD83 | NKG2D | CD28 | CD3ζ |
| CD83 | NKG2D | CD28 | CD3δ |
| CD83 | NKG2D | CD28 | CD3γ |
| CD83 | NKG2D | CD28 | CD3ε |
| CD83 | NKG2D | CD28 | FcγRI-γ |
| CD83 | NKG2D | CD28 | FcγRIII-γ |
| CD83 | NKG2D | CD28 | FcεRIβ |
| CD83 | NKG2D | CD28 | FcεRIγ |
| CD83 | NKG2D | CD28 | DAP10 |
| CD83 | NKG2D | CD28 | DAP12 |
| CD83 | NKG2D | CD28 | CD32 |
| CD83 | NKG2D | CD28 | CD79a |
| CD83 | NKG2D | CD28 | CD79b |
| CD83 | NKG2D | CD8 | CD8 |
| CD83 | NKG2D | CD8 | CD3ζ |
| CD83 | NKG2D | CD8 | CD3δ |
| CD83 | NKG2D | CD8 | CD3γ |
| CD83 | NKG2D | CD8 | CD3ε |
| CD83 | NKG2D | CD8 | FcγRI-γ |
| CD83 | NKG2D | CD8 | FcγRIII-γ |
| CD83 | NKG2D | CD8 | FcεRIβ |
| CD83 | NKG2D | CD8 | FcεRIγ |
| CD83 | NKG2D | CD8 | DAP10 |
| CD83 | NKG2D | CD8 | DAP12 |
| CD83 | NKG2D | CD8 | CD32 |
| CD83 | NKG2D | CD8 | CD79a |
| CD83 | NKG2D | CD8 | CD79b |
| CD83 | NKG2D | CD4 | CD8 |
| CD83 | NKG2D | CD4 | CD3ζ |
| CD83 | NKG2D | CD4 | CD3δ |
| CD83 | NKG2D | CD4 | CD3γ |
| CD83 | NKG2D | CD4 | CD3ε |
| CD83 | NKG2D | CD4 | FcγRI-γ |
| CD83 | NKG2D | CD4 | FcγRIII-γ |
| CD83 | NKG2D | CD4 | FcεRIβ |
| CD83 | NKG2D | CD4 | FcεRIγ |
| CD83 | NKG2D | CD4 | DAP10 |
| CD83 | NKG2D | CD4 | DAP12 |
| CD83 | NKG2D | CD4 | CD32 |
| CD83 | NKG2D | CD4 | CD79a |
| CD83 | NKG2D | CD4 | CD79b |
| CD83 | NKG2D | b2c | CD8 |
| CD83 | NKG2D | b2c | CD3ζ |
| CD83 | NKG2D | b2c | CD3δ |
| CD83 | NKG2D | b2c | CD3γ |
| CD83 | NKG2D | b2c | CD3ε |
| CD83 | NKG2D | b2c | FcγRI-γ |
| CD83 | NKG2D | b2c | FcγRIII-γ |
| CD83 | NKG2D | b2c | FcεRIβ |
| CD83 | NKG2D | b2c | FcεRIγ |
| CD83 | NKG2D | b2c | DAP10 |
| CD83 | NKG2D | b2c | DAP12 |
| CD83 | NKG2D | b2c | CD32 |
| CD83 | NKG2D | b2c | CD79a |
| CD83 | NKG2D | b2c | CD79b |
| CD83 | NKG2D | CD137/41BB | CD8 |
| CD83 | NKG2D | CD137/41BB | CD3ζ |
| CD83 | NKG2D | CD137/41BB | CD3δ |
| CD83 | NKG2D | CD137/41BB | CD3γ |
| CD83 | NKG2D | CD137/41BB | CD3ε |
| CD83 | NKG2D | CD137/41BB | FcγRI-γ |
| CD83 | NKG2D | CD137/41BB | FcγRIII-γ |
| CD83 | NKG2D | CD137/41BB | FcεRIβ |
| CD83 | NKG2D | CD137/41BB | FcεRIγ |
| CD83 | NKG2D | CD137/41BB | DAP10 |
| CD83 | NKG2D | CD137/41BB | DAP12 |
| CD83 | NKG2D | CD137/41BB | CD32 |
| CD83 | NKG2D | CD137/41BB | CD79a |
| CD83 | NKG2D | CD137/41BB | CD79b |
| CD83 | NKG2D | ICOS | CD8 |
| CD83 | NKG2D | ICOS | CD3ζ |
| CD83 | NKG2D | ICOS | CD3δ |
| CD83 | NKG2D | ICOS | CD3γ |
| CD83 | NKG2D | ICOS | CD3ε |
| CD83 | NKG2D | ICOS | FcγRI-γ |
| CD83 | NKG2D | ICOS | FcγRIII-γ |
| CD83 | NKG2D | ICOS | FcεRIβ |
| CD83 | NKG2D | ICOS | FcεRIγ |
| CD83 | NKG2D | ICOS | DAP10 |
| CD83 | NKG2D | ICOS | DAP12 |
| CD83 | NKG2D | ICOS | CD32 |
| CD83 | NKG2D | ICOS | CD79a |
| CD83 | NKG2D | ICOS | CD79b |
| CD83 | NKG2D | CD27 | CD8 |
| CD83 | NKG2D | CD27 | CD3ζ |
| CD83 | NKG2D | CD27 | CD3δ |
| CD83 | NKG2D | CD27 | CD3γ |
| CD83 | NKG2D | CD27 | CD3ε |
| CD83 | NKG2D | CD27 | FcγRI-γ |
| CD83 | NKG2D | CD27 | FcγRIII-γ |
| CD83 | NKG2D | CD27 | FcεRIβ |
| CD83 | NKG2D | CD27 | FcεRIγ |
| CD83 | NKG2D | CD27 | DAP10 |
| CD83 | NKG2D | CD27 | DAP12 |
| CD83 | NKG2D | CD27 | CD32 |
| CD83 | NKG2D | CD27 | CD79a |
| CD83 | NKG2D | CD27 | CD79b |
| CD83 | NKG2D | CD28δ | CD8 |
| CD83 | NKG2D | CD28δ | CD3ζ |
| CD83 | NKG2D | CD28δ | CD3δ |
| CD83 | NKG2D | CD28δ | CD3γ |
| CD83 | NKG2D | CD28δ | CD3ε |
| CD83 | NKG2D | CD28δ | FcγRI-γ |
| CD83 | NKG2D | CD28δ | FcγRIII-γ |
| CD83 | NKG2D | CD28δ | FcεRIβ |
| CD83 | NKG2D | CD28δ | FcεRIγ |
| CD83 | NKG2D | CD28δ | DAP10 |
| CD83 | NKG2D | CD28δ | DAP12 |
| CD83 | NKG2D | CD28δ | CD32 |
| CD83 | NKG2D | CD28δ | CD79a |
| CD83 | NKG2D | CD28δ | CD79b |
| CD83 | NKG2D | CD80 | CD8 |
| CD83 | NKG2D | CD80 | CD3ζ |
| CD83 | NKG2D | CD80 | CD3δ |
| CD83 | NKG2D | CD80 | CD3γ |
| CD83 | NKG2D | CD80 | CD3ε |
| CD83 | NKG2D | CD80 | FcγRI-γ |
| CD83 | NKG2D | CD80 | FcγRIII-γ |
| CD83 | NKG2D | CD80 | FcεRIβ |
| CD83 | NKG2D | CD80 | FcεRIγ |
| CD83 | NKG2D | CD80 | DAP10 |
| CD83 | NKG2D | CD80 | DAP12 |
| CD83 | NKG2D | CD80 | CD32 |
| CD83 | NKG2D | CD80 | CD79a |
| CD83 | NKG2D | CD80 | CD79b |
| CD83 | NKG2D | CD86 | CD8 |
| CD83 | NKG2D | CD86 | CD3ζ |
| CD83 | NKG2D | CD86 | CD3δ |
| CD83 | NKG2D | CD86 | CD3γ |
| CD83 | NKG2D | CD86 | CD3ε |
| CD83 | NKG2D | CD86 | FcγRI-γ |
| CD83 | NKG2D | CD86 | FcγRIII-γ |
| CD83 | NKG2D | CD86 | FcεRIβ |
| CD83 | NKG2D | CD86 | FcεRIγ |
| CD83 | NKG2D | CD86 | DAP10 |
| CD83 | NKG2D | CD86 | DAP12 |
| CD83 | NKG2D | CD86 | CD32 |
| CD83 | NKG2D | CD86 | CD79a |
| CD83 | NKG2D | CD86 | CD79b |
| CD83 | NKG2D | OX40 | CD8 |
| CD83 | NKG2D | OX40 | CD3ζ |
| CD83 | NKG2D | OX40 | CD3δ |
| CD83 | NKG2D | OX40 | CD3γ |
| CD83 | NKG2D | OX40 | CD3ε |
| CD83 | NKG2D | OX40 | FcγRI-γ |
| CD83 | NKG2D | OX40 | FcγRIII-γ |
| CD83 | NKG2D | OX40 | FcεRIβ |
| CD83 | NKG2D | OX40 | FcεRIγ |
| CD83 | NKG2D | OX40 | DAP10 |
| CD83 | NKG2D | OX40 | DAP12 |
| CD83 | NKG2D | OX40 | CD32 |
| CD83 | NKG2D | OX40 | CD79a |
| CD83 | NKG2D | OX40 | CD79b |
| CD83 | NKG2D | DAP10 | CD8 |
| CD83 | NKG2D | DAP10 | CD3ζ |
| CD83 | NKG2D | DAP10 | CD3δ |
| CD83 | NKG2D | DAP10 | CD3γ |
| CD83 | NKG2D | DAP10 | CD3ε |
| CD83 | NKG2D | DAP10 | FcγRI-γ |
| CD83 | NKG2D | DAP10 | FcγRIII-γ |
| CD83 | NKG2D | DAP10 | FcεRIβ |
| CD83 | NKG2D | DAP10 | FcεRIγ |
| CD83 | NKG2D | DAP10 | DAP10 |
| CD83 | NKG2D | DAP10 | DAP12 |
| CD83 | NKG2D | DAP10 | CD32 |
| CD83 | NKG2D | DAP10 | CD79a |
| CD83 | NKG2D | DAP10 | CD79b |
| CD83 | NKG2D | DAP12 | CD8 |
| CD83 | NKG2D | DAP12 | CD3ζ |
| CD83 | NKG2D | DAP12 | CD3δ |
| CD83 | NKG2D | DAP12 | CD3γ |
| CD83 | NKG2D | DAP12 | CD3ε |
| CD83 | NKG2D | DAP12 | FcγRI-γ |
| CD83 | NKG2D | DAP12 | FcγRIII-γ |
| CD83 | NKG2D | DAP12 | FcεRIβ |
| CD83 | NKG2D | DAP12 | FcεRIγ |
| CD83 | NKG2D | DAP12 | DAP10 |
| CD83 | NKG2D | DAP12 | DAP12 |
| CD83 | NKG2D | DAP12 | CD32 |
| CD83 | NKG2D | DAP12 | CD79a |
| CD83 | NKG2D | DAP12 | CD79b |
| CD83 | NKG2D | MyD88 | CD8 |
| CD83 | NKG2D | MyD88 | CD3ζ |
| CD83 | NKG2D | MyD88 | CD3δ |
| CD83 | NKG2D | MyD88 | CD3γ |
| CD83 | NKG2D | MyD88 | CD3ε |
| CD83 | NKG2D | MyD88 | FcγRI-γ |
| CD83 | NKG2D | MyD88 | FcγRIII-γ |
| CD83 | NKG2D | MyD88 | FcεRIβ |
| CD83 | NKG2D | MyD88 | FcεRIγ |
| CD83 | NKG2D | MyD88 | DAP10 |
| CD83 | NKG2D | MyD88 | DAP12 |
| CD83 | NKG2D | MyD88 | CD32 |
| CD83 | NKG2D | MyD88 | CD79a |
| CD83 | NKG2D | MyD88 | CD79b |
| CD83 | NKG2D | CD7 | CD8 |
| CD83 | NKG2D | CD7 | CD3ζ |
| CD83 | NKG2D | CD7 | CD3δ |
| CD83 | NKG2D | CD7 | CD3γ |
| CD83 | NKG2D | CD7 | CD3ε |
| CD83 | NKG2D | CD7 | FcγRI-γ |
| CD83 | NKG2D | CD7 | FcγRIII-γ |
| CD83 | NKG2D | CD7 | FcεRIβ |
| CD83 | NKG2D | CD7 | FcεRIγ |
| CD83 | NKG2D | CD7 | DAP10 |
| CD83 | NKG2D | CD7 | DAP12 |
| CD83 | NKG2D | CD7 | CD32 |
| CD83 | NKG2D | CD7 | CD79a |
| CD83 | NKG2D | CD7 | CD79b |
| CD83 | NKG2D | BTNL3 | CD8 |
| CD83 | NKG2D | BTNL3 | CD3ζ |
| CD83 | NKG2D | BTNL3 | CD3δ |
| CD83 | NKG2D | BTNL3 | CD3γ |
| CD83 | NKG2D | BTNL3 | CD3ε |
| CD83 | NKG2D | BTNL3 | FcγRI-γ |
| CD83 | NKG2D | BTNL3 | FcγRIII-γ |
| CD83 | NKG2D | BTNL3 | FcεRIβ |
| CD83 | NKG2D | BTNL3 | FcεRIγ |
| CD83 | NKG2D | BTNL3 | DAP10 |
| CD83 | NKG2D | BTNL3 | DAP12 |
| CD83 | NKG2D | BTNL3 | CD32 |
| CD83 | NKG2D | BTNL3 | CD79a |
| CD83 | NKG2D | BTNL3 | CD79b |
| CD83 | NKG2D | NKG2D | CD8 |
| CD83 | NKG2D | NKG2D | CD3ζ |
| CD83 | NKG2D | NKG2D | CD3δ |
| CD83 | NKG2D | NKG2D | CD3γ |
| CD83 | NKG2D | NKG2D | CD3ε |
| CD83 | NKG2D | NKG2D | FcγRI-γ |
| CD83 | NKG2D | NKG2D | FcγRIII-γ |
| CD83 | NKG2D | NKG2D | FcεRIβ |
| CD83 | NKG2D | NKG2D | FcεRIγ |
| CD83 | NKG2D | NKG2D | DAP10 |
| CD83 | NKG2D | NKG2D | DAP12 |
| CD83 | NKG2D | NKG2D | CD32 |
| CD83 | NKG2D | NKG2D | CD79a |
| CD83 | NKG2D | NKG2D | CD79b |

Tables 4, 5, and 6 below provide some example combinations of CD83 or CD19-binding region, co-stimulatory signaling regions, and intracellular signaling domain that can occur in in the dual CAR systems.

TABLE 4

CARs lacking Co-Stimulatory Signal (for dual CAR approach)

| | | |
|---|---|---|
| ScFv | Co-stimulatory Signal | Signal Domain |
| CD83 or CD19 | none | CD8 |
| CD83 or CD19 | none | CD3ζ |
| CD83 or CD19 | none | CD3δ |
| CD83 or CD19 | none | CD3γ |
| CD83 or CD19 | none | CD3ε |
| CD83 or CD19 | none | FcγRI-γ |
| CD83 or CD19 | none | FcγRIII-γ |
| CD83 or CD19 | none | FcεRIβ |
| CD83 or CD19 | none | FcεRIγ |
| CD83 or CD19 | none | DAP10 |
| CD83 or CD19 | none | DAP12 |
| CD83 or CD19 | none | CD32 |
| CD83 or CD19 | none | CD79a |
| CD83 or CD19 | none | CD8 |
| CD83 or CD19 | none | CD3ζ |
| CD83 or CD19 | none | CD3δ |
| CD83 or CD19 | none | CD3γ |
| CD83 or CD19 | none | CD3ε |
| CD83 or CD19 | none | FcγRI-γ |

TABLE 5

CARs lacking Signal Domain (for dual CAR approach)

| | | |
|---|---|---|
| ScFv | Co-stimulatory Signal | Signal Domain |
| CD83 or CD19 | CD28 | none |
| CD83 or CD19 | CD8 | none |
| CD83 or CD19 | CD4 | none |
| CD83 or CD19 | b2c | none |
| CD83 or CD19 | CD137/41BB | none |
| CD83 or CD19 | ICOS | none |
| CD83 or CD19 | CD27 | none |
| CD83 or CD19 | CD28δ | none |
| CD83 or CD19 | CD80 | none |
| CD83 or CD19 | CD86 | none |
| CD83 or CD19 | OX40 | none |
| CD83 or CD19 | DAP10 | none |
| CD83 or CD19 | MyD88 | none |
| CD83 or CD19 | CD7 | none |
| CD83 or CD19 | DAP12 | none |
| CD83 or CD19 | MyD88 | none |
| CD83 or CD19 | CD7 | none |
| CD83 or CD19 | BTNL3 | none |
| CD83 or CD19 | NKG2D | none |

TABLE 6

Third Generation CARs Lacking Signal Domain (for dual CAR Approach)

| | | | |
|---|---|---|---|
| ScFv | Co-Stimulatory Signal | Co-Stimulatory Signal | Signal Domain |
| CD83 or CD19 | CD28 | CD28 | none |
| CD83 or CD19 | CD28 | CD8 | none |
| CD83 or CD19 | CD28 | CD4 | none |
| CD83 or CD19 | CD28 | b2c | none |
| CD83 or CD19 | CD28 | CD137/41BB | none |
| CD83 or CD19 | CD28 | ICOS | none |
| CD83 or CD19 | CD28 | CD27 | none |
| CD83 or CD19 | CD28 | CD28δ | none |
| CD83 or CD19 | CD28 | CD80 | none |
| CD83 or CD19 | CD28 | CD86 | none |
| CD83 or CD19 | CD28 | OX40 | none |
| CD83 or CD19 | CD28 | DAP10 | none |
| CD83 or CD19 | CD28 | MyD88 | none |
| CD83 or CD19 | CD28 | CD7 | none |
| CD83 or CD19 | CD28 | DAP12 | none |
| CD83 or CD19 | CD28 | MyD88 | none |
| CD83 or CD19 | CD28 | CD7 | none |
| CD83 or CD19 | CD8 | CD28 | none |
| CD83 or CD19 | CD8 | CD8 | none |
| CD83 or CD19 | CD8 | CD4 | none |
| CD83 or CD19 | CD8 | b2c | none |
| CD83 or CD19 | CD8 | CD137/41BB | none |
| CD83 or CD19 | CD8 | ICOS | none |
| CD83 or CD19 | CD8 | CD27 | none |
| CD83 or CD19 | CD8 | CD28δ | none |
| CD83 or CD19 | CD8 | CD80 | none |
| CD83 or CD19 | CD8 | CD86 | none |
| CD83 or CD19 | CD8 | OX40 | none |
| CD83 or CD19 | CD8 | DAP10 | none |
| CD83 or CD19 | CD8 | MyD88 | none |
| CD83 or CD19 | CD8 | CD7 | none |
| CD83 or CD19 | CD8 | DAP12 | none |
| CD83 or CD19 | CD8 | MyD88 | none |
| CD83 or CD19 | CD8 | CD7 | none |
| CD83 or CD19 | CD4 | CD28 | none |
| CD83 or CD19 | CD4 | CD8 | none |
| CD83 or CD19 | CD4 | CD4 | none |
| CD83 or CD19 | CD4 | b2c | none |
| CD83 or CD19 | CD4 | CD137/41BB | none |
| CD83 or CD19 | CD4 | ICOS | none |
| CD83 or CD19 | CD4 | CD27 | none |
| CD83 or CD19 | CD4 | CD28δ | none |
| CD83 or CD19 | CD4 | CD80 | none |
| CD83 or CD19 | CD4 | CD86 | none |
| CD83 or CD19 | CD4 | OX40 | none |
| CD83 or CD19 | CD4 | DAP10 | none |
| CD83 or CD19 | CD4 | MyD88 | none |
| CD83 or CD19 | CD4 | CD7 | none |
| CD83 or CD19 | CD4 | DAP12 | none |
| CD83 or CD19 | CD4 | MyD88 | none |
| CD83 or CD19 | CD4 | CD7 | none |
| CD83 or CD19 | b2c | CD28 | none |
| CD83 or CD19 | b2c | CD8 | none |
| CD83 or CD19 | b2c | CD4 | none |
| CD83 or CD19 | b2c | b2c | none |
| CD83 or CD19 | b2c | CD137/41BB | none |
| CD83 or CD19 | b2c | ICOS | none |
| CD83 or CD19 | b2c | CD27 | none |
| CD83 or CD19 | b2c | CD28δ | none |
| CD83 or CD19 | b2c | CD80 | none |
| CD83 or CD19 | b2c | CD86 | none |
| CD83 or CD19 | b2c | OX40 | none |
| CD83 or CD19 | b2c | DAP10 | none |
| CD83 or CD19 | b2c | MyD88 | none |
| CD83 or CD19 | b2c | CD7 | none |
| CD83 or CD19 | b2c | DAP12 | none |
| CD83 or CD19 | b2c | MyD88 | none |
| CD83 or CD19 | b2c | CD7 | none |
| CD83 or CD19 | CD137/41BB | CD28 | none |
| CD83 or CD19 | CD137/41BB | CD8 | none |
| CD83 or CD19 | CD137/41BB | CD4 | none |
| CD83 or CD19 | CD137/41BB | b2c | none |
| CD83 or CD19 | CD137/41BB | CD137/41BB | none |
| CD83 or CD19 | CD137/41BB | ICOS | none |
| CD83 or CD19 | CD137/41BB | CD27 | none |
| CD83 or CD19 | CD137/41BB | CD28δ | none |
| CD83 or CD19 | CD137/41BB | CD80 | none |
| CD83 or CD19 | CD137/41BB | CD86 | none |
| CD83 or CD19 | CD137/41BB | OX40 | none |
| CD83 or CD19 | CD137/41BB | DAP10 | none |
| CD83 or CD19 | CD137/41BB | MyD88 | none |
| CD83 or CD19 | CD137/41BB | CD7 | none |
| CD83 or CD19 | CD137/41BB | DAP12 | none |
| CD83 or CD19 | CD137/41BB | MyD88 | none |
| CD83 or CD19 | CD137/41BB | CD7 | none |
| CD83 or CD19 | ICOS | CD28 | none |
| CD83 or CD19 | ICOS | CD8 | none |
| CD83 or CD19 | ICOS | CD4 | none |
| CD83 or CD19 | ICOS | b2c | none |
| CD83 or CD19 | ICOS | CD137/41BB | none |
| CD83 or CD19 | ICOS | ICOS | none |
| CD83 or CD19 | ICOS | CD27 | none |
| CD83 or CD19 | ICOS | CD28δ | none |
| CD83 or CD19 | ICOS | CD80 | none |
| CD83 or CD19 | ICOS | CD86 | none |
| CD83 or CD19 | ICOS | OX40 | none |
| CD83 or CD19 | ICOS | DAP10 | none |
| CD83 or CD19 | ICOS | MyD88 | none |
| CD83 or CD19 | ICOS | CD7 | none |
| CD83 or CD19 | ICOS | DAP12 | none |
| CD83 or CD19 | ICOS | MyD88 | none |
| CD83 or CD19 | ICOS | CD7 | none |
| CD83 or CD19 | ICOS | CD28 | none |
| CD83 or CD19 | ICOS | CD8 | none |
| CD83 or CD19 | ICOS | CD4 | none |
| CD83 or CD19 | ICOS | b2c | none |
| CD83 or CD19 | ICOS | CD137/41BB | none |
| CD83 or CD19 | ICOS | ICOS | none |
| CD83 or CD19 | ICOS | CD27 | none |
| CD83 or CD19 | ICOS | CD28δ | none |
| CD83 or CD19 | ICOS | CD80 | none |
| CD83 or CD19 | ICOS | CD86 | none |
| CD83 or CD19 | ICOS | OX40 | none |
| CD83 or CD19 | ICOS | DAP10 | none |
| CD83 or CD19 | ICOS | MyD88 | none |
| CD83 or CD19 | ICOS | CD7 | none |
| CD83 or CD19 | ICOS | DAP12 | none |
| CD83 or CD19 | ICOS | MyD88 | none |
| CD83 or CD19 | ICOS | CD7 | none |
| CD83 or CD19 | CD27 | CD28 | none |
| CD83 or CD19 | CD27 | CD8 | none |
| CD83 or CD19 | CD27 | CD4 | none |
| CD83 or CD19 | CD27 | b2c | none |
| CD83 or CD19 | CD27 | CD137/41BB | none |
| CD83 or CD19 | CD27 | ICOS | none |
| CD83 or CD19 | CD27 | CD27 | none |
| CD83 or CD19 | CD27 | CD28δ | none |
| CD83 or CD19 | CD27 | CD80 | none |
| CD83 or CD19 | CD27 | CD86 | none |
| CD83 or CD19 | CD27 | OX40 | none |
| CD83 or CD19 | CD27 | DAP10 | none |
| CD83 or CD19 | CD27 | MyD88 | none |
| CD83 or CD19 | CD27 | CD7 | none |
| CD83 or CD19 | CD27 | DAP12 | none |
| CD83 or CD19 | CD27 | MyD88 | none |
| CD83 or CD19 | CD27 | CD7 | none |
| CD83 or CD19 | CD28δ | CD28 | none |
| CD83 or CD19 | CD28δ | CD8 | none |
| CD83 or CD19 | CD28δ | CD4 | none |
| CD83 or CD19 | CD28δ | b2c | none |
| CD83 or CD19 | CD28δ | CD137/41BB | none |
| CD83 or CD19 | CD28δ | ICOS | none |
| CD83 or CD19 | CD28δ | CD27 | none |
| CD83 or CD19 | CD28δ | CD28δ | none |
| CD83 or CD19 | CD28δ | CD80 | none |
| CD83 or CD19 | CD28δ | CD86 | none |
| CD83 or CD19 | CD28δ | OX40 | none |
| CD83 or CD19 | CD28δ | DAP10 | none |
| CD83 or CD19 | CD28δ | MyD88 | none |
| CD83 or CD19 | CD28δ | CD7 | none |
| CD83 or CD19 | CD28δ | DAP12 | none |
| CD83 or CD19 | CD28δ | MyD88 | none |
| CD83 or CD19 | CD28δ | CD7 | none |
| CD83 or CD19 | CD80 | CD28 | none |
| CD83 or CD19 | CD80 | CD8 | none |
| CD83 or CD19 | CD80 | CD4 | none |
| CD83 or CD19 | CD80 | b2c | none |
| CD83 or CD19 | CD80 | CD137/41BB | none |
| CD83 or CD19 | CD80 | ICOS | none |
| CD83 or CD19 | CD80 | CD27 | none |
| CD83 or CD19 | CD80 | CD28δ | none |
| CD83 or CD19 | CD80 | CD80 | none |
| CD83 or CD19 | CD80 | CD86 | none |
| CD83 or CD19 | CD80 | OX40 | none |
| CD83 or CD19 | CD80 | DAP10 | none |
| CD83 or CD19 | CD80 | MyD88 | none |
| CD83 or CD19 | CD80 | CD7 | none |
| CD83 or CD19 | CD80 | DAP12 | none |
| CD83 or CD19 | CD80 | MyD88 | none |
| CD83 or CD19 | CD80 | CD7 | none |
| CD83 or CD19 | CD86 | CD28 | none |
| CD83 or CD19 | CD86 | CD8 | none |
| CD83 or CD19 | CD86 | CD4 | none |
| CD83 or CD19 | CD86 | b2c | none |
| CD83 or CD19 | CD86 | CD137/41BB | none |
| CD83 or CD19 | CD86 | ICOS | none |
| CD83 or CD19 | CD86 | CD27 | none |
| CD83 or CD19 | CD86 | CD28δ | none |
| CD83 or CD19 | CD86 | CD80 | none |
| CD83 or CD19 | CD86 | CD86 | none |
| CD83 or CD19 | CD86 | OX40 | none |
| CD83 or CD19 | CD86 | DAP10 | none |
| CD83 or CD19 | CD86 | MyD88 | none |
| CD83 or CD19 | CD86 | CD7 | none |
| CD83 or CD19 | CD86 | DAP12 | none |
| CD83 or CD19 | CD86 | MyD88 | none |
| CD83 or CD19 | CD86 | CD7 | none |
| CD83 or CD19 | OX40 | CD28 | none |
| CD83 or CD19 | OX40 | CD8 | none |
| CD83 or CD19 | OX40 | CD4 | none |
| CD83 or CD19 | OX40 | b2c | none |
| CD83 or CD19 | OX40 | CD137/41BB | none |
| CD83 or CD19 | OX40 | ICOS | none |
| CD83 or CD19 | OX40 | CD27 | none |
| CD83 or CD19 | OX40 | CD28δ | none |
| CD83 or CD19 | OX40 | CD80 | none |
| CD83 or CD19 | OX40 | CD86 | none |
| CD83 or CD19 | OX40 | OX40 | none |
| CD83 or CD19 | OX40 | DAP10 | none |
| CD83 or CD19 | OX40 | MyD88 | none |
| CD83 or CD19 | OX40 | CD7 | none |
| CD83 or CD19 | OX40 | DAP12 | none |
| CD83 or CD19 | OX40 | MyD88 | none |
| CD83 or CD19 | OX40 | CD7 | none |
| CD83 or CD19 | DAP10 | CD28 | none |
| CD83 or CD19 | DAP10 | CD8 | none |
| CD83 or CD19 | DAP10 | CD4 | none |
| CD83 or CD19 | DAP10 | b2c | none |
| CD83 or CD19 | DAP10 | CD137/41BB | none |
| CD83 or CD19 | DAP10 | ICOS | none |
| CD83 or CD19 | DAP10 | CD27 | none |
| CD83 or CD19 | DAP10 | CD28δ | none |
| CD83 or CD19 | DAP10 | CD80 | none |
| CD83 or CD19 | DAP10 | CD86 | none |
| CD83 or CD19 | DAP10 | OX40 | none |
| CD83 or CD19 | DAP10 | DAP10 | none |
| CD83 or CD19 | DAP10 | MyD88 | none |
| CD83 or CD19 | DAP10 | CD7 | none |
| CD83 or CD19 | DAP10 | DAP12 | none |
| CD83 or CD19 | DAP10 | MyD88 | none |
| CD83 or CD19 | DAP10 | CD7 | none |
| CD83 or CD19 | DAP12 | CD28 | none |
| CD83 or CD19 | DAP12 | CD8 | none |
| CD83 or CD19 | DAP12 | CD4 | none |
| CD83 or CD19 | DAP12 | b2c | none |
| CD83 or CD19 | DAP12 | CD137/41BB | none |
| CD83 or CD19 | DAP12 | ICOS | none |
| CD83 or CD19 | DAP12 | CD27 | none |
| CD83 or CD19 | DAP12 | CD28δ | none |
| CD83 or CD19 | DAP12 | CD80 | none |
| CD83 or CD19 | DAP12 | CD86 | none |
| CD83 or CD19 | DAP12 | OX40 | none |
| CD83 or CD19 | DAP12 | DAP10 | none |
| CD83 or CD19 | DAP12 | MyD88 | none |
| CD83 or CD19 | DAP12 | CD7 | none |
| CD83 or CD19 | DAP12 | DAP12 | none |
| CD83 or CD19 | DAP12 | MyD88 | none |
| CD83 or CD19 | DAP12 | CD7 | none |
| CD83 or CD19 | MyD88 | CD28 | none |
| CD83 or CD19 | MyD88 | CD8 | none |
| CD83 or CD19 | MyD88 | CD4 | none |
| CD83 or CD19 | MyD88 | b2c | none |
| CD83 or CD19 | MyD88 | CD137/41BB | none |
| CD83 or CD19 | MyD88 | ICOS | none |
| CD83 or CD19 | MyD88 | CD27 | none |
| CD83 or CD19 | MyD88 | CD28δ | none |
| CD83 or CD19 | MyD88 | CD80 | none |
| CD83 or CD19 | MyD88 | CD86 | none |
| CD83 or CD19 | MyD88 | OX40 | none |
| CD83 or CD19 | MyD88 | DAP10 | none |
| CD83 or CD19 | MyD88 | MyD88 | none |
| CD83 or CD19 | MyD88 | CD7 | none |
| CD83 or CD19 | MyD88 | DAP12 | none |
| CD83 or CD19 | MyD88 | MyD88 | none |
| CD83 or CD19 | MyD88 | CD7 | none |
| CD83 or CD19 | CD7 | CD28 | none |
| CD83 or CD19 | CD7 | CD8 | none |
| CD83 or CD19 | CD7 | CD4 | none |
| CD83 or CD19 | CD7 | b2c | none |
| CD83 or CD19 | CD7 | CD137/41BB | none |
| CD83 or CD19 | CD7 | ICOS | none |
| CD83 or CD19 | CD7 | CD27 | none |
| CD83 or CD19 | CD7 | CD28δ | none |
| CD83 or CD19 | CD7 | CD80 | none |
| CD83 or CD19 | CD7 | CD86 | none |
| CD83 or CD19 | CD7 | OX40 | none |
| CD83 or CD19 | CD7 | DAP10 | none |
| CD83 or CD19 | CD7 | MyD88 | none |
| CD83 or CD19 | CD7 | CD7 | none |
| CD83 or CD19 | CD7 | DAP12 | none |
| CD83 or CD19 | CD7 | MyD88 | none |
| CD83 or CD19 | CD7 | CD7 | none |
| CD83 or CD19 | BTNL3 | CD28 | none |
| CD83 or CD19 | BTNL3 | CD8 | none |
| CD83 or CD19 | BTNL3 | CD4 | none |
| CD83 or CD19 | BTNL3 | b2c | none |
| CD83 or CD19 | BTNL3 | CD137/41BB | none |
| CD83 or CD19 | BTNL3 | ICOS | none |
| CD83 or CD19 | BTNL3 | CD27 | none |
| CD83 or CD19 | BTNL3 | CD28δ | none |
| CD83 or CD19 | BTNL3 | CD80 | none |
| CD83 or CD19 | BTNL3 | CD86 | none |
| CD83 or CD19 | BTNL3 | OX40 | none |
| CD83 or CD19 | BTNL3 | DAP10 | none |
| CD83 or CD19 | BTNL3 | MyD88 | none |
| CD83 or CD19 | BTNL3 | CD7 | none |
| CD83 or CD19 | BTNL3 | DAP12 | none |
| CD83 or CD19 | BTNL3 | MyD88 | none |
| CD83 or CD19 | BTNL3 | CD7 | none |
| CD83 or CD19 | NKG2D | CD28 | none |
| CD83 or CD19 | NKG2D | CD8 | none |
| CD83 or CD19 | NKG2D | CD4 | none |
| CD83 or CD19 | NKG2D | b2c | none |
| CD83 or CD19 | NKG2D | CD137/41BB | none |
| CD83 or CD19 | NKG2D | ICOS | none |
| CD83 or CD19 | NKG2D | CD27 | none |
| CD83 or CD19 | NKG2D | CD28δ | none |
| CD83 or CD19 | NKG2D | CD80 | none |
| CD83 or CD19 | NKG2D | CD86 | none |
| CD83 or CD19 | NKG2D | OX40 | none |
| CD83 or CD19 | NKG2D | DAP10 | none |
| CD83 or CD19 | NKG2D | MyD88 | none |
| CD83 or CD19 | NKG2D | CD7 | none |
| CD83 or CD19 | NKG2D | DAP12 | none |
| CD83 or CD19 | NKG2D | MyD88 | none |
| CD83 or CD19 | NKG2D | CD7 | none |

In some embodiments, the anti-CD83 and/or anti-CD19 binding agent is single chain variable fragment (scFv) antibody. The affinity/specificity of an scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some embodiments, the binding agent is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

Also disclosed are bi-specific CARs that target CD83 and CD19. For example, in these embodiments, the endodomain of the disclosed CAR can contain only a signaling domain (SD) or a co-stimulatory signaling region (CSR), but not both. The second CAR provides the missing signal if it is activated. For example, if an anti-CD83 CAR contains an SD but not a CSR, then the immune cell containing this CAR is only activated if the anti-CD19 CAR containing a CSR binds its respective antigen. Likewise, if the anti-CD83 CAR contains a CSR but not a SD, then the immune cell containing this CAR is only activated if the anti-CD19 CAR containing an SD binds its respective antigen.

### Nucleic Acids and Vectors

Also disclosed are polynucleotides and polynucleotide vectors encoding the disclosed CARs that allow expression of the CARs in the disclosed immune cells.

Nucleic acid sequences encoding the disclosed CARs, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

Expression of nucleic acids encoding CARs is typically achieved by operably linking a nucleic acid encoding the CAR polypeptide to a promoter, and incorporating the construct into an expression vector. Typical cloning vectors contain transcription and translation terminators, initiation sequences, and promoters useful for regulation of the expression of the desired nucleic acid sequence.

The disclosed nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. In some embodiments, the polynucleotide vectors are lentiviral or retroviral vectors.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, MND (myeloproliferative sarcoma virus) promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. The promoter can alternatively be an inducible promoter. Examples of inducible promoters include, but are not limited to a metallothionine promoter, a glucocorticoid promoter, a progesterone promoter, and a tetracycline promoter.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma, St Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc, (Birmingham, Ala.).

### Genetically Engineered Immune Cells Expressing CAR Polypeptides

Also disclosed are immune cells (a.k.a., immune effector cells) that are engineered to express one or more of the disclosed CARs (also referred to herein as “CAR-T cells”). These cells are preferably obtained from the subject to be treated (i.e. are autologous). However, in some embodiments, immune cell lines or donor immune cells (allogeneic) can be used. Immune cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune cells can be obtained from blood collected from a subject using any number of techniques known to the skilled artisan, such as Ficoll™ separation. For example, cells from the circulating blood of an individual may be obtained by apheresis. In some embodiments, immune cells are isolated from peripheral blood lymphocytes by lysing the red blood cells and depleting the monocytes, for example, by centrifugation through a PERCOLL™ gradient or by counterflow centrifugal elutriation. A specific subpopulation of immune cells can be further isolated by positive or negative selection techniques. For example, immune cells can be isolated using a combination of antibodies directed to surface markers unique to the positively selected cells, e.g., by incubation with antibody-conjugated beads for a time period sufficient for positive selection of the desired immune cells. Alternatively, enrichment of one or more immune cells subpopulations can be accomplished by negative selection using a combination of antibodies directed to surface markers unique to the negatively selected cells.

In some embodiments, the genetically engineered immune cells disclosed herein may comprise any leukocyte involved in defending the body against infectious disease and foreign materials. For example, the immune cells can comprise lymphocytes, monocytes, macrophages, dentritic cells, mast cells, neutrophils, basophils, eosinophils, or any combinations thereof. In some examples, the immune cells can comprise T lymphocytes.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (T_{H} cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (T_{C} cells, or CTLs) destroy virally infected cells and tumor cells, and are also implicated in transplant rejection. These cells are also known as CD8⁺ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4⁺ or CD8⁺. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described - naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8⁺ T lymphocytes. In some embodiments, the T cells comprise γδ T cells, which possess a distinct T-cell receptor (TCR) having one γ chain and one δ chain instead of a and β chains.

Natural-killer (NK) cells are CD56⁺CD3⁻ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8⁺ T lymphocytes, NK cells launch cytotoxicity against tumor cells without the requirement for prior sensitization, and can also eradicate MHC-I-negative cells (Narni-Mancinelli E, et al. Int Immunol 2011 23:427-431). NK cells are safer effector cells, as they may avoid the potentially lethal complications of cytokine storms (Morgan RA, et al. Mol Ther 2010 18:843-851), tumor lysis syndrome (Porter DL, et al. N Engl J Med 2011 365:725-733), and on-target, off-tumor effects.

### Therapeutic Methods for Treating Autoimmune Diseases

Any of the immune cells disclosed herein, which are genetically engineered to express one or more of the disclosed CARs (e.g., an anti-CD83 CAR or a combination of an anti-CD83 and CD19 CAR constructs) can be used to suppress and/or kill alloreactive and/or autoreactive cells, such as B-cells, and treat or prevent autoimmune diseases. Therefore, the disclosed genetically engineered immune cells can be administered to a subject in need of the treatment, for example, having or at risk for having an autoimmune disease.

The disclosed CAR-expressing immune cells may be administered either alone, or as a pharmaceutical composition in combination with diluents and/or with other components such as IL-2, IL-15, or other cytokines or cell populations.

In some embodiments, the disclosed CAR-expressing immune cells can be administered in combination with ER stress blockade (compounds to target the IRE-1/XBP-1 pathway (e.g., B-I09). In some embodiments, the disclosed CAR-expressing immune cells can be administered in combination with a JAK2 inhibitor, a STAT3 inhibitor, an Aurora kinase inhibitor, an mTOR inhibitor, or any combination thereof.

Briefly, pharmaceutical compositions may comprise any of the genetically engineered immune cell population as described herein (e.g., expressing an anti-CD83 CAR or a combination of anti-CD83/CD19 CAR polypeptides), in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat MM. The quantity and frequency of administration will be determined by such factors as the condition of the patient, and the severity of the patient’s disease, although appropriate dosages may be determined by clinical trials.

When a “therapeutic amount” is indicated, the precise amount of the compositions of the present invention to be administered can be determined by a physician with consideration of individual differences in age, weight, extent of transplantation, and condition of the patient (subject). It can generally be stated that a pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient subcutaneously, intradermally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a site of transplantation.

In certain embodiments, the disclosed CAR-modified immune cells are administered to a patient in conjunction with (e.g., before, simultaneously or following) any number of relevant treatment modalities, including but not limited to thalidomide, dexamethasone, bortezomib, and lenalidomide. In further embodiments, the CAR-modified immune cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

One primary concern with CAR-T cells as a form of “living therapeutic” is their manipulability in vivo and their potential immune-stimulating side effects. To better control CAR-T therapy and prevent against unwanted side effects, a variety of features have been engineered including off-switches, safety mechanisms, and conditional control mechanisms. Both self-destruct and marked/tagged CAR-T cells for example, are engineered to have an “off-switch” that promotes clearance of the CAR-expressing T-cell. A self-destruct CAR-T contains a CAR, but is also engineered to express a pro-apoptotic suicide gene or “elimination gene” inducible upon administration of an exogenous molecule. A variety of suicide genes may be employed for this purpose, including HSV-TK (herpes simplex virus thymidine kinase), Fas, iCasp9 (inducible caspase 9), CD20, MYC TAG, and truncated EGFR (endothelial growth factor receptor). HSK for example, will convert the prodrug ganciclovir (GCV) into GCV-triphosphate that incorporates itself into replicating DNA, ultimately leading to cell death. iCasp9 is a chimeric protein containing components of FK506-binding protein that binds the small molecule AP1903, leading to caspase 9 dimerization and apoptosis. A marked/ tagged CAR-T cell however, is one that possesses a CAR but also is engineered to express a selection marker. Administration of a mAb against this selection marker will promote clearance of the CAR-T cell. Truncated EGFR is one such targetable antigen by the anti-EGFR mAb, and administration of cetuximab works to promotes elimination of the CAR-T cell. CARs created to have these features are also referred to as sCARs for ‘switchable CARs’, and RCARs for ‘regulatable CARs’. A “safety CAR”, also known as an “inhibitory CAR” (iCAR), is engineered to express two antigen binding domains. One of these extracellular domains is directed against a first antigen and bound to an intracellular costimulatory and stimulatory domain. The second extracellular antigen binding domain however is specific for normal tissue and bound to an intracellular checkpoint domain such as CTLA4, PD1, or CD45. Incorporation of multiple intracellular inhibitory domains to the iCAR is also possible. Some inhibitory molecules that may provide these inhibitory domains include B7-H1, B7-1, CD160, PIH, 2B4, CEACAM (CEACAM-1, CEACAM-3, and/or CEACAM-5), LAG-3, TIGIT, BTLA, LAIR1, and TGFβ-R. In the presence of normal tissue, stimulation of this second antigen binding domain will work to inhibit the CAR. It should be noted that due to this dual antigen specificity, iCARs are also a form of bi-specific CAR-T cells. The safety CAR-T engineering enhances specificity of the CAR-T cell for tissue, and is advantageous in situations where certain normal tissues may express very low levels of a antigen that would lead to off target effects with a standard CAR (Morgan 2010).

A conditional CAR-T cell expresses an extracellular antigen binding domain connected to an intracellular costimulatory domain and a separate, intracellular costimulator. The costimulatory and stimulatory domain sequences are engineered in such a way that upon administration of an exogenous molecule the resultant proteins will come together intracellularly to complete the CAR circuit In this way, CAR-T activation can be modulated, and possibly even ‘fine-tuned’ or personalized to a specific patient. Similar to a dual CAR design, the stimulatory and costimulatory domains are physically separated when inactive in the conditional CAR; for this reason these too are also referred to as a “split CAR”.

Typically, CAR-T cells are created using α-β T cells, however γ-δ T cells may also be used. In some embodiments, the described CAR constructs, domains, and engineered features used to generate CAR-T cells could similarly be employed in the generation of other types of CAR-expressing immune cells including NK (natural killer) cells, B cells, mast cells, myeloid-derived phagocytes, and NKT cells. Alternatively, a CAR-expressing cell may be created to have properties of both T-cell and NK cells. In an additional embodiment, the transduced with CARs may be autologous or allogeneic.

Several different methods for CAR expression may be used including retroviral transduction (including γ-retroviral), lentiviral transduction, transposon/transposases (Sleeping Beauty and PiggyBac systems), and messenger RNA transfer-mediated gene expression. Gene editing (gene insertion or gene deletion/disruption) has become of increasing importance with respect to the possibility for engineering CAR-T cells as well. CRISPR-Cas9, ZFN (zinc finger nuclease), and TALEN (transcription activator like effector nuclease) systems are three potential methods through which CAR-T cells may be generated.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: CD83-Targeted Cell Therapy of Systemic Lupus Erythematosus Using CD4+ T cells, CTLs, and/or T_{reg} Cells Armed with a CD83 CAR

Systemic lupus erythematosus (SLE) is a heterogeneous autoimmune disease capable of catastrophic organ damage. Mortality among SLE patients is over 5 times greater than age-matched controls (Singh RR, et al. Lupus. 2018 27(10):1577-81). Glucocorticoids are often used in treating SLE, but their efficacy is limited, and such broad immunosuppression increases the risk for opportunistic infections (Fanouriakis A, et al. Annals of the Rheumatic Diseases. 2019 78(6):736-45). Long-term exposure to glucocorticoids is also associated with diabetes, osteoporosis, and avascular necrosis. Attempts to test biologic therapeutics in SLE have largely failed, leaving few alternatives to respond to steroid-refractory disease (Fanouriakis A, et al. Annals of the Rheumatic Diseases. 2019 78(6):736-45).

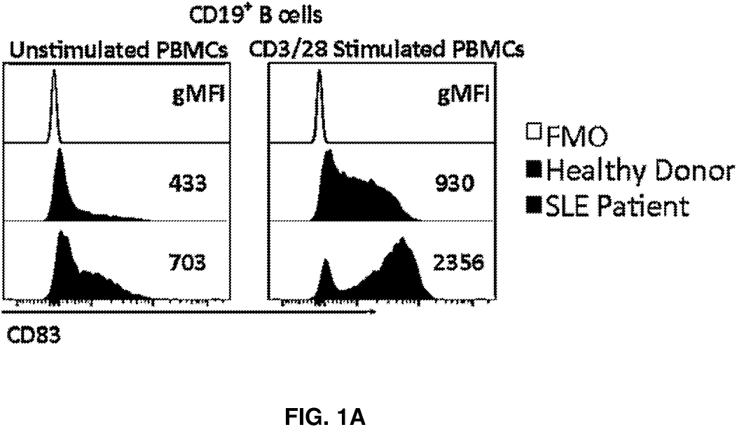

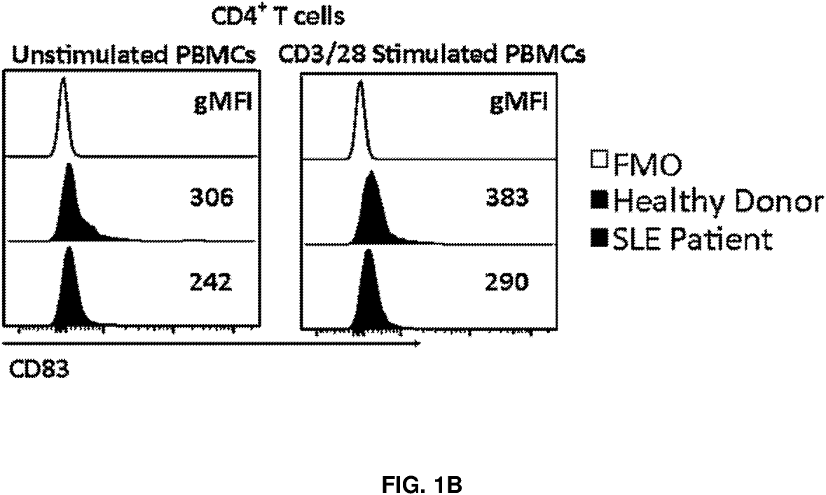

As described herein, CD83 was found to be overexpressed on pathogenic B cells from SLE patients, as compared to healthy donors (FIG. 1A). To treat SLE as described herein, CD83 CAR T cells (e.g., CD4+ T cells, CTLs, and/or T_{reg} cells armed with a CD83 CAR described herein) are generated and administered to an SLE patient. Once administered, the CD83 CAR T cells can reduce the negative effects of CD83+ B cells within the SLE patient, thereby reducing the severity of SLE symptoms within the SLE patient

Moreover, CD83 expression on pathogenic B cells from SLE patients increases when peripheral blood mononuclear cells (PBMC) are stimulated with CD3/CD28 beads (direct stimulation of T cells, and indirect activation of B cells) (FIG. 1A). Unlike GVHD, CD83 is not over expressed on CD4+ conventional T cells in SLE patients (FIG. 1B).

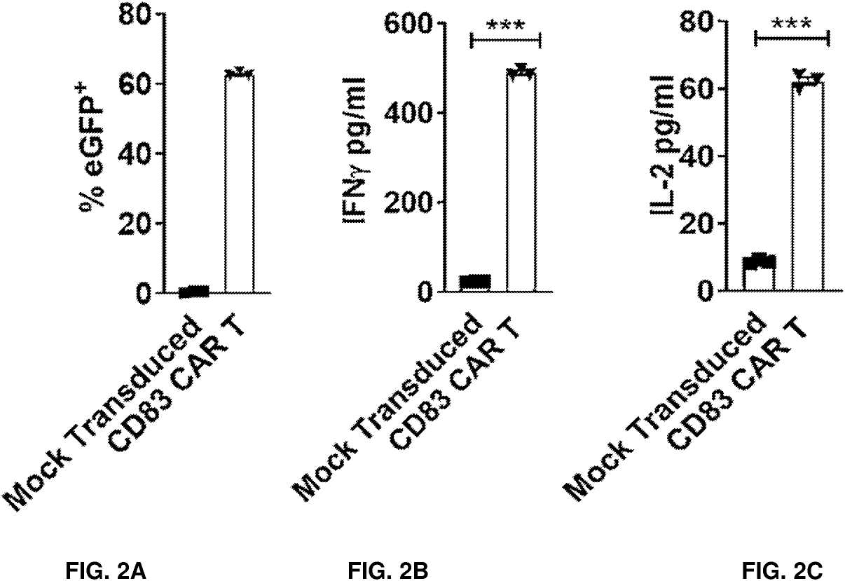

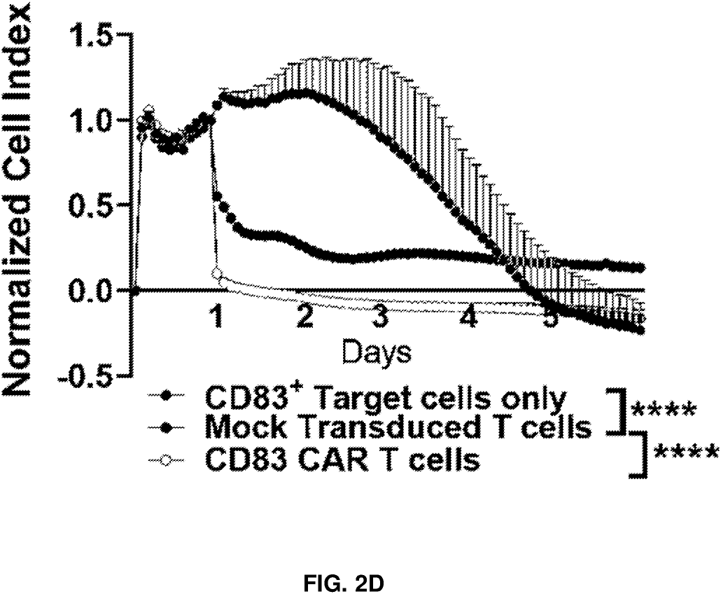

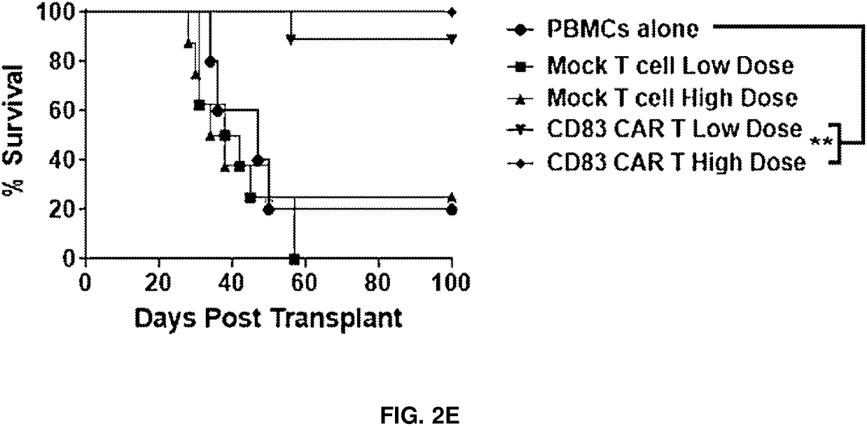

To selectively and efficiently eliminate pathogenic CD83+ effector cells, a human CD83 CAR was developed. The anti-human CD83 single chain variable fragment (scFv) was paired to the CD8 hinge and transmembrane domain, followed by the intracellular 4-1BB co-stimulatory domain and CD3ζ activation domain (Shrestha B, et al. J Clin Invest. 2020 13575). To facilitate tracking of CAR T cells the construct contains an eGFP tag, which can be used to identify the CAR T cell among normal non-CAR T cells. CD83-targeted CAR T cells were retrovirally transduced and generated. The CD83 CAR construct exhibited a high degree of transduction efficiency, with over 60% of T cells expressing eGFP (FIG. 2A). CD83 CAR T cells demonstrated robust IFNγ and IL-2 production when cultured with CD83+ target cells; such as cytokine-matured human, monocyte-derived DCs (moDC) (FIGS. 2B and 2C). CD83 CAR T cells also exhibited potent killing of CD83+ target cells, compared to mock transduced T cells (FIG. 2D). Importantly, CD83 expression on activated CAR T cells is negligible and do not overtly succumb to CD83-mediated fratricide. Further, human CD83 CAR T cells eliminate CD83+ target cells in murine models of xenogeneic GVHD mediated by human T cells and prevent GVHD (FIG. 2E). In part, the CD83 CAR T cells increase the ratio of Tregs to pathogenic conventional T cells.

### Example 2: Bi-specific CAR Cell Therapy of Systemic Lupus Erythematosus

A bispecific CD19/CD83 CAR T cell is tested to selectively eliminate only autoreactive B cells. This approach enhances safety, avoiding off-target effects on CD83+ non-B cell bystander cells or complete B cell aplasia as observed with CD19 CAR T cell alone (FIG. 1A). The objective is to eliminate the need for broad immunosuppression, like steroids, in treating SLE by using CD83-targeted cell therapy.

The disclosed CD19/CD83 CAR T cells only kill targets that co-express CD19 plus CD83, thus avoiding broad B cell aplasia. Moreover, the CD83 construct allows the CAR T cells to eliminate host alloreactive T cells and resists rejection, allowing for the use of off-the-shelf, third-party CD19/CD83 CAR T cells. This is a tremendous translational advantage, as banked, healthy donor CAR T cells can rapidly be used for urgent clinical applications.

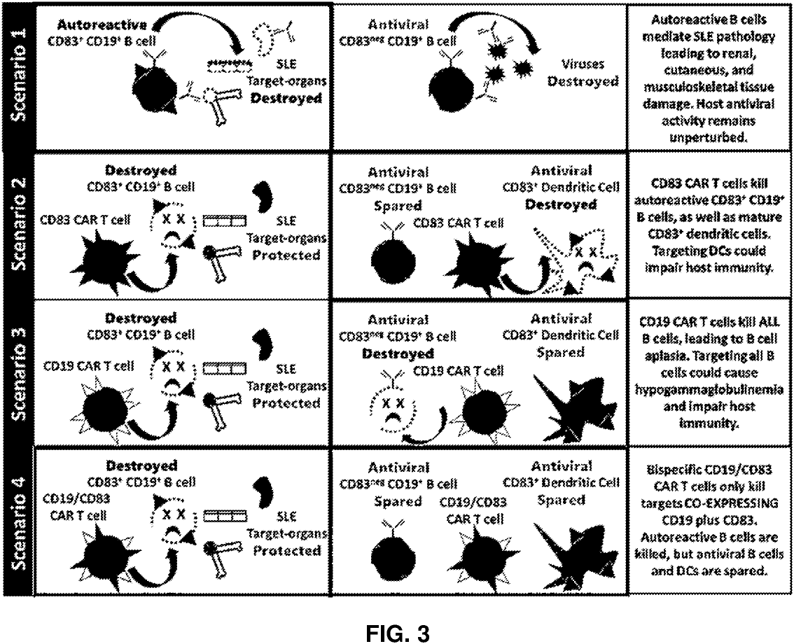

To selectively kill by the CAR T cell, a bispecific CD19/CD83 CAR T cell was developed that requires co-expression of CD19 and CD83 on the target cell, rather than either antigen alone. This approach preserves antiviral immunity by avoiding CD19+ B cell aplasia by CD19 CAR T cells or elimination of mature CD83+ dendritic cells by CD83 CAR T cells, as neither cell type co-express both antigens simultaneously and will be spared by CD19/CD83 CAR T cells (FIG. 3).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A method of inhibiting autoreactive lymphocytes in a subject, the method comprising administering to the subject an effective amount of immune cells genetically modified to express an chimeric antigen receptor (CAR) polypeptide comprising a CD83 antigen binding domain, a transmembrane domain, an intracellular signaling domain, and a co-stimulatory signaling region, thereby inhibiting autoreactive lymphocytes in the subject.

2. The method of claim 1, wherein the subject is a human patient having an autoimmune disease.

3. The method of claim 2, wherein the autoimmune disease is a B cell-mediated autoimmune disease.

4. The method of claim 2, wherein the subject has systemic lupus erythematosus (SLE), and wherein the method treats or prevents one or more symptoms of said SLE in the subject.

5. The method of claim 1, wherein the CAR polypeptide is defined by the formula: 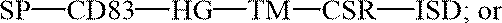 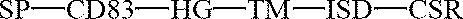
wherein “SP” represents a signal peptide,
wherein “CD83” represents a CD83 antigen binding region,
wherein “HG” represents and optional hinge domain,
wherein “TM” represents a transmembrane domain,
wherein “CSR” represents a co-stimulatory signaling region,
wherein “ISD” represents an intracellular signaling domain, and
wherein 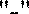
represents an optional bivalent linker.

6. A method of selectively inhibiting autoreactive lymphocytes in a subject, the method comprising administering to the subject an effective amount of immune cells genetically modified to express a first chimeric antigen receptor (CAR) polypeptide comprising a CD83 antigen binding domain and a second CAR polypeptide comprising a CD19 antigen binding domain, thereby selectively inhibiting autoreactive B lymphocytes that express both CD83 and CD19 in the subject.

7. The method of claim 6, wherein:
(a) the first CAR polypeptide is defined by the formula: 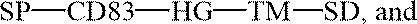
the second CAR polypeptide is defined by the formula: 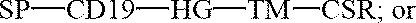
(b) the first CAR polypeptide is defined by the formula: 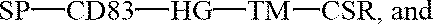
the second CAR polypeptide is defined by the formula: 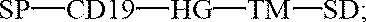
wherein “SP” represents an optional signal peptide,
wherein “CD83” represents a CD83 antigen binding region,
wherein “CD19” represents a CD19 antigen binding region,
wherein “HG” represents an optional hinge domain,
wherein “TM” represents a transmembrane domain,
wherein “CSR” represents one or more co-stimulatory signaling regions,
wherein “SD” represents a signaling domain, and
wherein 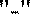
represents an optional peptide bond or linker.

8. The method of claim 1, wherein the intracellular signaling domain comprises a CD3 zeta (CD3ζ) signaling domain.

9. The method of claim 1, wherein the CD83 antigen binding domain is a single-chain variable fragment (scFv) of an antibody that specifically binds CD83.

10. The method of claim 9, wherein the anti-CD83 scFv comprises a variable heavy (V_{H}) domain having heavy chain (HC) CDR1, CDR2 and CDR3 regions; and a variable light (V_{L}) domain having light chain (LC) CDR1, CDR2 and CDR3 regions, wherein:
the HC CDR1 comprises the amino acid sequence SEQ ID NO:1, SEQ ID NO:7, or SEQ ID NO:13;
the HC CDR2 comprises the amino acid sequence SEQ ID NO:2, SEQ ID NO:8, or SEQ ID NO:14;
the HC CDR3 comprises the amino acid sequence SEQ ID NO:3, SEQ ID NO:9, or SEQ ID NO:15;
the LC CDR1 comprises the amino acid sequence SEQ ID NO:4, SEQ ID NO:10, or SEQ ID NO:16;
the LC CDR2 comprises the amino acid sequence SEQ ID NO:5, SEQ ID NO:11, or SEQ ID NO:17; and
the LC CDR3 comprises the amino acid sequence SEQ ID NO:6, SEQ ID NO: 12, or SEQ ID NO:18.

11. The method of claim 10, wherein the anti-CD83 scFv comprises:
a HC CDR1 comprising the amino acid sequence of SEQ ID NO: 1;
a HC CDR2 comprising the amino acid sequence of SEQ ID NO: 2;
a HC CDR2 comprising the amino acid sequence of SEQ ID NO: 3;
a LC CDR1 comprising the amino acid sequence of SEQ ID NO: 4;
a LC CDR2 comprising the amino acid sequence of SEQ ID NO: 5; and
a LC CDR3 comprising the amino acid sequence of SEQ ID NO: 6.

12. The method of claim 10, wherein the anti-CD83 scFv V_{H} domain comprises the amino acid sequence of SEQ ID NO:19, SEQ ID NO:48, SEQ ID NO:49, SEQ ID NO:50, SEQ ID NO:51, SEQ ID NO:52, or SEQ ID NO:53; and/or wherein the anti-CD83 scFv V_{L} domain comprises the amino acid sequence of SEQ ID NO:20, SEQ ID NO:54, or SEQ ID NO:55.

13. The method of claim 12, wherein the anti-CD83 scFv comprises a VH comprising the amino acid sequence of SEQ ID NO:19, and a V_{L} comprising the amino acid sequence of SEQ ID NO:20.

14. The method of claim 9, wherein the anti-CD83 scFv comprises the amino acid sequence of SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, or SEQ ID NO:71; optionally wherein the anti-CD83 scFv comprises the amino acid sequence of SEQ ID NO: 71.

15. The method of claim 1, wherein the costimulatory signaling region is of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof; optionally wherein the costimulatory signaling region is of 4-1BB or CD28.

16. The method of claim 1, wherein the CAR that binds CD83 comprises the amino acid sequence of SEQ ID NO: 90.

17. The method of claim 1, wherein the immune cells comprises T cells, Natural Killer (NK) cells, cytotoxic T lymphocytes (CTLs), and regulatory T cells, or a combination thereof.

18. The method of claim 17, wherein the immune cells comprise cytotoxic T lymphocytes (CTLs).

19. The method of claim 17, wherein the immune cells comprise T cells.

20. A population of immune cells, which are genetically modified to express a first chimeric antigen receptor (CAR) polypeptide comprising a CD83 antigen binding domain and a second CAR polypeptide comprising a CD19 antigen binding domain, wherein the population of immune cells selectively inhibit autoreactive B lymphocytes that express both CD83 and CD19.

21. The population of immune cells of claim 20, wherein:
(a) the first CAR polypeptide is defined by the formula: 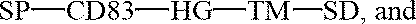
the second CAR polypeptide is defined by the formula: 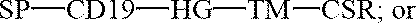
(b) the first CAR polypeptide is defined by the formula: 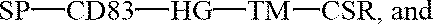
the second CAR polypeptide is defined by the formula: 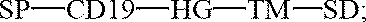
wherein “SP” represents an optional signal peptide,
wherein “CD83” represents a CD83 antigen binding region,
wherein “CD19” represents a CD19 antigen binding region,
wherein “HG” represents an optional hinge domain,
wherein “TM” represents a transmembrane domain,
wherein “CSR” represents one or more co-stimulatory signaling regions, wherein “SD” represents a signaling domain, and
wherein 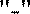
represents an optional peptide bond or linker.

22. The population of immune cells of claim 20, wherein the intracellular signaling domain comprises a CD3 zeta (CD3ζ) signaling domain.

23. The population of immune cells of claim 20, wherein the CD83 antigen binding domain is a single-chain variable fragment (scFv) of an antibody that specifically binds CD83.

24. The population of immune cells of claim 23, wherein the anti-CD83 scFv comprises a variable heavy (V_{H}) domain having heavy chain (HC) CDR1, CDR2 and CDR3 regions; and a variable light (V_{L}) domain having light chain (LC) CDR1, CDR2 and CDR3 regions, wherein:
the HC CDR1 comprises the amino acid sequence SEQ ID NO:1, SEQ ID NO:7, or SEQ ID NO:13;
the HC CDR2 comprises the amino acid sequence SEQ ID NO:2, SEQ ID NO:8, or SEQ ID NO:14;
the HC CDR3 comprises the amino acid sequence SEQ ID NO:3, SEQ ID NO:9, or SEQ ID NO:15;
the LC CDR1 comprises the amino acid sequence SEQ ID NO:4, SEQ ID NO:10, or SEQ ID NO:16;
the LC CDR2 comprises the amino acid sequence SEQ ID NO:5, SEQ ID NO:11, or SEQ ID NO:17; and
the LC CDR3 comprises the amino acid sequence SEQ ID NO:6, SEQ ID NO:12, or SEQ ID NO:18.

25. The population of immune cells of claim 24, wherein the anti-CD83 scFv comprises:
a HC CDR1 comprising the amino acid sequence of SEQ ID NO: 1;
a HC CDR2 comprising the amino acid sequence of SEQ ID NO: 2;
a HC CDR2 comprising the amino acid sequence of SEQ ID NO: 3;
a LC CDR1 comprising the amino acid sequence of SEQ ID NO: 4;
a LC CDR2 comprising the amino acid sequence of SEQ ID NO: 5; and
a LC CDR3 comprising the amino acid sequence of SEQ ID NO: 6.

26. The population of immune cells of claim 25, wherein the anti-CD83 scFv V_{H} domain comprises the amino acid sequence of SEQ ID NO:19, SEQ ID NO:48, SEQ ID NO:49, SEQ ID NO:50, SEQ ID NO:51, SEQ ID NO:52, or SEQ ID NO:53; and/or wherein the anti-CD83 scFv V_{L} domain comprises the amino acid sequence of SEQ ID NO:20, SEQ ID NO:54, or SEQ ID NO:55.

27. The population of immune cells of claim 26, wherein the anti-CD83 scFv comprises a V_{H} comprising the amino acid sequence of SEQ ID NO:19, and a V_{L} comprising the amino acid sequence of SEQ ID NO:20.

28. The population of immune cells of claim 26, wherein the anti-CD83 scFv comprises the amino acid sequence of SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, or SEQ ID NO:71; optionally wherein the anti-CD83 scFv comprises the amino acid sequence of SEQ ID NO: 71.

29. The population of immune cells of claim 20, wherein the costimulatory signaling region is of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof; optionally wherein the costimulatory signaling region is of 4-1BB or CD28.

30. The population of immune cells of claim 20, wherein the CAR that binds CD83 comprises the amino acid sequence of SEQ ID NO: 90.
